(19) United States
(12) Patent Application Publication
(10) Pub. No.: US 2018/0100009 A1
(43) Pub. Date: Apr. 12, 2018

(54) POLYPEPTIDE COMPRISING AN IMMUNOGLOBULIN CHAIN VARIABLE DOMAIN WHICH BINDS TO CLOSTRIDIUM DIFFICILE TOXIN A

(71) Applicant: VHSQUARED LIMITED, Cambridge (GB)

(72) Inventors: Scott Crowe, Cambridge (GB); Mike West, Cambridge (GB); Kevin Roberts, Cambridge (GB); Tim Carlton, Cambridge (GB); Nika Strokappe, Utrecht (NL); Theo Verrips, Utrecht (NL)

(21) Appl. No.: 15/717,221

(22) Filed: Sep. 27, 2017

Related U.S. Application Data
(63) Continuation of application No. PCT/EP2016/057032, filed on Mar. 31, 2016.

(30) Foreign Application Priority Data
Mar. 31, 2015 (EP) 15162116.6

(51) Int. Cl. C07K 16/12; C07K 14/33

(52) U.S. Cl. CPC C07K 16/1282; C07K 14/33; C07K 2317/22; C07K 2317/35; C07K 2317/94; C07K 2317/569; C07K 2317/622; C07K 2317/76; C07K 2317/92; C07K 2317/567

(57) ABSTRACT

There is provided inter alia a polypeptide comprising an immunoglobulin chain variable domain which binds to Clostridium difficile toxin A.

### FIELD OF THE INVENTION

The present invention relates to polypeptides comprising an immunoglobulin chain variable domain (or ‘ICVD’) which binds to Clostridium difficile toxin A (‘TcdA’ or ‘toxin A’) as well as to constructs and pharmaceutical compositions comprising these polypeptides. The present invention also relates to nucleic acids encoding such polypeptides, to methods for preparing such polypeptides, to cDNA and vectors comprising nucleic acids encoding such polypeptides, to host cells expressing or capable of expressing such polypeptides and to uses of such polypeptides, pharmaceutical compositions or constructs.

### BACKGROUND OF THE INVENTION

Clostridium difficile, a spore forming anaerobic bacillus is the causative agent of C. difficile infection. The hospital environment and patients undergoing antibiotic treatment provide a discrete ecosystem where C. difficile persists and selected virulent clones thrive. Consequently, C. difficile is the most frequent cause of nosocomial diarrhoea worldwide. Given the continued use of broad-spectrum antibiotics and the rising numbers of immunocompromised and elderly patients, the problems associated with C. difficile infection are unlikely to recede.

The pathology of C. difficile is mediated by two toxins, toxin A and toxin B and it has been demonstrated that C. difficile ribotypes which lack these toxins do not cause pathogenic infection. Each toxin alone is capable of causing the symptoms of disease. It is believed that the toxins mediate their effect locally by entering the epithelial cells lining the lumen of the colon resulting in cell death, with consequent fluid loss and diarrhoea. There is no consistent pathology associated with toxin entering the systemic circulation and therefore neutralisation of the toxins in the lumen of the colon may provide an effective therapy for this debilitating condition.

Alarmingly, in the last 10 years a new group of highly virulent C. difficile ribotypes has emerged to cause outbreaks of increased disease severity. The most prevalent of the hypervirulent ribotypes is ribotype 027 (strain R20291, Stabler et al 2009 Genome Biology 10:R102) and patients infected with this ribotype exhibit more severe diarrhoea, higher mortality and more recurrences. In Canada, 027 ribotypes were undetected in 2000 but reached 75.2% of all ribotypes isolated by 2003. The 027 ribotype also caused the outbreak of C. difficile at the Stoke Mandeville hospital in the UK. In less than three years a total of 498 patients acquired C. difficile at the hospital of whom 127 died.

The burden of C. difficile disease represented by ribotype 027, other newly emerging hypervirulent ribotypes such as PCR-ribotype 078 (strain ‘M120’, He et al 2010. PNAS. 107 (16) Table S1) and ribotypes such as 087 (strain VPI 10463, Zaib et al 2009 BMC Microbiology 9:6), 017 (strain M68, He et al 2010 PNAS. 107 (16)), 106 (strain Liv22) and 001 (strain Liv24) continue to be a major concern in hospitals throughout the developed world.

Current therapy for C. difficile involves a course of either vancomycin or metronidazole, which in the majority of patients resolves the symptoms and diarrhoea. Unfortunately on cessation of these antibiotics, 20-30% of patients relapse and a vicious cycle of further antibiotic courses and relapses can follow. Recurrence rates are worse with patients infected with the hypervirulent 027 ribotype.

Whilst there are new treatments in development for C. difficile, effectiveness against multiple ribotypes remains a challenge. In 2011 a new antibiotic, Fidaxomicin, the first new treatment for C. difficile in more than 20 years, was approved by the FDA and EMEA. Although Fidaxomicin demonstrated a reduced rate of recurrence in non-027 infected patients, it did not show any superiority versus vancomycin for patients infected with the 027 hypervirulent ribotype.

Merck have completed a Phase 2 trial of a combination of anti toxin A and anti toxin B antibodies administered systemically. These antibodies were originally developed by Medarex. Comparative experiments provided herein demonstrate that constructs of the present invention have a higher potency than Medarex-developed antibody 3D8 against toxins of multiple ribotypes including 027.

There is, therefore, an overwhelming unmet need for therapies that will reduce relapse rates in patients infected with C. difficile and especially those infected with the hypervirulent 027 ribotype. Safe and effective therapeutics must be developed which are capable of neutralising TcdA from a broad range of C. difficile ribotypes, including TcdA from ribotype 027. This is particularly challenging due to the significant sequence variation of toxins produced by different ribotypes of C. difficile (see Table 1C below).

WO 2006/121422 discloses antibodies that specifically bind to toxins of C. difficile, antigen binding portions thereof, and methods of making and using said antibodies and antigen binding portions. WO 2011/130650 discloses regents, compositions and therapies with which to treat C. difficile infection and related disease conditions and pathologies, including in particular antibodies or antigen-binding fragments thereof that bind specifically to TcdA and/or TcdB of C. difficile and neutralise the activities of these toxins. WO 2012/055030 discloses C. difficile toxin-specific antibodies, compositions and uses thereof, which toxin-specific antibodies may be specific for either TcdA or TcdB.

Polypeptides of the present invention may, in at least some embodiments, have one or more of the following advantages compared to anti-TcdA substances of the prior art:
(i) increased affinity for TcdA;
(ii) increased specificity for TcdA;
(iii) increased neutralising capability against TcdA;
(iv) increased cross-reactivity with TcdA from multiple different ribotypes of C. difficile, particularly ribotypes 087, 027, 078, 106 and 001;
(v) reduced immunogenicity when administered to a human;
(vi) increased stability in the presence of proteases, for example (a) in the presence of proteases present in the small and/or large intestine and/or C. difficile-specific proteases and/or inflammatory proteases, for example enteropeptidase, trypsin and chymotrypsin and/or (b) in the presence of proteases from intestinal commensal microflora and/or pathogenic bacteria, actively secreted and/or released by lysis of microbial cells found in the small and/or large intestine;
(vii) increased stability to protease degradation during production (for example resistance to yeast proteases);
(viii) increased suitability for oral administration;
(ix) increased suitability for local delivery to the intestinal tract following oral administration;
(x) increased suitability for expression, in a heterologous host such as bacteria such as Escherichia coli and/or a yeast such as a yeast belonging to the genera Aspergillus, Saccharomyces, Kluyveromyces, Hansenula or Pichia, such as Saccharomyces cerevisiae or Pichia pastoris;
(xi) suitability for, and improved properties for, use in a pharmaceutical;
(xii) suitability for, and improved properties for, use in a functional food;
(xiii) increased suitability for formatting in a multispecific format;
(xiv) binding to novel, advantageous epitopes.

Advantages (i) to (xiv) above may potentially be realised by the polypeptides of the present invention in a monovalent format or in a multivalent format such as a bihead format (for example homobihead or heterobihead formats) or a quadrahead format.

### SUMMARY OF THE INVENTION

The present inventors have produced surprisingly advantageous polypeptides comprising immunoglobulin chain variable domains which bind to TcdA. These polypeptides, in preferred embodiments, benefit from surprisingly high potency against TcdA from multiple ribotypes of C. difficile and more particularly remain stable on exposure to proteases such as trypsin, chymotrypsin and/or proteases present in the small and large intestine. In one embodiment, these polypeptides have undergone further enhancement by engineering. It is expected that these further enhanced polypeptides benefit from the above advantages, retain their TcdA-neutralising activity during passage through the intestinal tract and further resist degradation and/or inactivation by proteases present in the intestinal tract. It may be expected that these polypeptides have particular utility in the prevention or treatment of C. difficile infection, particularly when administered orally.

The present invention provides a polypeptide comprising an immunoglobulin chain variable domain which binds to Clostridium difficile toxin A, wherein the immunoglobulin chain variable domain comprises three complementarity determining regions (CDR1-CDR3) and four framework regions (FR1-FR4), wherein:

(a) CDR1 comprises a sequence sharing 40% or greater sequence identity with SEQ ID NO: 1, CDR2 comprises a sequence sharing 55% or greater sequence identity with SEQ ID NO: 2 and CDR3 comprises a sequence sharing 55% or greater sequence identity with SEQ ID NO: 3; or

(b) CDR1 comprises a sequence sharing 40% or greater sequence identity with SEQ ID NO: 4, CDR2 comprises a sequence sharing 55% or greater sequence identity with SEQ ID NO: 5 and CDR3 consists of a sequence having 2 to 4 residues and sharing 25% or greater sequence identity with the sequence EYY; or

(c) CDR1 comprises a sequence sharing 40% or greater sequence identity with SEQ ID NO: 6, CDR2 comprises a sequence sharing 55% or greater sequence identity with SEQ ID NO: 7 and CDR3 comprises a sequence sharing 50% or greater sequence identity with SEQ ID NO: 8; or

(d) CDR1 comprises a sequence sharing 40% or greater sequence identity with SEQ ID NO: 9, CDR2 comprises a sequence sharing 55% or greater sequence identity with SEQ ID NO: 10 and CDR3 consists of a sequence having 2 to 4 residues and sharing 25% or greater sequence identity with the sequence MWY; or

(e) CDR1 comprises a sequence sharing 40% or greater sequence identity with SEQ ID NO: 11, CDR2 comprises a sequence sharing 55% or greater sequence identity with SEQ ID NO: 12 and CDR3 comprises a sequence sharing 60% or greater sequence identity with SEQ ID NO: 13.

In order to facilitate understanding of the invention and with no limiting effect, options (a)-(e) are related to the following ICVD sequences:

| | Related |
|---|---|
| ICVD label | embodiment |
| B4F10arm | a |
| Q34A3arm | b |
| B14G12arm | c |
| B14G3arm | d |
| Q30A5arm | e |

### DESCRIPTION OF THE FIGURES

FIG. 1 % neutralisation of multiple TcdA ribotypes by B4F10 family members at single concentrations

FIG. 2 Example TcdA dose-response curve on Vero cells

FIGS. 3A-E Dose response curves of multiple TcdA ribotypes by unmodified ICVDs

FIGS. 4A-4B Dose response curves of multiple TcdA ribotypes by ID12A and ID17A

FIGS. 5A-5E Dose response curves of multiple TcdA ribotypes by Q34A3, ID1A, ID2A and ID3A

FIGS. 6A-6B Dose response curves of multiple TcdA ribotypes by ID10A, ID11A, ID13A and ID15A

FIG. 7 Dose response curves of multiple TcdA ribotypes by ID1A compared to ID12A

FIGS. 8A-8B Dose response curves of multiple TcdA ribotypes by ID1A compared to mAb 3D8

FIGS. 9A-9J Dose response curves of multiple TcdA ribotypes by ID29A-ID34A

FIG. 10 Dose response curves of multiple TcdA ribotypes by ID33A compared to mAb 3D8

FIG. 11 Results of incubation of ID17A and ID33A with trypsin and chymotrypsin beads

FIG. 12 Results of incubation of ID17A and ID33A in faecal supernatant pools

FIGS. 13A-13B Dose response curves of multiple TcdA and TcdB ribotypes by ID1C and ID3C

FIGS. 14A-14B Dose response curves of ribotype 027 TcdA and TcdB by ID5C

FIGS. 15A-15C Dose response curves of multiple TcdA ribotypes by ID8C compared to ID33A

FIGS. 16A-16E Dose response curves of multiple TcdB ribotypes by ID8C compared to ID43A

FIGS. 17A-17B Dose response curves of ribotype 027 TcdA and TcdB by ID6C

FIG. 18 Dose response curves of multiple TcdA ribotypes by ID7C compared to ID17A

FIGS. 19A-19C Dose response curves of multiple TcdB ribotypes by ID7C compared to ID41A

FIGS. 20A-20B Dose response curves of ribotype 027 and 087 TcdA and TcdB by ID11C

FIG. 21 Dose response curves for simultaneous binding of 027 TcdA and TcdB by ID1C

FIG. 22 Dose response curves for simultaneous binding of 087 TcdA and TcdB by ID1C

FIG. 23 Dose response curves for simultaneous binding of 027 TcdA and TcdB by ID3C

FIG. 24 Dose response curves for simultaneous binding of 087 TcdA and TcdB by ID3C

### DESCRIPTION OF THE SEQUENCES

SEQ ID NO: 1—Polypeptide sequence of CDR1 B4F10arm

SEQ ID NO: 2—Polypeptide sequence of CDR2 B4F10arm

SEQ ID NO: 3—Polypeptide sequence of CDR3 B4F10arm

SEQ ID NO: 4—Polypeptide sequence of CDR1 Q34A3arm

SEQ ID NO: 5—Polypeptide sequence of CDR2 Q34A3arm

SEQ ID NO: 6—Polypeptide sequence of CDR1 B14G12arm

SEQ ID NO: 7—Polypeptide sequence of CDR2 B14G12arm

SEQ ID NO: 8—Polypeptide sequence of CDR3 B14G12arm

SEQ ID NO: 9—Polypeptide sequence of CDR1 B14G3arm

SEQ ID NO: 10—Polypeptide sequence of CDR2 B14G3arm

SEQ ID NO: 11—Polypeptide sequence of CDR1 Q30A5

SEQ ID NO: 12—Polypeptide sequence of CDR2 Q30A5arm

SEQ ID NO: 13—Polypeptide sequence of CDR3 Q30A5arm

SEQ ID NO: 14—Polypeptide sequence of B4F10arm

SEQ ID NO: 15—Polypeptide sequence of Q34A3arm

SEQ ID NO: 16—Polypeptide sequence of B14G12arm

SEQ ID NO: 17—Polypeptide sequence of B14G3arm

SEQ ID NO: 18—Polypeptide sequence of Q30A5arm

SEQ ID NO: 19—Polypeptide sequence of B4F10

SEQ ID NO: 20—Polypeptide sequence of B1A7

SEQ ID NO: 21—Polypeptide sequence of B2H11

SEQ ID NO: 22—Polypeptide sequence of B3D5

SEQ ID NO: 23—Polypeptide sequence of B3H7

SEQ ID NO: 24—Polypeptide sequence of B3H11

SEQ ID NO: 25—Polypeptide sequence of B4F5

SEQ ID NO: 26—Polypeptide sequence of B4F7

SEQ ID NO: 27—Polypeptide sequence of B4G7

SEQ ID NO: 28—Polypeptide sequence of B4H7

SEQ ID NO: 29—Polypeptide sequence of Q34A3

SEQ ID NO: 30—Polypeptide sequence of B14G12

SEQ ID NO: 31—Polypeptide sequence of B14G3

SEQ ID NO: 32—Polypeptide sequence of Q30A5

SEQ ID NO: 33—Polypeptide sequence of ID1A

SEQ ID NO: 34—Polypeptide sequence of ID2A

SEQ ID NO: 35—Polypeptide sequence of ID3A

SEQ ID NO: 36—Polypeptide sequence of ID10A

SEQ ID NO: 37—Polypeptide sequence of ID11A

SEQ ID NO: 38—Polypeptide sequence of ID12A

SEQ ID NO: 39—Polypeptide sequence of ID13A

SEQ ID NO: 40—Polypeptide sequence of ID15A

SEQ ID NO: 41—Polypeptide sequence of ID17A

SEQ ID NO: 42—Polypeptide sequence of ID29A

SEQ ID NO: 43—Polypeptide sequence of ID30A

SEQ ID NO: 44—Polypeptide sequence of ID31A

SEQ ID NO: 45—Polypeptide sequence of ID32A

SEQ ID NO: 46—Polypeptide sequence of ID33A

SEQ ID NO: 47—Polypeptide sequence of ID34A

SEQ ID NO: 48—Polypeptide sequence of ID35A

SEQ ID NO: 49—Polynucleotide sequence of 3′ primer mentioned under Preparative Methods section

SEQ ID NO: 50—Polynucleotide sequence of M13.rev used in Example 2

SEQ ID NO: 51—Polynucleotide sequence of M13.fw used in Example 2

SEQ ID NO: 52—Polynucleotide sequence encoding ID1A

SEQ ID NO: 53—Polynucleotide sequence encoding ID2A

SEQ ID NO: 54—Polynucleotide sequence encoding ID10A

SEQ ID NO: 55—Polynucleotide sequence encoding ID11A

SEQ ID NO: 56—Polynucleotide sequence encoding ID12A

SEQ ID NO: 57—Polynucleotide sequence encoding ID13A

SEQ ID NO: 58—Polynucleotide sequence encoding ID15A

SEQ ID NO: 59—Polynucleotide sequence encoding ID17A

SEQ ID NO: 60—Polynucleotide sequence encoding ID33A

SEQ ID NO: 61—Polynucleotide sequence encoding Q34A3arm (in ID33A)

SEQ ID NO: 62—Polynucleotide sequence encoding B4F10arm (in ID33A)

SEQ ID NO: 63—Polypeptide sequence of ID1C

SEQ ID NO: 64—Polypeptide sequence of ID3C

SEQ ID NO: 65—Polypeptide sequence of ID5C

SEQ ID NO: 66—Polypeptide sequence of ID6C

SEQ ID NO: 67—Polypeptide sequence of ID7C

SEQ ID NO: 68—Polypeptide sequence of ID8C

SEQ ID NO: 69—Polypeptide sequence of ID11C

SEQ ID NO: 70—Polypeptide sequence of Q31 B1 (anti-TcdB ICVD)

SEQ ID NO: 71—Polypeptide sequence of B10F1 (anti-TcdB ICVD)

SEQ ID NO: 72—Polypeptide sequence of ID43B (anti-TcdB bihead)

SEQ ID NO: 73—Polypeptide sequence of ID41B (anti-TcdB bihead)

SEQ ID NO: 74—Polypeptide sequence of TcdA from C. difficile ribotype 087

SEQ ID NO: 75—Polypeptide sequence of TcdA from C. difficile ribotype 078

SEQ ID NO: 76—Polypeptide sequence of TcdA from C. difficile ribotype 027

### DETAILED DESCRIPTION OF THE INVENTION

### Polypeptides Including Antibodies and Antibody Fragments Including ICVDs such as the VH and VHH

A conventional antibody or immunoglobulin (Ig) is a protein comprising four polypeptide chains: two heavy (H) chains and two light (L) chains. Each chain is divided into a constant region and a variable domain. The heavy chain variable domains are abbreviated herein as VHC, and the light (L) chain variable domains are abbreviated herein as VLC. These domains, domains related thereto and domains derived therefrom, are referred to herein as immunoglobulin chain variable domains. The VHC and VLC domains can be further subdivided into regions of hypervariability, termed “complementarity determining regions” (“CDRs”), interspersed with regions that are more conserved, termed “framework regions” (“FRs”). The framework and complementarity determining regions have been precisely defined (Kabat et al 1991 Sequences of Proteins of Immunological Interest, Fifth Edition U.S. Department of Health and Human Services, NIH Publication Number 91-3242, herein incorporated by reference in its entirety). In a conventional antibody, each VHC and VLC is composed of three CDRs and four FRs, arranged from amino-terminus to carboxy-terminus in the following order: FR1, CDR1, FR2, CDR2, FR3, CDR3, FR4. The conventional antibody tetramer of two heavy immunoglobulin chains and two light immunoglobulin chains is formed with the heavy and the light immunoglobulin chains inter-connected by e.g. disulfide bonds, and the heavy chains similarity connected. The heavy chain constant region includes three domains, CH1, CH2 and CH3. The light chain constant region is comprised of one domain, CL. The variable domain of the heavy chains and the variable domain of the light chains are binding domains that interact with an antigen. The constant regions of the antibodies typically mediate the binding of the antibody to host tissues or factors, including various cells of the immune system (e.g. effector cells) and the first component (C1q) of the classical complement system. The term antibody includes immunoglobulins of types IgA, IgG, IgE, IgD, IgM (as well as subtypes thereof), wherein the light chains of the immunoglobulin may be kappa or lambda types. The overall structure of immunoglobulin-gamma (IgG) antibodies assembled from two identical heavy (H)-chain and two identical light (L)-chain polypeptides is well established and highly conserved in mammals (Padlan 1994 Mol Immunol 31:169-217).

An exception to conventional antibody structure is found in sera of Camelidae. In addition to conventional antibodies, these sera possess special IgG antibodies. These IgG antibodies, known as heavy-chain antibodies (HCAbs), are devoid of the L chain polypeptide and lack the first constant domain (CH1). At its N-terminal region, the H chain of the homodimeric protein contains an immunoglobulin chain variable domain, referred to as the VHH, which serves to associate with its cognate antigen (Muyldermans 2013 Annu Rev Biochem 82:775-797, Hamers-Casterman et al 1993 Nature 363(6428):446-448, Muyldermans et al 1994 Protein Eng 7(9):1129-1135, herein incorporated by reference in their entirety).

An antigen-binding fragment (or “antibody fragment” or “immunoglobulin fragment”) as used herein refers to a portion of an antibody that specifically binds to TcdA (e.g. a molecule in which one or more immunoglobulin chains is not full length, but which specifically binds to TcdA). Examples of binding fragments encompassed within the term antigen-binding fragment include:

(i) a Fab fragment (a monovalent fragment consisting of the VLC, VHC, CL and CH1 domains);

(ii) a F(ab′)2 fragment (a bivalent fragment comprising two Fab fragments linked by a disulfide bridge at the hinge region);

(iii) a Fd fragment (consisting of the VHC and CH1 domains);

(iv) a Fv fragment (consisting of the VLC and VHC domains of a single arm of an antibody);

(v) an scFv fragment (consisting of VLC and VHC domains joined, using recombinant methods, by a synthetic linker that enables them to be made as a single protein chain in which the VLC and VHC regions pair to form monovalent molecules);

(vi) a VH (an immunoglobulin chain variable domain consisting of a VHC domain (Ward et al Nature 1989 341:544-546);

(vii) a VL (an immunoglobulin chain variable domain consisting of a VLC domain);

(viii) a V-NAR (an immunoglobulin chain variable domain consisting of a VHC domain from chondrichthyes IgNAR (Roux et al 1998 Proc Natl Acad Sci USA 95:11804-11809 and Griffiths et al 2013 Antibodies 2:66-81, herein incorporated by reference in their entirety)

(ix) a VHH.

The total number of amino acid residues in an immunoglobulin chain variable domain such as a VHH or VH may be in the region of 110-130.

Immunoglobulin chain variable domains of the invention may for example be obtained by preparing a nucleic acid encoding an immunoglobulin chain variable domain using techniques for nucleic acid synthesis, followed by expression of the nucleic acid thus obtained According to a specific embodiment, an immunoglobulin chain variable domain of the invention does not have an amino acid sequence which is exactly the same as (i.e. shares 100% sequence identity with) the amino acid sequence of a naturally occurring polypeptide such as a VH or VHH domain of a naturally occurring antibody.

Substituting at least one amino acid residue in the framework region of a non human immunoglobulin variable domain with the corresponding residue from a human variable domain is humanisation. Humanisation of a variable domain may reduce immunogenicity in humans.

Suitably the polypeptide of the present invention consists of an immunoglobulin chain variable domain. Suitably, the polypeptide of the present invention is an antibody or an antibody fragment. Suitably the antibody fragment is a VHH, a VH, a VL, a V-NAR, a Fab fragment, a VL or a F(ab′)2 fragment (such as a VHH or VH, most suitably a VHH).

### Specificity, Affinity, Avidity and Cross-Reactivity

Specificity refers to the number of different types of antigens or antigenic determinants to which a particular antigen-binding polypeptide can bind. The specificity of an antigen-binding polypeptide is the ability of the antigen-binding polypeptide to recognise a particular antigen as a unique molecular entity and distinguish it from another.

Affinity, represented by the equilibrium constant for the dissociation of an antigen with an antigen-binding polypeptide (Kd), is a measure of the binding strength between an antigenic determinant and an antigen-binding site on the antigen-binding polypeptide: the lesser the value of the Kd, the stronger the binding strength between an antigenic determinant and the antigen-binding polypeptide (alternatively, the affinity can also be expressed as the affinity constant (Ka), which is 1/Kd). Affinity can be determined by known methods, depending on the specific antigen of interest.

Avidity is the measure of the strength of binding between an antigen-binding polypeptide and the pertinent antigen. Avidity is related to both the affinity between an antigenic determinant and its antigen binding site on the antigen-binding polypeptide and the number of pertinent binding sites present on the antigen-binding polypeptide.

Suitably, antigen-binding polypeptides of the invention will bind with a dissociation constant (Kd) of 10⁻⁶ to 10⁻¹² M, more suitably 10⁻⁷ to 10⁻¹² M, more suitably 10⁻⁸ to 10⁻¹² M and more suitably 10⁻⁹ to 10⁻¹² M.

Any Kd value less than 10⁻⁶ is considered to indicate binding. Specific binding of an antigen-binding polypeptide to an antigen or antigenic determinant can be determined in any suitable known manner, including, for example, Scatchard analysis and/or competitive binding assays, such as radioimmunoassays (RIA), enzyme immunoassays (EIA) and sandwich competition assays, and the different variants thereof known in the art.

An anti-TcdA polypeptide, a polypeptide which interacts with TcdA, or a polypeptide against TcdA, are all effectively polypeptides which bind to TcdA. A polypeptide of the invention may bind to a linear or conformational epitope on TcdA. The term “binds to TcdA” means binding to any one or more of the N-terminal, hydrophobic or C-terminal domains of TcdA.

Suitably, the polypeptide of the invention is capable of neutralising TcdA from multiple ribotypes of C. difficile. More suitably, the polypeptide of the invention is capable of neutralising TcdA from ribotypes 087, 027, 078, 017, 106 and 001. More suitably, the polypeptide of the invention will neutralise TcdA from ribotype 027.

Suitably the polypeptide of the invention is directed against one or more epitopes on TcdA such that said polypeptide of the invention, upon binding to TcdA, is capable of inhibiting or reducing the cytotoxic effect that is mediated by said TcdA. Suitably, the polypeptide of the invention binds to the cell binding domain of Clostridium difficile toxin A.

The polypeptides of the present invention bind to one or more epitope(s) on TcdA. In one aspect of the invention there is provided a polypeptide which binds to the same epitope on TcdA as B4F10arm, B4F10, B1A7, B2H11, B3D5, B3H7, B3H11, B4F5, B4F7, B4G7, B4H7, Q34A3arm, Q34A3, ID1A, ID2A, ID10A, B14G12arm, B14G12, B14G3arm, B14G3, Q30A5arm, Q30A5, ID3A, ID11A, ID12A, ID13A, ID15A, ID17A, ID29A, ID30A, ID31A, ID32A, ID33A or ID34A.

Suitably, the polypeptide of the invention is isolated. An “isolated” polypeptide is one that is removed from its original environment. For example, a naturally-occurring polypeptide of the invention is isolated if it is separated from some or all of the coexisting materials in the natural system.

### Potency, Inhibition and Neutralisation

Potency is a measure of the activity of a therapeutic agent expressed in terms of the amount required to produce an effect of given intensity. A highly potent agent evokes a greater response at low concentrations compared to an agent of lower potency that evokes a smaller response at low concentrations. Potency is a function of affinity and efficacy. Efficacy refers to the ability of therapeutic agent to produce a biological response upon binding to a target ligand and the quantitative magnitude of this response. The term half maximal effective concentration (EC50) refers to the concentration of a therapeutic agent which causes a response halfway between the baseline and maximum after a specified exposure time. The therapeutic agent may cause inhibition or stimulation. It is commonly used, and is used herein, as a measure of potency.

A neutralising polypeptide for the purposes of the invention is a polypeptide which defends a cell from the effects of TcdA by, for example, inhibiting the biological effect of TcdA. The effectiveness of an anti-TcdA therapeutic agent can be ascertained using a neutralisation assay. A particularly suitable neutralisation assay is the measurement of Vero cell viability using Alamar Blue (Fields and Lancaster American Biotechnology Laboratory 1993 11(4):48-50). Using a range of known concentrations of anti-TcdA polypeptide, this assay can be performed to ascertain the ability of an anti-TcdA polypeptide to neutralise the effects of TcdA cytotoxicity by producing a dose-response curve and/or by ascertaining the half maximal effective concentration (EC50) of the anti-TcdA polypeptide. This standard Vero cell assay is used herein and detailed further in the Examples section below.

Suitably the polypeptide or construct of the invention neutralises TcdA cytotoxicity (such as TcdA ribotype 087, 027, 078, 017, 106 and 001) in the standard Vero cell assay with an EC50 of 50000 pM or less, such as 40000 pM or less, such as 30000 pM or less, such as 20000 pM or less, such as 10000 pM or less, such as 5000 pM or less, such as 4000 pM or less, such as 3000 pM or less, such as 2000 pM or less, such as 1000 pM or less, such as 500 pM or less, such as 250 pM or less, such as 100 pM or less, such as 80 pM or less, such as 60 pM or less, such as 40 pM or less, such as 30 pM or less, such as 20 pM or less, such as 10 pM or less.

### Polypeptide and Polynucleotide Sequences

For the purposes of comparing two closely-related polypeptide sequences, the “% sequence identity” between a first polypeptide sequence and a second polypeptide sequence may be calculated using NCBI BLAST v2.0, using standard settings for polypeptide sequences (BLASTP). For the purposes of comparing two closely-related polynucleotide sequences, the “% sequence identity” between a first nucleotide sequence and a second nucleotide sequence may be calculated using NCBI BLAST v2.0, using standard settings for nucleotide sequences (BLASTN).

Polypeptide or polynucleotide sequences are said to be the same as or identical to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides.

A “difference” between sequences refers to an insertion, deletion or substitution of a single amino acid residue in a position of the second sequence, compared to the first sequence. Two polypeptide sequences can contain one, two or more such amino acid differences. Insertions, deletions or substitutions in a second sequence which is otherwise identical (100% sequence identity) to a first sequence result in reduced % sequence identity. For example, if the identical sequences are 9 amino acid residues long, one substitution in the second sequence results in a sequence identity of 88.9%. If the identical sequences are 17 amino acid residues long, two substitutions in the second sequence results in a sequence identity of 88.2%. If the identical sequences are 7 amino acid residues long, three substitutions in the second sequence results in a sequence identity of 57.1%. If first and second polypeptide sequences are 9 amino acid residues long and share 6 identical residues, the first and second polypeptide sequences share greater than 66% identity (the first and second polypeptide sequences share 66.7% identity). If first and second polypeptide sequences are 17 amino acid residues long and share 16 identical residues, the first and second polypeptide sequences share greater than 94% identity (the first and second polypeptide sequences share 94.1% identity). If first and second polypeptide sequences are 7 amino acid residues long and share 3 identical residues, the first and second polypeptide sequences share greater than 42% identity (the first and second polypeptide sequences share 42.9% identity).

Alternatively, for the purposes of comparing a first, reference polypeptide sequence to a second, comparison polypeptide sequence, the number of additions, substitutions and/or deletions made to the first sequence to produce the second sequence may be ascertained. An addition is the addition of one amino acid residue into the sequence of the first polypeptide (including addition at either terminus of the first polypeptide). A substitution is the substitution of one amino acid residue in the sequence of the first polypeptide with one different amino acid residue. A deletion is the deletion of one amino acid residue from the sequence of the first polypeptide (including deletion at either terminus of the first polypeptide).

For the purposes of comparing a first, reference polynucleotide sequence to a second, comparison polynucleotide sequence, the number of additions, substitutions and/or deletions made to the first sequence to produce the second sequence may be ascertained. An addition is the addition of one nucleotide residue into the sequence of the first polynucleotide (including addition at either terminus of the first polynucleotide). A substitution is the substitution of one nucleotide residue in the sequence of the first polynucleotide with one different nucleotide residue. A deletion is the deletion of one nucleotide residue from the sequence of the first polynucleotide (including deletion at either terminus of the first polynucleotide).

A “conservative” amino acid substitution is an amino acid substitution in which an amino acid residue is replaced with another amino acid residue of similar chemical structure and which is expected to have little influence on the function, activity or other biological properties of the polypeptide. Such conservative substitutions suitably are substitutions in which one amino acid within the following groups is substituted by another amino acid residue from within the same group:

| Group | Amino acid residue |
|---|---|
| Non-polar aliphatic | Glycine |
| | Alanine |
| | Valine |
| | Leucine |
| | Isoleucine |
| Aromatic | Phenylalanine |
| | Tyrosine |
| | Tryptophan |
| Polar uncharged | Serine |
| | Threonine |
| | Asparagine |
| | Glutamine |
| Negatively charged | Aspartate |
| | Glutamate |
| Positively charged | Lysine |
| | Arginine |

As used herein, numbering of polypeptide sequences and definitions of CDRs and FRs are as defined according to the Kabat system (Kabat et al 1991 Sequences of Proteins of

Immunological Interest, Fifth Edition U.S. Department of Health and Human Services, NIH Publication Number 91-3242, in conjunction with the methods for analysis of antibody sequence and structure described in Martin 2010 ‘Protein sequence and structure of antibody variable domains’, Antibody Engineering volume 2, both herein incorporated by reference in their entirety). A “corresponding” amino acid residue between a first and second polypeptide sequence is an amino acid residue in a first sequence which shares the same position according to the Kabat system with an amino acid residue in a second sequence, whilst the amino acid residue in the second sequence may differ in identity from the first. Suitably corresponding residues will share the same number (and letter) if the framework and CDRs are the same length according to Kabat definition. Alignment can be achieved manually or by using, for example, a known computer algorithm for sequence alignment such as NCBI BLAST v2.0 (BLASTP or BLASTN) using standard settings.

Suitably, the polynucleotides used in the present invention are isolated. An “isolated” polynucleotide is one that is removed from its original environment. For example, a naturally-occurring polynucleotide is isolated if it is separated from some or all of the coexisting materials in the natural system. A polynucleotide is considered to be isolated if, for example, it is cloned into a vector that is not a part of its natural environment or if it is comprised within cDNA.

In one aspect of the invention there is provided a polynucleotide encoding the polypeptide or construct of the invention. Suitably the polynucleotide comprises or consists of a sequence sharing 70% or greater, such as 80% or greater, such as 90% or greater, such as 95% or greater, such as 99% or greater sequence identity with any one of SEQ ID NOs: 52-62. More suitably the polynucleotide comprises or consists of any one of SEQ ID NOs: 52-62. In a further aspect there is provided a cDNA comprising said polynucleotide.

In one aspect of the invention there is provided a polynucleotide comprising or consisting of a sequence sharing 70% or greater, such as 80% or greater, such as 90% or greater, such as 95% or greater, such as 99% or greater sequence identity with any one of the portions of any one of SEQ ID NOs: 52-62 which encodes CDR1, CDR2 or CDR3 of the encoded immunoglobulin chain variable domain.

Suitably, the polypeptide sequence of the present invention contains at least one alteration with respect to a native sequence. Suitably, the polynucleotide sequences of the present invention contain at least one alteration with respect to a native sequence. Suitably the alteration to the polypeptide sequence or polynucleotide sequence is made to increase stability of the polypeptide or encoded polypeptide to proteases present in the intestinal tract.

TABLE 1A
Kabat characterisation system applied to ICVD and ICVD construct sequences

CDRs 1, 2 and 3 are the first, second and third underlined portions of each ICVD. FRs 1, 2, 3
and 4 are the first, second, third and fourth portions joining the CDRs of each ICVD. The
linker is also shown in the case of homobiheads or heterobiheads. Bold residues are substitutions
of wild type residues. Substitution descriptions in brackets are referred-to by N-to-C-terminal
numbering (as opposed to Kabat numbering).
ID33A (B4F10arm + Q34A3arm with [G₄S]₄ linker) (SEQ ID NO: 46)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SHSDCIGYGCHHVSQDY WGQGTQVTVSS GGGGSGGGGSGGGGSGGGGS
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
B4F10arm (SEQ ID NO: 14)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SHSDCIGYGCHHVSQDY WGQGTQVTVSS
Q34A3arm (SEQ ID NO: 15)
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
B4F10 (unmodified) (SEQ ID NO: 19)
QVQLQESGGGLVQAGGSLRLSCAASGRTSD VYAMG WFRQVPGKEREFVA TINRSGSDSYYADSVKG
RFTISRDNAKNTVYLQMNSLKPEETAVYYCAA SRSDCIGYGCRRVSQDY WGQGTQVTVSS
Q34A3 (unmodified) (SEQ ID NO: 29)
EVQLVESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
ID1A (Q34A3 with E1D and V5Q) (SEQ ID NO: 33)
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
ID2A (ID1A with P14A) (SEQ ID NO: 34)
DVQLQESGGGLVQAGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
ID3A (homobihead of ID1A with [G₄S]₄ linker) (SEQ ID NO: 35)
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS GGGGSGGGGSGGGGSGGGGS
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
ID10A (ID1A with S91Y) (SEQ ID NO: 36)
DVQLQESGGGLVQPGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
ID12A (heterobihead of B4F10 and ID1A with [G₄S]₄ linker) (SEQ ID NO: 38)
DVQLQESGGGLVQAGGSLRLSCAASGRTSD VYAMG WFRQVPGKEREFVA TINRSGSDSYYADSVKG
RFTISRDNAKNTVYLQMNSLKPEETAVYYCAA SRSDCIGYGCRRVSQDY WGQGTQVTVSS GGGGSGGGGSGGGGSGGGGS
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
ID17A (ID12A with two alanine substitutions (R27A, P14A) and with [G₄S]₄ linker) (SEQ ID NO: 41)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SRSDCIGYGCRRVSQDY WGQGTQVTVSS GGGGSGGGGSGGGGSGGGGS
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
ID29A (B4F10 + Q34A3 with [G₄S]₄ linker) (SEQ ID NO: 42)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SRSDCIGYGCHRVSQDY WGQGTQVTVSS GGGGSGGGGSGGGGSGGGGS
DVQLQESGGGLVQAGGSLRLSCVISG MDFS HKPAG WFRQAPGKEREFVA SITTRASTHYADSVKG
RFTISRDNAKNTVYLEMNSLKPEDTAVYYCNS EYY WGQGTQVTVSS
ID30A (B4F10 + Q34A3 with [G₄S]₄ linker) (SEQ ID NO: 43)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SRSDCIGYGCRHVSQDY WGQGTQVTVSS GGGGSGGGGSGGGGSGGGGS
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
ID31A (B4F10 + Q34A3 with [G₄S]₄ linker) (SEQ ID NO: 44)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SRSDCIGYGCHHVSQDY WGQGTQVTVSS GGGGSGGGGSGGGGSGGGGS
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
ID32A (B4F10 + Q34A3 with [G₄S]₄ linker) (SEQ ID NO: 45)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SHSDCIGYGCRRVSQDY WGQGTQVTVSS GGGGSGGGGSGGGGSGGGGS
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
ID34A (B4F10 + Q34A3 with [G₄S]₄ linker) (SEQ ID NO: 47)
DVQLQESGGGLVQAGGSLRLSCAASGATSD VYAMG WFRQVPGKEREFVA TINRSGSDSYYADSVKG
RFTISRDNAKNTVYLQMNSLKPEETAVYYCAA SRSDCIGHGCHRVSQDY WGQGTQVTVSS GGGGSGGGGSGGGGSGGGGS
DVQLQESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS
ID11A (B14G12arm + ID1A with [G₄S]₄ linker) (SEQ ID NO: 37)
DVQLQESGGGLVQAGGSLRLSCAASGLSIS NYNMA WFRQAPGKERELVA VITWTGGSTYYAEDVKG
RLTISRDNAKNTVFLQMNSLKPEDTAIYFCAA GLKGSGRSLSYPDGYDN WGQGTQVTVSS GGGGSGGGGSGGGGSGGGGS
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
B14G12arm (SEQ ID NO: 16)
DVQLQESGGGLVQAGGSLRLSCAASGLSIS NYNMA WFRQAPGKERELVA VITWTGGSTYYAEDVKG
RLTISRDNAKNTVFLQMNSLKPEDTAIYFCAA GLKGSGRSLSYPDGYDN WGQGTQVTVSS
B14G12 (SEQ ID NO: 30)
QVQLQESGGGLVQAGGSLRLSCAASGLSIS NYNMA WFRQAPGKERELVA VITWTGGSTYYAEDVKG
RLTISRDNAKNTVFLQMNSLKPEDTAIYFCAA GLKGSGRSLSYPDGYDN WGQGTQVTVSS
ID13A (B14G3 + ID1A with [G₄S]₄ linker) (SEQ ID NO: 39)
DVQLQESGGGLVQAGGSLRLSCAASGHIFS RNAAG WFRQAPGKQREAVA TLSSGGNTTYRDSVKG
RFTISRDDTTNTVHLQMNSLKPEDTAVYYCNA MWY WGQGTQVTVSS GGGGSGGGGSGGGGSGGGGS
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
B14G3arm (SEQ ID NO: 17)
DVQLQESGGGLVQAGGSLRLSCAASGHIFS RNAAG WFRQAPGKQREAVA TLSSGGNTTYRDSVKG
RFTISRDDTTNTVHLQMNSLKPEDTAVYYCNA MWY WGQGTQVTVSS
B14G3 (SEQ ID NO: 31)
QVQLQESGGGLVQAGGSLRLSCAASGHIFS RNAAG WFRQAPGKQREAVA TLSSGGNTTYRDSVKG
RFTISRDDTTNTVHLQMNSLKPEDTAVYYCNA MWY WGQGTQVTVSS
ID15A (Q30A5 + ID1A with [G₄S]₄ linker) (SEQ ID NO: 40)
DVQLQESGGGLVQPGGSLKLSCAASGFTFG TYDMS WVRQAPGKRPEWVS AINPGGTSTYYADSVKG
RFTVSRDNAKDTLYLQMNSLKPEDTAVYYCTP SYASGIDYYPPPRDS WGQGTQVTVSS GGGGSGGGGSGGGGSGGGGS
DVQLQESGGGLVQPGGSLRLSCVISGMDFS HKPAG WFRQAPGKEREFVA SITTRASTHYADSVKG
RFTISRDNAKNTVYLEMNSLKPEDTAVYSCNS EYY WGQGTQVTVSS
Q30A5arm (SEQ ID NO: 18)
DVQLQESGGGLVQPGGSLKLSCAASGFTFG TYDMS WVRQAPGKRPEWVS AINPGGTSTYYADSVKG
RFTVSRDNAKDTLYLQMNSLKPEDTAVYYCTP SYASGIDYYPPPRDS WGQGTQVTVSS
Q30A5 (SEQ ID NO: 32)
RGAAGESGGGLVQPGGSLKLSCAASGFTFG TYDMS WVRQAPGKRPEWVS AINPGGTSTYYADSVKG
RFTVSRDNAKDTLYLQMNSLKPEDTAVYYCTP SYASGIDYYPPPRDS WGQGTQVTVSS
ID35A (ID33A with [G₄S]₄ linker with QSV substitutions in both Q34A3 and B4F10 arms) (SEQ ID NO: 48)
DVQLVESGGGLVQAGGSLRLSCAASGATSD VYAMG WFRQVPGKEREFVA TINRSGSDSYYADSVKG
RFTISRDNAKNTVYLQMNSLKPEETAVYYCAA SHSDCIGYGCHHVSQDY WGQGTQVTVSS GGGGSGGGGSGGGGSGGGGS
DVQLVESGGGLVQAGGSLRLSCVISGMDFS HKPAG WFRQAPGKEREFVA SITTRASTHYADSVKG
RFTISRDNAKNTVYLEMNSLKPEDTAVYYCNS EYY WGQGTQVTVSS

Suitably the polypeptide of the invention comprises an immunoglobulin chain variable domain which binds to Clostridium difficile toxin A, wherein the immunoglobulin chain variable domain comprises three complementarity determining regions (CDR1-CDR3) and four framework regions (FR1-FR4), wherein:

(a) CDR1 comprises a sequence sharing 60% or greater, such as 80% or greater sequence identity with SEQ ID NO: 1, CDR2 comprises a sequence sharing 60% or greater, such as 70% or greater, such as 75% or greater, such as 80% or greater, such as 85% or greater, such as 90% or greater sequence identity with SEQ ID NO: 2 and CDR3 comprises a sequence sharing 60% or greater, such as 70% or greater, such as 75% or greater, such as 80% or greater, such as 85% or greater, such as 90% or greater sequence identity with SEQ ID NO: 3; suitably any residues of CDR1, CDR2 or CDR3 differing from their corresponding residues in SEQ ID NO: 1, SEQ ID NO: 2 or SEQ ID NO: 3, respectively, are conservative substitutions with respect to their corresponding residues; suitably CDR3 is devoid of K or R; or

(b) CDR1 comprises a sequence sharing 60% or greater, such as 80% or greater sequence identity with SEQ ID NO: 4, CDR2 comprises a sequence sharing 60% or greater, such as 65% or greater, such as 70% or greater, such as 75% or greater, such as 80% or greater, such as 85% or greater, such as 90% or greater sequence identity with SEQ ID NO: 5 and CDR3 consists of a sequence having 2 to 4 residues and sharing 50% or greater, such as 75% or greater sequence identity with the sequence EYY; suitably any residues of CDR1, CDR2 or CDR3 differing from their corresponding residues in SEQ ID NO: 4, SEQ ID NO: 5 or the sequence EYY, respectively, are conservative substitutions with respect to their corresponding residues; suitably CDR3 consists of a sequence having 2 to 3 residues, 3 to 4 residues, more suitably 3 residues; or

(c) CDR1 comprises a sequence sharing 60% or greater, such as 80% or greater sequence identity with SEQ ID NO: 6, CDR2 comprises a sequence sharing 60% or greater, such as 70% or greater, such as 75% or greater, such as 80% or greater, such as 85% or greater, such as 90% or greater sequence identity with SEQ ID NO: 7 and CDR3 comprises a sequence sharing 60% or greater, such as 70% or greater, such as 75% or greater, such as 80% or greater, such as 85% or greater, such as 90% or greater sequence identity with SEQ ID NO: 8; suitably any residues of CDR1, CDR2 or CDR3 differing from their corresponding residues in SEQ ID NO: 6, SEQ ID NO: 7 or SEQ ID NO: 8, respectively, are conservative substitutions with respect to their corresponding residues; or

(d) CDR1 comprises a sequence sharing 60% or greater, such as 80% or greater sequence identity with SEQ ID NO: 9, CDR2 comprises a sequence sharing 60% or greater, such as 65% or greater, such as 70% or greater, such as 75% or greater, such as 80% or greater, such as 85% or greater, such as 90% or greater sequence identity with SEQ ID NO: 10 and CDR3 consists of a sequence having 2 to 4 residues and sharing 50% or greater, such as 75% or greater sequence identity with the sequence MWY; suitably any residues of CDR1, CDR2 or CDR3 differing from their corresponding residues in SEQ ID NO: 9, SEQ ID NO: 10 or the sequence MWY, respectively, are conservative substitutions with respect to their corresponding residues; suitably CDR3 consists of a sequence having 2 to 3 residues, 3 to 4 residues, more suitably 3 residues; or

(e) CDR1 comprises a sequence sharing 60% or greater, such as 80% or greater sequence identity with SEQ ID NO: 11, CDR2 comprises a sequence sharing 60% or greater, such as 70% or greater, such as 75% or greater, such as 80% or greater, such as 85% or greater, such as 90% or greater sequence identity with SEQ ID NO: 12 and CDR3 comprises a sequence sharing 65% or greater, such as 70% or greater, such as 75% or greater, such as 85% or greater, such as 90% or greater sequence identity with SEQ ID NO: 13; suitably any residues of CDR1, CDR2 or CDR3 differing from their corresponding residues in SEQ ID NO: 11, SEQ ID NO: 12 or SEQ ID NO: 13, respectively, are conservative substitutions with respect to their corresponding residues.

Suitably the polypeptide of the invention comprises an immunoglobulin chain variable domain which binds to Clostridium difficile toxin A, wherein the immunoglobulin chain variable domain comprises three complementarity determining regions (CDR1-CDR3) and four framework regions (FR1-FR4), wherein:

(a) CDR1 comprises or more suitably consists of SEQ ID NO: 1, CDR2 comprises or more suitably consists of SEQ ID NO: 2 and CDR3 comprises or more suitably consists of SEQ ID NO: 3; or

(b) CDR1 comprises or more suitably consists of SEQ ID NO: 4, CDR2 comprises or more suitably consists of SEQ ID NO: 5 and CDR3 consists of the sequence EYY; or

(c) CDR1 comprises or more suitably consists of SEQ ID NO: 6, CDR2 comprises or more suitably consists of SEQ ID NO: 7 and CDR3 comprises or more suitably consists of SEQ ID NO: 8; or

(d) CDR1 comprises or more suitably consists of SEQ ID NO: 9, CDR2 comprises or more suitably consists of SEQ ID NO: 10 and CDR3 consists of the sequence MWY; or

(e) CDR1 comprises or more suitably consists of SEQ ID NO: 11, CDR2 comprises or more suitably consists of SEQ ID NO: 12 and CDR3 comprises or more suitably consists of SEQ ID NO: 13.

Suitably the polypeptide of the invention comprises an immunoglobulin chain variable domain which binds to Clostridium difficile toxin A, wherein the immunoglobulin chain variable domain comprises three complementarity determining regions (CDR1-CDR3) and four framework regions (FR1-FR4), wherein:

(a) CDR1 comprises or more suitably consists of a sequence having no more than 3, more suitably no more than 2, more suitably no more than 1 addition(s), deletion(s) and/or substitutions(s) compared to SEQ ID NO: 1, CDR2 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 2 and CDR3 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 3; or

(b) CDR1 comprises or more suitably consists of a sequence having no more than 3, more suitably no more than 2, more suitably no more than 1 addition(s), deletion(s) and/or substitutions(s) compared to SEQ ID NO: 4, CDR2 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 5 and CDR3 comprises or more suitably consists of a sequence having no more than 2, more suitably no more than 1 addition(s), deletion(s) and/or substitutions(s) compared to the sequence EYY; or

(c) CDR1 comprises or more suitably consists of a sequence having no more than 3, more suitably no more than 2, more suitably no more than 1 addition(s), deletion(s) and/or substitutions(s) compared to SEQ ID NO: 6, CDR2 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 7 and CDR3 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 8; or

(d) CDR1 comprises or more suitably consists of a sequence having no more than 3, more suitably no more than 2, more suitably no more than 1 addition(s), deletion(s) and/or substitutions(s) compared to SEQ ID NO: 9, CDR2 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 10 and CDR3 comprises or more suitably consists of a sequence having no more than 2, more suitably no more than 1 addition(s), deletion(s) and/or substitutions(s) compared to the sequence MWY; or

(e) CDR1 comprises or more suitably consists of a sequence having no more than 3, more suitably no more than 2, more suitably no more than 1 addition(s), deletion(s) and/or substitutions(s) compared to SEQ ID NO: 11, CDR2 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 12 and CDR3 comprises or more suitably consists of a sequence having no more than 7, more suitably no more than 6, more suitably no more than 5, more suitably no more than 4, more suitably no more than 3, more suitably no more than 2, more suitably no more than 1 addition(s), deletion(s) and/or substitutions(s) compared to SEQ ID NO: 13.

Suitably the polypeptide of the invention comprises an immunoglobulin chain variable domain which binds to Clostridium difficile toxin A, wherein the immunoglobulin chain variable domain comprises three complementarity determining regions (CDR1-CDR3) and four framework regions (FR1-FR4), wherein CDR3 is devoid of K or R, more suitably CDR1, CDR2 and CDR3 are devoid of K or R. Suitably, CDR1, CDR2 and/or CDR3 have been mutated to replace one or more R or K residues with an H residue.

Suitably, (a) FR1, FR2, FR3 and FR4 each comprise a sequence sharing 40% or greater, such as 60% or greater, such as 70% or greater, such as 80% or greater, such as 90% or greater, such as 95% or greater sequence identity with FR1, FR2, FR3 and FR4 of SEQ ID NO 14, respectively; or

(b) FR1, FR2, FR3 and FR4 each comprise a sequence sharing 40% or greater, such as 60% or greater, such as 70% or greater, such as 80% or greater, such as 90% or greater, such as 95% or greater sequence identity with FR1, FR2, FR3 and FR4 of SEQ ID NO 15, respectively; or

(c) FR1, FR2, FR3 and FR4 each comprise a sequence sharing 40% or greater, such as 60% or greater, such as 70% or greater, such as 80% or greater, such as 90% or greater, such as 95% or greater sequence identity with FR1, FR2, FR3 and FR4 of SEQ ID NO 16, respectively; or

(d) FR1, FR2, FR3 and FR4 each comprise a sequence sharing 40% or greater, such as 60% or greater, such as 70% or greater, such as 80% or greater, such as 90% or greater, such as 95% or greater sequence identity with FR1, FR2, FR3 and FR4 of SEQ ID NO 17, respectively; or

(e) FR1, FR2, FR3 and FR4 each comprise a sequence sharing 40% or greater, such as 60% or greater, such as 70% or greater, such as 80% or greater, such as 90% or greater, such as 95% or greater sequence identity with FR1, FR2, FR3 and FR4 of SEQ ID NO 18, respectively.

Suitably FR1 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR1 of SEQ ID NO 14; FR2 of the polypeptide of the invention comprises or more suitably consist of a sequence having no more than 7, more suitably no more than 5, more suitably no more than 4, more suitably no more than 3, more suitably no more than 2, more suitably no more than 1 addition(s), deletion(s) and/or substitutions(s) compared to FR2 of SEQ ID NO 14; FR3 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR3 of SEQ ID NO 14; and FR4 of the polypeptide of the invention comprises or more suitably consist of a sequence having no more than 5, more suitably no more than 4, more suitably no more than 3, more suitably no more than 2, more suitably no more than 1 addition(s), deletion(s) and/or substitutions(s) compared to FR4 of SEQ ID NO 14.

Alternatively FR1 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR1 of SEQ ID NO 15; FR2 of the polypeptide of the invention comprises or more suitably consist of a sequence having no more than 7, more suitably no more than 5, more suitably no more than 4, more suitably no more than 3, more suitably no more than 2, more suitably no more than 1 addition(s), deletion(s) and/or substitutions(s) compared to FR2 of SEQ ID NO 15; FR3 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR3 of SEQ ID NO 15; and FR4 of the polypeptide of the invention comprises or more suitably consist of a sequence having no more than 5, more suitably no more than 4, more suitably no more than 3, more suitably no more than 2, more suitably no more than 1 addition(s), deletion(s) and/or substitutions(s) compared to FR4 of SEQ ID NO 15.

Alternatively FR1 of the polypeptide of the invention comprises or more suitably consist of a sequence having no more than 16, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR1 of SEQ ID NO 16; FR2 of the polypeptide of the invention comprises or more suitably consist of a sequence having no more than 7, more suitably no more than 5, more suitably no more than 4, more suitably no more than 3, more suitably no more than 2, more suitably no more than 1 addition(s), deletion(s) and/or substitutions(s) compared to FR2 of SEQ ID NO 16; FR3 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR3 of SEQ ID NO 16; and FR4 of the polypeptide of the invention comprises or more suitably consist of a sequence having no more than 5, more suitably no more than 4, more suitably no more than 3, more suitably no more than 2, more suitably no more than 1 addition(s), deletion(s) and/or substitutions(s) compared to FR4 of SEQ ID NO 16.

Alternatively FR1 of the polypeptide of the invention comprises or more suitably consist of a sequence having no more than 16, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR1 of SEQ ID NO 17; FR2 of the polypeptide of the invention comprises or more suitably consist of a sequence having no more than 7, more suitably no more than 5, more suitably no more than 4, more suitably no more than 3, more suitably no more than 2, more suitably no more than 1 addition(s), deletion(s) and/or substitutions(s) compared to FR2 of SEQ ID NO 17; FR3 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR3 of SEQ ID NO 17; and FR4 of the polypeptide of the invention comprises or more suitably consist of a sequence having no more than 5, more suitably no more than 4, more suitably no more than 3, more suitably no more than 2, more suitably no more than 1 addition(s), deletion(s) and/or substitutions(s) compared to FR4 of SEQ ID NO 17.

Alternatively FR1 of the polypeptide of the invention comprises or more suitably consist of a sequence having no more than 16, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR1 of SEQ ID NO 18; FR2 of the polypeptide of the invention comprises or more suitably consist of a sequence having no more than 7, more suitably no more than 5, more suitably no more than 4, more suitably no more than 3, more suitably no more than 2, more suitably no more than 1 addition(s), deletion(s) and/or substitutions(s) compared to FR2 of SEQ ID NO 18; FR3 of the polypeptide of the invention comprises or more suitably consist of a sequence having no more than 15, more suitably no more than 10, more suitably no more than 7, more suitably no more than 5, more suitably no more than 4, more suitably no more than 3, more suitably no more than 2, more suitably no more than 1 addition(s), deletion(s) and/or substitutions(s) compared to FR3 of SEQ ID NO 18; and FR4 of the polypeptide of the invention comprises or more suitably consist of a sequence having no more than 5, more suitably no more than 4, more suitably no more than 3, more suitably no more than 2, more suitably no more than 1 addition(s), deletion(s) and/or substitutions(s) compared to FR4 of SEQ ID NO 18.

Suitably residue 1 of FR1 is D, E or Q; and/or residue 5 of FR1 is V in the inventive polypeptide or each polypeptide of a multimeric construct.

Suitably the polypeptide of the invention comprises or more suitably consists of: SEQ ID NO: 14, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 15, SEQ ID NO: 29, SEQ ID NO: 33, SEQ ID NO: 34, SEQ ID NO: 36, SEQ ID NO: 16, SEQ ID NO: 30, SEQ ID NO: 17, SEQ ID NO: 31, SEQ ID NO: 18 or SEQ ID NO: 32.

Suitably the Clostridium difficile toxin A bound by the polypeptide of the invention comprises or more suitably consists of a sequence sharing 40% or greater, such as 60% or greater, such as 70% or greater, such as 80% or greater, such as 90% or greater, such as 95% or greater, such as 100% sequence identity with any one of SEQ ID NOs: 74-76.

### Linkers and Multimers

A construct according to the invention comprises multiple polypeptides and therefore may suitably be multivalent. Such a construct may comprise at least two identical polypeptides according to the invention. A construct consisting of two identical polypeptides according to the invention is a “homobihead”. In one aspect of the invention there is provided a construct comprising two or more identical polypeptides of the invention.

Alternatively, a construct may comprise a polypeptide of the invention and at least one further polypeptide which is different, but still a polypeptide according to the invention (a “heterobihead”). Suitably, the different polypeptide in such a construct is selected from: SEQ ID NO: 14, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 15, SEQ ID NO: 29, SEQ ID NO: 33, SEQ ID NO: 34, SEQ ID NO: 36, SEQ ID NO: 16, SEQ ID NO: 30, SEQ ID NO: 17, SEQ ID NO: 31, SEQ ID NO: 18 or SEQ ID NO: 32. Suitably such a construct is selected from SEQ ID NO: 35, SEQ ID NO: 37, SEQ ID NO: 38, SEQ ID

NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 and SEQ ID NO: 48.

Alternatively, such a construct may comprise (a) at least one polypeptide according to the invention and (b) at least one polypeptide such as an antibody or antigen-binding fragment thereof, which is not a polypeptide of the invention (also a “heterobihead”). The at least one polypeptide of (b) may bind TcdA (for example via a different epitope to that of (a)), or alternatively may bind to a target other than TcdA. Suitably the different polypeptide (b) binds to Clostridium difficile toxin B.

Constructs can be multivalent and/or multispecific. A multivalent construct (such as a bivalent construct) comprises two or more binding polypeptides therefore presents two or more sites at which attachment to one or more antigens can occur. An example of a multivalent construct could be a homobihead or a heterobihead. A multispecific construct (such as a bispecific construct) comprises two or more different binding polypeptides which present two or more sites at which either (a) attachment to two or more different antigens can occur or (b) attachment to two or more different epitopes on the same antigen can occur. An example of a multispecific construct could be a heterobihead. A multispecific construct is multivalent.

A construct of the invention may comprise, or more suitably consist of, one or more polypeptides according to the invention and suitably additionally comprise, or more suitably consist of, one, two, three, four, five, six, seven, eight, nine or more further polypeptides wherein each of the further polypeptides binds to a target, such as a target selected from the list consisting of Clostridium difficile toxin A and Clostridium difficile toxin B.

A construct consisting of a total of four polypeptides which each bind a target is known as a ‘quadrahead’. The format of such a construct according to the invention may be, from N- to C-terminal, suitably A-A-A-A, A-A-A-B, A-A-B-B, A-B-B-B, B-A-A-A, B-B-A-A, A-B-A-B, B-A-B-A, A-B-B-A or B-A-A-B, more suitably A-A-B-B, B-B-A-A, A-B-A-B, B-A-B-A, A-B-B-A or B-A-A-B, wherein A is a polypeptide which binds to Clostridium difficile toxin A and B is a polypeptide which binds to Clostridium difficile toxin B, wherein the polypeptides which bind to Clostridium difficile toxin A are identical or different and the polypeptides which bind to Clostridium difficile toxin B are identical or different.

Suitably the construct is of the format A-A′-B-B′, A-B-B′-A′ or B-A-A′-B′, wherein A is a polypeptide according to the invention, A′ is a different polypeptide according to the invention, B is a polypeptide which binds to Clostridium difficile toxin B and B′ is a different polypeptide which binds to Clostridium difficile toxin B. Suitably the construct is selected from: SEQ ID NO: 63-69.

Suitably, the polypeptides comprised within the construct are antibody fragments. More suitably, the polypeptides comprised within the construct are selected from the list consisting of: a VHH, a VH, a VL, a V-NAR, a Fab fragment and a F(ab′)2 fragment. More suitably, the polypeptides comprised within the construct are VHs or VHHs.

The polypeptides of the invention can be linked to each other directly (i.e. without use of a linker) or via a linker. The linker is suitably a polypeptide and will be selected so as to allow binding of the polypeptides to their epitopes. If used for therapeutic purposes, the linker is suitably non-immunogenic in the subject to which the polypeptides are administered. Suitably the linkers are of the format (G₄S)ₓ. Most suitably x is 6.

### Vectors and Hosts

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian and yeast vectors). Other vectors (e.g. non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g. replication defective retroviruses. adenoviruses and adeno-associated viruses), which serve equivalent functions, and also bacteriophage and phagemid systems. The invention also relates to nucleotide sequences that encode polypeptide sequences or multivalent and/or multispecific constructs. The term “recombinant host cell” (or simply “host cell”), as used herein, is intended to refer to a cell into which a recombinant expression vector has been introduced. Such terms are intended to refer not only to the particular subject cell but to the progeny of such a cell.

In one aspect of the invention there is provided a vector comprising the polynucleotide encoding the polypeptide or construct of the invention or cDNA comprising said polynucleotide. In a further aspect of the invention there is provided a host cell transformed with said vector, which is capable of expressing the polypeptide or construct of the invention. Suitably the host cell is a bacteria such as E. coli or a yeast such as a yeast belonging to the genera Aspergillus, Saccharomyces, Kluyveromyces, Hansenula or Pichia, such as Saccharomyces cerevisiae or Pichia pastoris.

### Stability

Suitably, the polypeptide or construct of the present invention substantially retains neutralisation ability and/or potency when delivered orally and after exposure to the intestinal tract (for example, after exposure to proteases present in the small and/or large intestine, C. difficile-specific proteases and inflammatory proteases). Such proteases include endopeptidase, trypsin and chymotrypsin. Proteases present in, or produced in, the stomach and small and/or large intestine include proteases sourced from intestinal commensal microflora and/or pathogenic bacteria, for example wherein the proteases are cell membrane-attached proteases, secreted proteases and proteases released on cell lysis. Most suitably the proteases are trypsin and chymotrypsin. Suitably the polypeptide or construct of the invention is substantially resistant to one or more proteases.

Suitably the intestinal tract is the intestinal tract of a human. The small intestine suitably consists of the duodenum, jejunum and ileum. The large intestine suitably consists of the cecum, colon, rectum and anal canal.

The polypeptide or construct of the present invention substantially retains neutralisation ability when suitably 10%, more suitably 20%, more suitably 30%, more suitably 40%, more suitably 50%, more suitably 60%, more suitably 70%, more suitably 80%, more suitably 90%, more suitably 95%, more suitably 100% of the original neutralisation ability of the polypeptide of the invention or construct is retained after exposure to proteases present in the small and/or large intestine such as trypsin or chymotrypsin.

Suitably the polypeptide or construct of the invention substantially retains neutralisation ability after exposure to proteases present in the small and/or large intestine such as trypsin or chymotrypsin for, for example, up to at least 15, more suitably up to at least 30, more suitably up to at least 45, more suitably up to at least 60 minutes at 37 degrees C.

Suitably 10% or more, more suitably 20% or more, more suitably 30% or more, more suitably 40% or more, more suitably 50% or more, more suitably 60% or more, more suitably 70% or more, more suitably 80% or more of the neutralisation ability of the polypeptide or construct of the invention is retained after up to 1 or more suitably up to 4 hours of exposure to conditions of the intestinal tract, more suitably the small or large intestine, more suitably human faecal supernatant.

### Therapeutic Use and Delivery

A therapeutically effective amount of a polypeptide, pharmaceutical composition or construct of the invention, is an amount which is effective, upon single or multiple dose administration to a subject, in neutralising TcdA to a significant extent in a subject. A therapeutically effective amount may vary according to factors such as the disease state, age, sex, and weight of the individual, and the ability of the polypeptide, pharmaceutical composition or construct to elicit a desired response in the individual. A therapeutically effective amount is also one in which any toxic or detrimental effects of the polypeptide of the invention, pharmaceutical composition or construct are outweighed by the therapeutically beneficial effects. The polypeptide or construct of the invention can be incorporated into pharmaceutical compositions suitable for administration to a subject. The polypeptide or construct of the invention can be in the form of a pharmaceutically acceptable salt.

A pharmaceutical composition of the invention may suitably be formulated for oral, intramuscular, subcutaneous, intravenous, intrarectal or enema delivery. The pharmaceutical compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. Solid dosage forms are preferred. The polypeptide of the invention, pharmaceutical composition or construct may be incorporated with excipients and used in the form of ingestible tablets, buccal tablets, troches, capsules, elixirs, suspensions, syrups, wafers, and the like.

Typically, the pharmaceutical composition comprises a polypeptide or construct of the invention and a pharmaceutically acceptable diluent or carrier. Examples of pharmaceutically acceptable carriers include one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the polypeptide or construct of the invention. Pharmaceutical compositions may include antiadherents, binders, coatings, disintegrants, flavours, colours, lubricants, sorbents, preservatives, sweeteners, freeze dry excipients (including lyoprotectants) or compression aids. Suitably, the polypeptide or construct of the invention is lyophilised before being incorporated into a pharmaceutical composition.

A polypeptide of the invention may also be provided with an enteric coating. An enteric coating is a polymer barrier applied on oral medication which protects the polypeptide from the low pH of the stomach. Materials used for enteric coatings include fatty acids, waxes, shellac, plastics, and plant fibers. Suitable enteric coating components include methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid. Suitable enteric coatings include pH-dependent release polymers. These are polymers which are insoluble at the highly acidic pH found in the stomach, but which dissolve rapidly at a less acidic pH. Thus, suitably, the enteric coating will not dissolve in the acidic juices of the stomach (pH ˜3), but will do so in the higher pH environment present in the small intestine (pH above 6) or in the colon (pH above 7.0). The pH-dependent release polymer is selected such that the polypeptide or construct of the invention will be released at about the time that the dosage reaches the small intestine.

A polypeptide, construct or pharmaceutical composition of the invention can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilisers, isotonic agents, suspending agents, emulsifying agents, stabilisers and preservatives. Acceptable carriers, excipients and/or stabilisers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as polysorbates, POE ethers, poloxamers, Triton-X, or polyethylene glycol.

For all modes of delivery, the polypeptide, pharmaceutical composition or construct of the invention may be formulated in a buffer, in order to stabilise the pH of the composition, at a concentration between 5-50, or more suitably 15-40 or more suitably 25-30 g/litre. Examples of suitable buffer components include physiological salts such as sodium citrate and/or citric acid. Suitably buffers contain 100-200, more suitably 125-175 mM physiological salts such as sodium chloride. Suitably the buffer is selected to have a pKa close to the pH of the composition or the physiological pH of the patient.

Exemplary polypeptide or construct concentrations in a pharmaceutical composition may range from about 1 mg/mL to about 200 mg/ml or from about 50 mg/mL to about 200 mg/mL, or from about 150 mg/mL to about 200 mg/mL.

An aqueous formulation of the polypeptide, construct or pharmaceutical composition of the invention may be prepared in a pH-buffered solution, e.g., at pH ranging from about 4.0 to about 7.0, or from about 5.0 to about 6.0, or alternatively about 5.5. Examples of suitable buffers include phosphate-, histidine-, citrate-, succinate-, acetate-buffers and other organic acid buffers. The buffer concentration can be from about 1 mM to about 100 mM, or from about 5 mM to about 50 mM, depending, for example, on the buffer and the desired tonicity of the formulation.

The tonicity of the pharmaceutical composition may be altered by including a tonicity modifier. Such tonicity modifiers can be charged or uncharged chemical species. Typical uncharged tonicity modifiers include sugars or sugar alcohols or other polyols, preferably trehalose, sucrose, mannitol, glycerol, 1,2-propanediol, raffinose, sorbitol or lactitol (especially trehalose, mannitol, glycerol or 1,2-propanediol). Typical charged tonicity modifiers include salts such as a combination of sodium, potassium or calcium ions, with chloride, sulfate, carbonate, sulfite, nitrate, lactate, succinate, acetate or maleate ions (especially sodium chloride or sodium sulphate); or amino acids such as arginine or histidine. Suitably, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 1 mM to 500 nM. Suitably, at least one isotonic agent is included in the composition.

A surfactant may also be added to the pharmaceutical composition to reduce aggregation of the formulated polypeptide or construct and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, and polysorbate 80. Exemplary concentrations of surfactant may range from about 0.001% to about 10% w/v.

A lyoprotectant may also be added in order to protect the polypeptide or construct of the invention against destabilizing conditions during the lyophilization process. For example, known lyoprotectants include sugars (including glucose, sucrose, mannose and trehalose); polyols (including mannitol, sorbitol and glycerol); and amino acids (including alanine, glycine and glutamic acid). Lyoprotectants can be included in an amount of about 10 mM to 500 mM.

The dosage ranges for administration of the polypeptide of the invention, pharmaceutical composition or construct of the invention are those to produce the desired therapeutic effect. The dosage range required depends on the precise nature of the polypeptide of the invention, pharmaceutical composition or construct, the route of administration, the nature of the formulation, the age of the patient, the nature, extent or severity of the patients condition, contraindications, if any, and the judgement of the attending physician. Variations in these dosage levels can be adjusted using standard empirical routines for optimisation.

Suitable daily dosages of polypeptide of the invention, pharmaceutical composition or construct of the invention are in the range of 50 ng-50 mg per kg, such as 50 ug-40 mg per kg, such as 5-30 mg per kg of body weight. The unit dosage can vary from less than 100 mg, but typically will be in the region of 250-2000 mg per dose, which may be administered daily or more frequently, for example 2, 3 or 4 times per day or less frequently for example every other day or once per week.

In one aspect of the invention there is provided the use of the polypeptide, pharmaceutical composition or construct of the invention in the manufacture of a medicament for the treatment of C. difficile infection. In a further aspect of the invention there is provided a method of treating C. difficile infection comprising administering to a person in need thereof a therapeutically effective amount of the polypeptide, pharmaceutical composition or construct of the invention.

The word ‘treatment’ is intended to embrace prophylaxis as well as therapeutic treatment. Treatment of infection also embraces treatment of exacerbations thereof and also embraces treatment of patients in remission from infection symptoms to prevent relapse of symptoms.

### Combination Therapy

A pharmaceutical composition of the invention may also comprise one or more active agents (e.g. active agents suitable for treating C. difficile infection). It is within the scope of the invention to use the pharmaceutical composition of the invention in therapeutic methods for the treatment of C. difficile infection as an adjunct to, or in conjunction with, other established therapies normally used in the treatment of C. difficile infection, such as antibiotics.

Possible combinations include combinations with, for example, one or more active agents selected from the list comprising C. difficile toxoid vaccine, ampicillin, amoxicillin, vancomycin, metronidazole, fidaxomicin, linezolid, nitazoxanide, rifaximin, ramoplanin, difimicin, clindamycin, cephalosporins (such as second and third generation cephalosporins), fluoroquinolones (such as gatifloxacin or moxifloxacin), macrolides (such as erythromycin, clarithromycin, azithromycin), penicillins, aminoglycosides, trimethoprim-sulfamethoxazole, chloramphenicol, tetracycline, imipenem, meropenem, antibacterial agents, bactericides, or bacteriostats. Possible combinations also include combinations with one or more active agents which are probiotics, for example Saccharomyces boulardii or Lactobacillus rhamnosus GG.

Hence another aspect of the invention provides a pharmaceutical composition of the invention in combination with one or more further active agents, for example one or more active agents described above.

In a further aspect of the invention, the polypeptide, pharmaceutical composition or construct is administered sequentially, simultaneously or separately with at least one active agent selected from the list above. In a further aspect of the invention, the polypeptide, pharmaceutical composition or construct is administered sequentially, simultaneously or separately with fecal microbiota transplantation (i.e. fecal bacteriotherapy, fecal transfusion, fecal transplant, stool transplant, fecal enema, human probiotic infusion).

Similarly, another aspect of the invention provides a combination product comprising:

(A) a polypeptide, pharmaceutical composition or construct of the present invention; and

(B) one or more other active agents,

wherein each of components (A) and (B) is formulated in admixture with a pharmaceutically-acceptable adjuvant, diluent or carrier. In this aspect of the invention, the combination product may be either a single (combination) formulation or a kit-of-parts. Thus, this aspect of the invention encompasses a combination formulation including a polypeptide, pharmaceutical composition or construct of the present invention and another therapeutic agent, in admixture with a pharmaceutically acceptable adjuvant, diluent or carrier.

The invention also encompasses a kit of parts comprising components:

(i) a polypeptide, pharmaceutical composition or construct of the present invention in admixture with a pharmaceutically acceptable adjuvant, diluent or carrier; and

(ii) a formulation including one or more other active agents, in admixture with a pharmaceutically-acceptable adjuvant, diluent or carrier, which components (i) and (ii) are each provided in a form that is suitable for administration in conjunction with the other.

Component (i) of the kit of parts is thus component (A) above in admixture with a pharmaceutically acceptable adjuvant, diluent or carrier. Similarly, component (ii) is component (B) above in admixture with a pharmaceutically acceptable adjuvant, diluent or carrier. The one or more other active agents (i.e. component (B) above) may be, for example, any of the agents mentioned above in connection with the treatment of C. difficile infection. If component (B) is more than one further active agent, these further active agents can be formulated with each other or formulated with component (A) or they may be formulated separately. In one embodiment component (B) is one other therapeutic agent. In another embodiment component (B) is two other therapeutic agents. The combination product (either a combined preparation or kit-of-parts) of this aspect of the invention may be used in the treatment or prevention of C. difficile infection.

Suitably the polypeptide, pharmaceutical composition or construct of the invention is for use as a medicament and more suitably for use in the treatment, prevention, diagnosis and/or detection of C. difficile infection, most suitably for use in the treatment of C. difficile infection.

### Preparative Methods

Polypeptides of the invention can be obtained and manipulated using the techniques disclosed for example in Green and Sambrook 2012 Molecular Cloning: A Laboratory Manual 4^{th} Edition Cold Spring Harbour Laboratory Press.

Monoclonal antibodies can be produced using hybridoma technology, by fusing a specific antibody-producing B cell with a myeloma (B cell cancer) cell that is selected for its ability to grow in tissue culture and for an absence of antibody chain synthesis (Köhler and Milstein 1975 Nature 256:495-497 and Nelson et al 2000 Molecular Pathology 53(3):111-117, herein incorporated by reference in their entirety).

A monoclonal antibody directed against a determined antigen can, for example, be obtained by:

a) immortalizing lymphocytes obtained from the peripheral blood of an animal previously immunized with a determined antigen, with an immortal cell and preferably with myeloma cells, in order to form a hybridoma,

b) culturing the immortalized cells (hybridoma) formed and recovering the cells producing the antibodies having the desired specificity.

Alternatively, the use of a hybridoma cell is not required. Accordingly, monoclonal antibodies can be obtained by a process comprising the steps of:

a) cloning into vectors, especially into phages and more particularly filamentous bacteriophages, DNA or cDNA sequences obtained from lymphocytes especially peripheral blood lymphocytes of an animal (suitably previously immunized with determined antigens),

b) transforming prokaryotic cells with the above vectors in conditions allowing the production of the antibodies,

c) selecting the antibodies by subjecting them to antigen-affinity selection,

d) recovering the antibodies having the desired specificity.

Methods for immunizing camelids, cloning the VHH repertoire of B cells circulating in blood (Chomezynnski and Sacchi 1987 Anal Biochem 162:156-159), and isolation of antigen-specific VHHs from immune (Arbabi-Ghahroudi et al 1997 FEBS Left 414:521-526) and nonimmune (Tanha et al 2002 J Immunol Methods 263:97-109) libraries using phage, yeast, or ribosome display are known (WO92/01047, Nguyen et al 2001 Adv Immunol 79:261-296 and Harmsen et al 2007 Appl Microbiol Biotechnol 77(1):13-22). These references are herein incorporated by reference in their entirety.

Antigen-binding fragments of antibodies such as the scFv and Fv fragments can be isolated and expressed in E. coli (Miethe et al 2013 J Biotech 163(2):105-111, Skerra et al 1988 Science 240(4855):1038-1041 and Ward et al Nature 1989 341:544-546, herein incorporated by reference in their entirety).

Mutations can be made to the DNA or cDNA that encode polypeptides which are silent as to the amino acid sequence of the polypeptide, but which provide preferred codons for translation in a particular host. The preferred codons for translation of a nucleic acid in, e.g., E. coli and S. cerevisiae, are known.

Mutation of polypeptides can be achieved for example by substitutions, additions or deletions to a nucleic acid encoding the polypeptide. The substitutions, additions or deletions to a nucleic acid encoding the polypeptide can be introduced by many methods, including for example error-prone PCR, shuffling, oligonucleotide-directed mutagenesis, assembly PCR, PCR mutagenesis, in vivo mutagenesis, cassette mutagenesis, recursive ensemble mutagenesis, exponential ensemble mutagenesis, site-specific mutagenesis (Ling et al 1997 Anal Biochem 254(2):157-178, herein incorporated by reference in its entirety), gene reassembly, Gene Site Saturation Mutagenesis (GSSM), synthetic ligation reassembly (SLR) or a combination of these methods. The modifications, additions or deletions to a nucleic acid can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, ensemble mutagenesis, chimeric nucleic acid multimer creation, or a combination thereof.

In particular, artificial gene synthesis may be used (Nambiar et al 1984 Science 223:1299-1301, Sakamar and Khorana 1988 Nucl. Acids Res 14:6361-6372, Wells et al 1985 Gene 34:315-323 and Grundstrom et al 1985 Nucl. Acids Res 13:3305-3316, herein incorporated by reference in their entirety). A gene encoding a polypeptide of the invention can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein 1998 Annu Rev Biochem 67:99-134)

Expression of immunoglobulin chain variable domains such as VHs and VHHs can be achieved using a suitable expression vector such as a prokaryotic cell such as bacteria, for example E. coli (for example according to the protocols disclosed in WO94/04678, which is incorporated herein by reference and detailed further below). Expression of immunoglobulin chain variable domains such as VHs and VHHs can also be achieved using eukaryotic cells, for example insect cells, CHO cells, Vero cells or suitably yeast cells such as yeasts belonging to the genera Saccharomyces, Kluyveromyces, Hansenula or Pichia. Suitably S. cerevisiae is used (for example according to the protocols disclosed in W094/025591, which is incorporated herein by reference and detailed further below).

Specifically, VHHs can be prepared according to the methods disclosed in WO94/04678 using E. coli cells by a process comprising the steps of:

a) cloning in a Bluescript vector (Agilent Technologies) a DNA or cDNA sequence coding for the VHH (for example obtained from lymphocytes of camelids or produced synthetically) optionally including a His-tag,

b) recovering the cloned fragment after amplification using a 5′ primer specific for the VHH containing an XhoI site and a 3′ primer containing the SpeI site having the sequence TC TTA ACT AGT GAG GAG ACG GTG ACC TG (SEQ ID NO: 49),

c) cloning the recovered fragment in phase in the Immuno PBS vector (Huse et al 1989Science 246 (4935):1275-1281, herein incorporated by reference in its entirety) after digestion of the vector with XhoI and SpeI restriction enzymes,

d) transforming host cells, especially E. coli by transfection with the recombinant Immuno PBS vector of step c,

e) recovering the expression product of the VHH coding sequence, for instance by affinity purification such as by chromatography on a column using Protein A, cation exchange, or a nickel-affinity resin if the VHH includes a His-tag.

Alternatively, immunoglobulin chain variable domains such as VHs and VHHs are obtainable by a process comprising the steps of:

a) obtaining a DNA or cDNA sequence coding for a VHH, having a determined specific antigen binding site,

b) amplifying the obtained DNA or cDNA, using a 5′ primer containing an initiation codon and a HindIII site, and a 3′ primer containing a termination codon having a XhoI site,

c) recombining the amplified DNA or cDNA into the HindIII (position 2650) and XhoI (position 4067) sites of a plasmid pMM984 (Merchlinsky et al 1983 J. Virol. 47:227-232, herein incorporated by reference in its entirety),

d) transfecting permissive cells especially NB-E cells (Faisst et al 1995 J Virol 69:4538-4543, herein incorporated by reference in its entirety) with the recombinant plasmid,

e) recovering the obtained products.

Further, immunoglobulin chain variable domains such as VHHs or VHs can be produced using E. coli or S. cerevisiae according to the methods disclosed in Frenken et al 2000 J Biotech 78:11-21 and WO99/23221 (herein incorporated by reference in their entirety) as follows:

After taking a blood sample from an immunised llama and enriching the lymphocyte population via Ficoll (a neutral, highly branched, high-mass, hydrophilic polysaccharide which dissolves readily in aqueous solutions—Pharmacia) discontinuous gradient centrifugation, isolating total RNA by acid guanidium thiocyanate extraction (Chomezynnski and Sacchi 1987 Anal Biochem 162:156-159), and first strand cDNA synthesis (e.g. using a cDNA kit such as RPN 1266 (Amersham)), DNA fragments encoding VHH and VH fragments and part of the short or long hinge region are amplified by PCR using the specific primers detailed on pages 22 and 23 of WO99/23221. Upon digestion of the PCR fragments with PstI and HindIII or BstEII, the DNA fragments with a length between about 300 and 450 bp are purified via agarose gel electrophoresis and ligated in the E. coli phagemid vector pUR4536 or the episomal S. cerevisiae expression vector pUR4548, respectively. pUR4536 is derived from pHEN (Hoogenboom et al 1991 Nucl Acid Res 19:4133-4137, herein incorporated by reference in its entirety) and contains the lacl^{q} gene and unique restriction sites to allow the cloning of the llama VHH and VH genes. pUR4548 is derived from pSY1 (Harmsen et al 1993 Gene 125:115-123, herein incorporated by reference in its entirety). From this plasmid, the BstEII site in the leu2 gene is removed via PCR and the cloning sites between the SUC2 signal sequence and the terminator are replaced in order to facilitate the cloning of the VH/VHH gene fragments. The VH/VHHs have the c-myc tag at the C-terminus for detection. Individual E. coli JM109 colonies are transferred to 96 well microtiter plates containing 150 ml 2TY medium supplemented with 1% glucose and 100 mg L⁻¹ ampicillin. After overnight growth (37 degrees C.), the plates are duplicated in 2TY medium containing 100 mg L⁻¹ ampicillin and 0.1 mM IPTG. After another overnight incubation and optionally freezing and thawing, cells are centrifuged and pelleted and the supernatant can be used in an ELISA. Individual S. cerevisiae colonies are transferred to test tubes containing selective minimal medium (comprising 0.7% yeast nitrogen base, 2% glucose, supplemented with the essential amino acids and bases) and are grown for 48 h at 30 degrees C. Subsequently, the cultures are diluted ten times in YPGaI medium (comprising 1% yeast extract, 2% bacto peptone and 5% galactose). After 24 and 48 h of growth, the cells are pelleted and the culture supernatant can be analysed in an ELISA. Absorbance at 600 nm (OD600) is optionally measured.

Further, immunoglobulin chain variable domains such as VH/VHHs can be produced using S. cerevisiae using the procedure as follows:

Isolate a naturally-occuring DNA sequence encoding the VH/VHH or obtain a synthetically produced DNA sequence encoding the VH/VHH, including a 5′-UTR, signal sequence, stop codons and flanked with SacI and HindII sites (such a synthetic sequence can be produced as outlined above or for example may be ordered from a commercial supplier such as Geneart (Life Technologies)).

Use the restriction sites for transfer of the VH/VHH gene to the multi-copy integration (MCI) vector pUR8569 or pUR8542, as follows. Cut the DNA sequence encoding the VHH optionally contained within a shuttle vector, cassette or other synthetic gene construct and the MCI vector with SacI and HindIII using: 25 ul VHH DNA (Geneart plasmid or MCI vector), 1 ul SacI, 1 ul HindIII, 3 ul of a suitable buffer for double digestion such as NEB buffer 1 (New England Biolabs) overnight at 37 degrees C. Run 25 ul of digested DNA encoding the VHH and 25 ul of digested MCI vector on a 1.5% agarose gel with 1×TAE buffer and then perform gel extraction for example using QIAquick Gel Extraction Kit (Qiagen)). Set-up a ligation of digested MCI vector and digested DNA encoding the VH/VHH as follows: 100 ng vector, 30 ng VHH gene, 1.5 ul 10×ligase buffer, 1 ul T4 DNA ligase, and ddH₂O. Then perform ligation overnight at 16 degrees C.

Next transform the E. coli cells. For chemical competent XL-1 blue cells, thaw 200 ul heat competent XL-1 blue cells and add 5 ul ligation mix on ice for about 30 minutes followed by heat shock for 90 seconds at 42 degrees C. Then add 800 ul Luria-Bertani low salt medium supplemented with 2% glucose and recover cells for 2 hours at 37 degrees C. Plate cells on Luria-Bertani agar and ampicillin (100 ug/ml) plates and keep overnight at 37 degrees C. For electro competent TG1 E. coli cells, use an electroporation cuvette. In the electroporation cuvette: thaw 50 ul electro competent TG1 cells and 1 ul ligation mix on ice for about 15 minutes. Place the cuvette in the holder and pulse. Add 500 ul of 2TY medium and recover cells for 30 minutes at 37 degrees C. Plate 100 ul of cells on Luria-Bertani, agar, containing ampicillin (100 ug/ml) and 2% glucose plates. Keep plates at 37 degrees C. overnight.

After cloning of the VH/VHH gene into E. coli as detailed above, S. cerevisiae can be transformed with the linearized MCI vector. Before transformation is carried out, some steps are performed: (i) the DNA should be changed from circular to linear by digestion or else the DNA cannot be integrated into the yeast genome and (ii) the digested DNA should be cleaned of impurities by ethanol precipitation. Also, during the transformation process, the yeast cells are made semi-permeable so the DNA can pass the membrane.

Preparation for yeast transformation: perform a HpaI digestion of the midi-prep prepared from the selected E. coli colony expressing the VH/VHH gene as follows. Prepare a 100 ul solution containing 20 ng of midi-prep, 5 ul HpaI, 10 ul of appropriate buffer such as NEB4 buffer (BioLabs), and ddH₂O.

Cut the DNA with the HpaI at room temperature overnight. Next perform an ethanol precipitation (and put to one side a 5 ul sample from HpaI digestion). Add 300 ul ethanol 100% to 95 ul HpaI digested midiprep, vortex, and spin at full speed for 5 minutes. Carefully decant when a pellet is present, add 100 ul of ethanol 70%, then spin again for 5 minutes at full speed. Decant the sample again, and keep at 50-60 degrees C. until the pellet is dry. Re-suspend the pellet in 50 ul ddH₂O. Run 5 ul on a gel beside the 5 ul HpaI digested sample.

Yeast transformation: prepare YNBglu plates. Use 10 g agar+425 ml water (sterilised), 25 ml filtered 20× YNB (3.35 g YNB (yeast nitrogen base) in 25 ml sterilized H₂O) and 50m1 sterile 20% glucose and pour into petri dishes. Pick one yeast colony from the masterplate and grow in 3 ml YPD (Yeast Extract Peptone Dextrose) overnight at 30 degrees C. Next day prepare about 600 ml YPD and use to fill 3 flasks with 275 ml, 225 ml and 100 ml YPD. Add 27.5 ul yeast YPD culture to the first flask and mix gently. Take 75 ml from the first flask and put this in the second flask, mix gently. Take 100 ml from the second flask and put in the third one, mix gently. Grow until reaching an OD660 of between 1 and 2. Divide the flask reaching this OD over 4 Falcon tubes, ±45 ml in each. Spin for 2 minutes at 4200 rpm. Discard the supernatant. Dissolve the pellets in two Falcon tubes with 45 l H₂O (reducing the number of tubes from 4 to 2). Spin for 2 minutes at 4200 rpm. Dissolve the pellets in 45 ml H₂O (from 2 tubes to 1). Spin for 2 minutes at 4200 rpm. Gently dissolve the pellets in 5 ml lithium acetate (LiAc) (100 mM), and spin for a few seconds. Carefully discard some LiAc, but retain over half of the LiAc in the tube. Vortex the cells, boil carrier DNA for 5 minutes and quickly chill in ice-water. Add to a 15 ml tube containing: 240 ul PEG, 50 ul cells, 36 uLiAc (1M), 25 ul carrier DNA, 45u1 ethanol precipitated VH/VHH. Mix gently after each step (treat the blank sample the same, only without ethanol precipitated VH/VHH). Incubate for 30 minutes at 30 degrees C., gently invert the tube 3-4 times, then heat shock for 20-25 minutes at 42 degrees C. Spin up to 6000rpm for a brief time. Gently remove the supernatant and add 250 ul ddH₂O and mix. Streak all of it on an YNBglu plate until plates are dry and grow for 4-5 days at 30 degrees C. Finally, prepare YNBglu plates by dividing plates in 6 equal parts, number the parts 1 to 6, inoculate the biggest colony and streak out number 1. Repeat for other colonies from big to small from 1 to 6. Grow at 30 degrees C. for 3-4 days large until colonies are produced. The VH/VHH clones are grown using glucose as a carbon source, and induction of VH/VHH expression is done by turning on the Galactose-7-promoter by adding 0.5% galactose. Perform a 3 mL small scale culture to test the colonies and choose which one shows the best expression of the VH or VHH. This colony is then used in purification.

Purification: the VH/VHH is purified by cation exchange chromatorgraphy with a strong anion resin (such as Capto S). On day 1, inoculate the selected yeast colony expressing the VH/VHH in 5 ml YPD medium (YP medium+2% glucose) and grow the cells in 25 mL sealed sterile tubes at 30 degrees C. overnight (shaking at 180 rpm). On day 2, dilute the 5 ml overnight culture in 50 mL freshly prepared YP medium+2% glucose+0.5% galactose, grow the cells in 250 ml aerated baffled flasks at 30 degrees C. for two nights (shaking at 180 rpm). On day 4, spin the cells down in a centrifuge at 4200 rpm for 20 min. Cation exchange purification step using a strong anion resin: adjust the pH of the supernatant containing the ligand to 3.5. Wash 0.75 ml resin (+/−0.5 mL slurry) per of 50 mL supernatant with 50 mL of ddH₂O followed by three washes with binding buffer. Add the washed resin to the supernatant and incubate the suspension at 4 degrees C. on a shaker for 1.5 hours. Pellet the resin-bound VH/VHH by centrifugation at 500 g for 2 minutes and wash it with wash buffer. Decant supernatant and re-suspend the resin with 10 mL of binding buffer. Put a filter in a PD-10 column, pour the resin in the column and let the resin settle for a while, then add a filter above the resin. Wait until all binding buffer has run through. Elute the VH/VHH with 6×0.5 ml elution buffer. Collect the elution fractions in eppendorf tubes. Measure the protein concentration of the 6 eluted fractions with a Nanodrop. Pool the fractions that contain the VHH and transfer the solution into a 3,500 Da cutoff dialysis membrane. Dialyze the purified protein solution against 3 L of PBS overnight at 4 degrees C. On day 5, dialyze the purified protein solution against 2 L of fresh PBS for an additional 2 hours at 4 degrees C. Finally, calculate the final concentration by BCA.

Although discussed in the context of the VH/VHH, the techniques described above could also be used for scFv, Fab, Fv and other antibody fragments if required.

Multiple antigen-binding fragments (suitably VH/VHHs) can be fused by chemical cross-linking by reacting amino acid residues with an organic derivatising agent such as described by Blattler et al Biochemistry 24:1517-1524 (herein incorporated by reference in its entirety). Alternatively, the antigen-binding fragments may be fused genetically at the DNA level i.e. a polynucleotide construct formed which encodes the complete polypeptide construct comprising one or more antigen-binding fragments. One way of joining multiple antigen-binding fragments via the genetic route is by linking the antigen-binding fragment coding sequences either directly or via a peptide linker. For example, the carboxy-terminal end of the first antigen-binding fragment may be linked to the amino-terminal end of the next antigen-binding fragment. This linking mode can be extended in order to link antigen-binding fragments for the construction of tri-, tetra-, etc. functional constructs. A method for producing multivalent (such as bivalent) VHH polypeptide constructs is disclosed in WO 96/34103 (herein incorporated by reference in its entirety).

Suitably, the polypeptide of the invention can be produced in a fungus such as a yeast (for example, S. cerevisiae) comprising growth of the fungus on a medium comprising a carbon source wherein 50-100 wt % of said carbon source is ethanol, according to the methods disclosed in WO02/48382. Large scale production of VHH fragments in S. cerevisiae is described in Thomassen et al 2002 Enzyme and Micro Tech 30:273-278 (herein incorporated by reference in its entirety).

In one aspect of the invention there is provided a process for the preparation of the polypeptide or construct of the invention comprising the following steps:

i) cloning into a vector, such as a plasmid, the polynucleotide of the invention,

ii) transforming a cell, such as a bacterial cell or a yeast cell capable of producing the polypeptide or construct of the invention, with said vector in conditions allowing the production of the polypeptide or construct,

iii) recovering the polypeptide or construct, such as by affinity chromatography.

The present invention will now be further described by means of the following non-limiting examples.

### EXAMPLES

### Example 1: Llama Immunisation

Llama immunisations were carried out using two different immunisation protocols to optimise the chance of obtaining potent cross-strain neutralising antibodies against TcdA.

Under the first protocol, two llamas were primed with 100 ug of TcdA toxoids prepared by formalin inactivation of purified TcdA from a C. difficile 027 strain, as well as with 10⁷ formalin inactivated spores from C. difficile strain 017 (M68) using Specol adjuvant. Llama 2 was boosted at 7, 14, 21, 28, 35 days with the same antigens, except that from day 14, gamma irradiated spores rather than formalin inactivated spores were used. In addition, the adjuvant was changed to IMS1312 for the last two boosts. Llama 1 had a similar immunisation protocol except that two further boosts were given on days 42 and 49. For llama 1, formalin inactivated spores were used on days 0, 7, 14 and 21, and Specol was the adjuvant. However, thereafter the adjuvant was changed to IMS1312 and gamma irradiated spores were used. Intramuscular injections were used for priming and boosting both llamas, except for the last boost which was administered subcutaneously. Blood samples were taken on days 28 (both llamas), 39 (llama 2, terminal sample), 42 (llama 1) and 53 (llama 1, terminal sample). On days 53 and 39 llamas 1 and 2 respectively were culled and lymph nodes removed. Lymphocytes were prepared from lymph nodes and blood samples to maximise the diversity of the immune response being sampled.

Under the second protocol, a llama was primed and boosted on days 14, 28 and 35 with 100 ug/injection of toxoids prepared by formalin inactivation from C. difficile 027 TcdA. Antigens were dissolved in 1 ml PBS +1 ml of Stimune and were injected intramuscularly. Blood samples were taken on days 0, 28 and 43. The llama was rested for 2.5 months then re-immunised three further times at 14 day intervals with TcdA toxoid prepared from C. difficile VPI 10463 TcdA. Two days after the final boost, blood was removed from the llama for lymphocyte preparation.

### Example 2: Phage Display, ICVD Selection and Production

RNA extracted from the llama lymphocytes was transcribed into cDNA using a reverse transcriptase kit. The cDNA was cleaned on PCR cleaning columns. IgH (both conventional and heavy chain) fragments were amplified using primers annealing at the leader sequence region and at the CH2 region. Two DNA fragments (˜700 bp and 900 bp) were amplified representing VHHs and VHs, respectively. The 700 bp fragment was cut from the gel and purified. A sample was used as a template for nested PCR. The amplified fragment was cleaned on a column and eluted. The eluted DNA was digested with BstEII and SfiI, and the 400 bp fragment was isolated from the gel. The fragments were ligated into the phagemid pUR8100 and transformed into E. coli TG1. Bacteria from overnight grown cultures of the libraries were collected and stored. The optical density at 600 nm (0D600) of these stocks was measured. The insert frequency was determined by picking multiple different clones from each of the library transformations and running colony PCR.

Phages were rescued from the bacteria containing libraries from the llamas by inoculating in medium containing glucose and ampicillin. When cultures were at log-phase, helper phage was added to infect the cultures and produce phages. Next day, produced phages were precipitated from the culture supernatant using a PEG/NaCl solution. The number of phages was determined by titration of the solution and infecting log-phase E. coli TG1 with the different phage dilutions. TcdA from ribotype 027 and strain VP110463 were coated into wells of maxisorb plates, overnight. The following amounts were used: 500 ng, 167 ng, 55 ng and 0 ng (non-coated well). Next day wells of the maxisorb plates were blocked with 4% Marvel in PBS, then phage from the libraries were added to the wells. After extensive washing with PBS-Tween and PBS, bound phages were eluted using alkaline pH shock and neutralized with 1 M Tris-HCl pH7.5. About half the eluted phages were rescued by infecting log-phase TG1 and selecting in medium containing ampicillin and glucose.

The sequences of the selected ICVDs are provided in Table 1A and Table 1B above. Selected ICVDs were B4F10, B1A7, B2H11, B3D5, B3H7, B3H11, B4F5, B4F7, B4G7, B4H7, Q34A3, B14G12, B14G3, and Q30A5.

Selected variable domains were subcloned from the phagemid vector into the expression plasmid pMEK222 (pMEK222 is a gene3 deleted version of the phagemid pUR8100, and where the cloned variable domain is followed by FLAG-6His tags, two stop codons and the M13 terminator sequence (see WO2013/064701)). The variable domain genes were digested with SfiI and Eco91I (or BstEII) and ligated into pMEK222 cut with the same restriction enzymes. E. coli strain BL21 DE3 was transformed by the ligations and plated on LB-agar plates supplemented with ampicillin and 2% glucose. Transformants were screened using colony PCR. Amplifications using the primers M13.rev (SEQ ID NO: 50) and M13.fw (SEQ ID NO: 51) led to the generation of plasmids containing inserts of 700 bp and of ˜350 bp (empty plasmids) observed by PCR.

Variable domains were produced from pMEK222 by inoculation of a fresh overnight grown culture at 1/100 dilution in 800 ml 2× YT, 0.1% glucose and 100 ug/ml ampicillin and grown for 2 h at 37 degrees C. Subsequently, 1 mM isopropyl beta-D-1-thiogalactopyranoside (IPTG) was added and the culture was grown for an additional 5 h at 37 degrees C. Bacteria were harvested by centrifugation and resuspended into 30 mL PBS. Bacteria were frozen by incubation at −20 degrees C. overnight. Bacteria were thawed at room temperature and fractionated by centrifugation. To the soluble fraction, which contains the variable domain, Co2+ agarose beads, for example, Talon resin (Thermo Scientific) were added to bind 6His-tagged variable domain. After washing the beads, bound variable domains were eluted with PBS supplemented with 150 mM imidazole. Finally, fractions containing the variable domains were dialyzed against PBS to remove the imidazole.

### Example 3: Primary Evaluation of ICVDs at Single Concentrations

B1A7, B2H11, B3D5, B3H7, B3H11, B4F5, B4F7, B4G7 and B4H7 are family members of

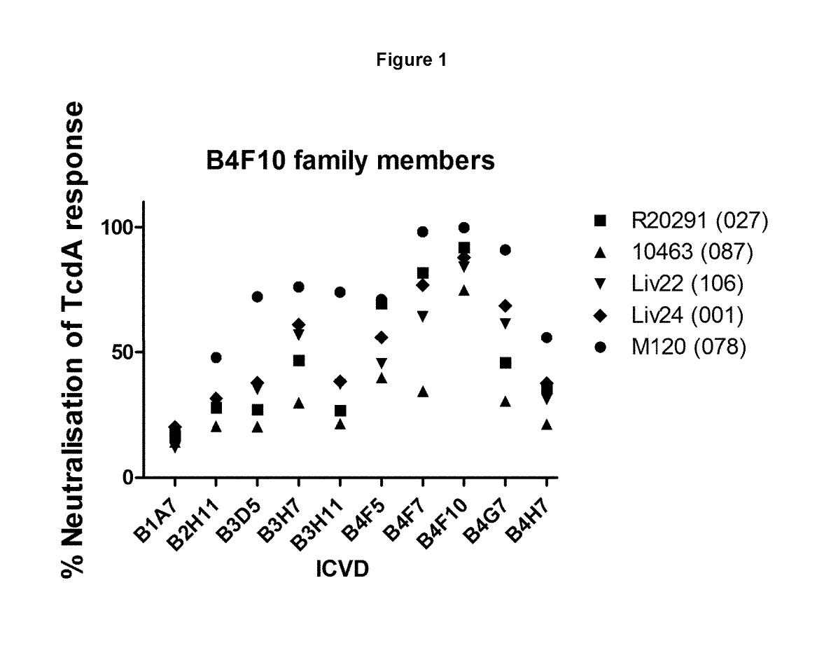

B4F10 and therefore share sequence similarity with one another (see Table 1B). The % neutralisation of TcdA ribotypes 027, 087, 106, 001 and 078 achieved by these B4F10 family members at single concentrations is shown in FIG. 1. It can be seen that many B4F10 family members achieve significant neutralisation across multiple TcdA ribotypes.

### Example 4: Modification of ICVDs

A series of modified anti-TcdA ICVDs were produced by yeast expression of DNA constructs (see Preparative Methods section, above). Homobiheads and heterobiheads were linked using a [Gly₄Ser]₄ amino acid linker. The modified anti-TcdA ICVDs were the following:

| | |
|---|---|
| ID1A | Q34A3 with E1D and V5Q |
| ID2A | ID1A with P14A |
| ID3A | Homobihead of ID1A |
| ID10A | ID1A with S91Y |
| ID11A | Heterobihead of B14G12 and ID1A |
| ID12A | Heterobihead of B4F10 and ID1A |
| ID13A | Heterobihead of B14G3 and ID1A |
| ID15A | Heterobihead of Q30A5 and ID1A |
| ID17A | ID12A with two alanine mutations (R27A, P14A) |

The sequences of these modified ICVDs are provided in Table 1A.

### Example 5: Determining the Potency of Unmodified Anti-TcdA ICVDs, Modified Anti-TcdA ICVDs and Anti-TcdA Constructs Against Multiple Ribotypes of TcdA Using the Standard Vero cell cytotoxicity assay.

### 5.1—Protocol for Preparing the Cytotoxicity Assay

### Culture and Maintenance of Vero Cells Prior to Use

Routine subculture of Vero cells can be achieved as follows:
1. Once a flask of cells has grown to full confluence, aspirate all cell culture medium and apply 2 ml 1x trypsin (dissolved in 0.02% EDTA, Sigma E8008). Once the trypsin has been applied work quickly to prevent loss of cells during washing.
2. Wash the first trypsin application over the surface of the cells and then fully aspirate to remove all traces of cell culture medium (any traces of serum from the medium will inhibit trypsin activity).
3. Apply 2 ml of trypsin and wash over the surface of the cells.
4. Remove approximately 1.5-1.7 ml of trypsin from the flask.
5. Tilt the flask so that the remaining 300-500 μL cover the Vero cells on the surface of the plate.
6. Incubate the cells at 37 ° C. 5% CO₂ for 10-12 minutes.
7. To stop trypsin activity add 10 ml Vero cell medium.
8. Resuspend the cells by gently jetting the suspension against the bottom of the flask with a pipette until the medium becomes cloudy (indicating dissipation of cell clumps). 3-4 times should be sufficient. Avoid excessive pipetting as this may harm the cells.
9. Add 0.2 to 0.5 ml of the cell suspension to 25-30m1 fresh Vero cell medium in a 75 cm² cell culture flask (Corning). Incubate the flask at 37 ° C. 5% CO₂ to allow growth of the cells to full confluence. This should occur in 3-5 days, depending on the inoculum volume and cell count. To obtain finer control over the process, cells may be enumerated using a haemocytometer, as outlined below, and added at a fixed inoculum to the medium. Once in a confluent state the cell monolayer should remain healthy for another 1-2 days without medium replacement. To prolong the life of the confluent monolayer for use it is often helpful to refresh ⅓-½ of the culture medium (do not replace all the medium as it will have been conditioned with cytokines from the growing Veros). The cells should be split before rounding and detachment starts to occur.

### Preparing Plates for the Vero Cell Cytotoxity Assay (Day-1)

Ideally, plates should be prepared the day before use in the cytotoxicity assay. However, plates may also be prepared on the day of use if necessary. If the latter is the case, prepare plates in the morning (for use in the afternoon) and ensure that at least 3 hours are allowed for cell attachment to the microplate prior to use. A fully confluent flask of Vero cells should be used to make the cell suspension for plating.
1. Add 150 μl sterile H₂O to the inter-well spaces and 300 μl to the top and bottom row of a 96-well flat bottomed microplate. This ensures that the cultured cells are hydrated during growth in the microplate.
2. Trypsinise and resuspend (in 10 ml Vero cell culture medium) a confluent flask of Vero cells, as described above.
3. Enumerate the cells using a haemocytometer and light microscope (take four independent counts and use the mean, for example using the four grid corners of a single haemocytometer slide). If there is any concern about cell viability following trypsinisation add Trypan blue dye to the cells before enumeration (1:1 v/v) and multiply the viable cell count ×2.
4. Dilute the cells to 5×10⁴ cells/ml in the required volume (allow 8 ml per assay plate) of Vero cell culture medium.
5. Using a multichannel pipette, dispense 100 μl of the cell suspension into each well. This is equivalent to 5000 cells/well. If multiple plates are being prepared keep swirling and/or pipetting the cell suspension between consecutive platings to ensure that the cells are evenly distributed.
6. Centrifuge the microplate at 1,000 rpm for 2 minutes at room temperature to fix the cells evenly in place across the bottom of the plate. Spin 2 plates maximum in each arm of the centrifuge to avoid the arms tipping inward and spilling the inter-well water.
7. Visually confirm that cell distribution and number are as expected using a light microscope.
8. Incubate plates at 37° C. 5% CO₂.

### Setting up the Assay (Day 0)

Note: All solutions described in this section are prepared in Vero cell culture medium. You should calculate the required final volume of each toxin and ICVD to cover the number of plates/combinations before starting the assay. Mix all solutions well (by vortexing and/or multiple inversions) between dilution steps.
1. Prepare the required volume of toxin at double (2×) the final assay concentration. The assay concentration required should be determined beforehand (see preliminary work, below).
2. Prepare the test ICVDs at double (2×) the top concentration to be tested in the assay. Aim for a top concentration of ICVD that will demonstrate a clear dose-response toxin neutralisation relationship in the assay (see example graph, below).
3. Prepare 10 serial dilutions (including the undiluted top concentration) of the 2× ICVD stock in a dilution trough. Typically, a ⅓ dilution produces a useful data range.
4. Use a 96-well round -bottom microplate to prepare mixed solutions before addition to the plates containing Vero cells.
5. In triplicate, prepare solutions of medium only, toxin only (1× dilution) and Triton-X100 (0.01%) controls and add each to empty plate wells.
6. Attach 10 μl pipette tips to the central 6 rows of an 8-channel aspirator. Carefully remove all medium (around 100 μl per well) from the Vero cell microplate prepared on Day 0.
7. Using a multichannel pipette, add 100 μl from one row of the preparation plate to the cells on the assay plate. Repeat this twice to fill the two adjacent rows on the assay plate (3 replicate rows in total):
8. Once plate feeding is complete incubate at 37° C. for 3 days.

### Processing the Assay (Day 3)

1. Observe the plates under a light microscope. Check for confluent growth in the medium only control wells and a good toxin response in the Toxin-only control well.
2. Using a multichannel pipette, in the dark, add 10 μl Alamar blue reagent (light sensitive) to each well.
3. Shake the plate for 30 seconds to ensure mixing of the Alamar blue into the culture medium.
4. Incubate the plate for 1 hr 30 minutes at 37° C. 5% CO₂
5. Following incubation, in the dark, add 50 μl 3% SDS.
6. Read the plate using a plate reader (such as Fluostar Omega), excitation filter 544, emission filter 590, bottom optic. Set the blank (against which the data will be corrected) to the three plate wells treated with Triton X100.
7. Calculate the mean of three replicates for each treatment on the plate. Calculate % toxin neutralisation values using the formula: % Neutralisation=(ICVD treatment−toxin control)*100/(medium control−toxin control).

### Preliminary Work: Determining the Optimal Amount of Toxin to Use in the Main Neutralisation Assay

For ease of interpretation in the main assay, the appropriate concentration of toxin to use should be determined beforehand by conducting a toxin dose-response experiment on Vero cells. Prepare 10 serial dilutions of toxin in a 12 well dilution trough. Use the remaining two wells for 0.01% Triton and a medium only control. Prepare a minimum of 330 μL of each solution in the dilution trough (this allows three replicates at 100 μl each). If there is no indication of how potent the toxin preparation is in advance, choose a broad dilution range for the preliminary experiment. This can be repeated over a finer concentration range, if necessary. Apply these solutions to Vero cells in a flat-bottomed microplate, incubate and process the plate as described above.

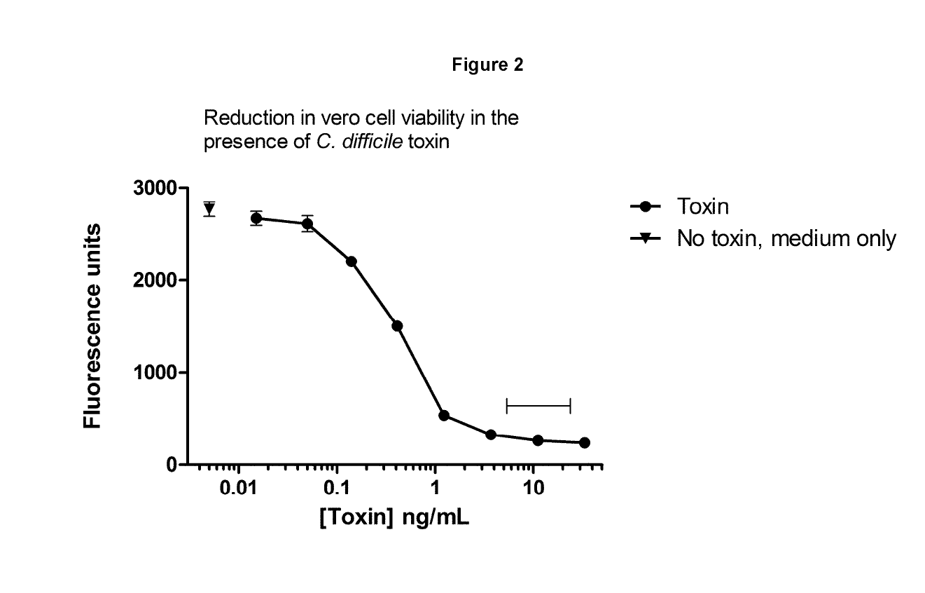

To assay an ICVD, or full antibody, for neutralisating activity against a given concentration of toxin, the minimum concentration of each toxin preparation capable of inducing the maximum reduction in cell viability is selected. An exemplary toxin dose-response curve on Vero cells is provided in FIG. 2. The horizontal bar indicates toxin concentrations suitable for use in the main neutralisation assay.

### 5.2—Potency Determination

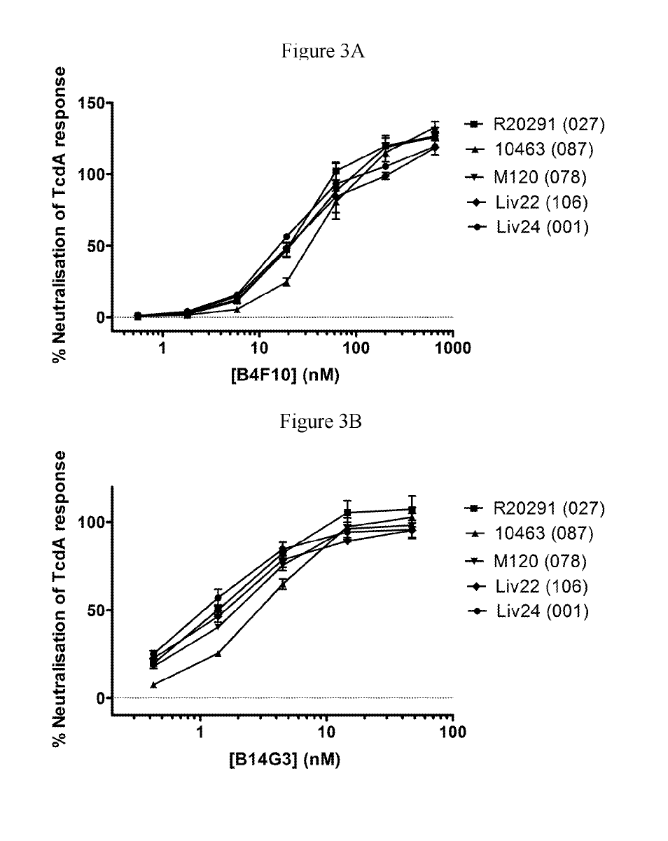

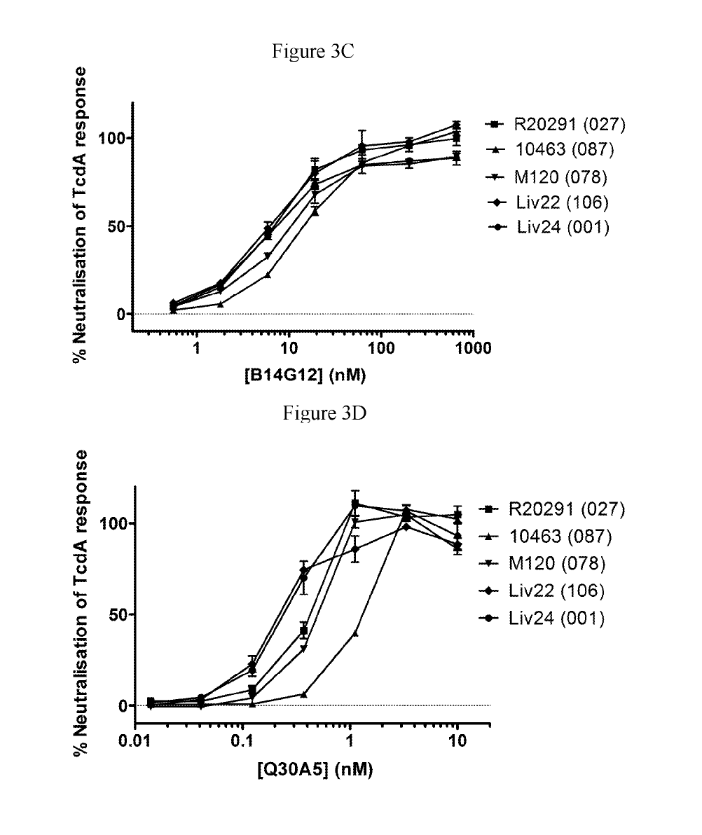

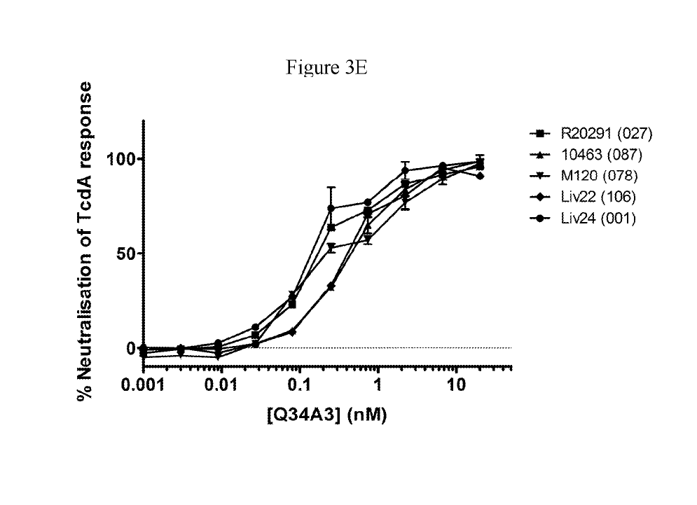

Standard Vero cell assays were carried out using the protocol detailed under 5.1 above. Neutralisation curves were produced (FIGS. 3-7) and IC50 values were generated in Graphpad prism using the % neutralisation data and ‘log(inhibitor) vs. response—Variable slope (four parameters)’ to fit curves and generate IC50s. In some cases, IC50 was calculated by interpolation of an entered 50% value using the fitted curve. The calculated IC50 values from the different assays were as follows:

TABLE 2

| | % TcdA Neutralisation IC50 (pM) | | | | |
|---|---|---|---|---|---|
| | R20291 | 10463 | M120 | | |
| | (027) | (087) | (078) | Liv22 (106) | Liv24 (001) |
| B4F10 | 25960 | 48900 | 31450 | 27570 | 20910 |
| B14G3 | 1407 | 2978 | 1919 | 1548 | 1100 |
| B14G12 | 6615 | 15093 | 10118 | 6264 | 7014 |
| Q30A5 | 412 | 1119 | 490 | 221 | 303 |
| Q34A3 | 271 | 601 | 3732 | 2016 | 1206 |
| ID1A | 255 | 634 | 3372 | 1502 | 1225 |
| ID2A | 311 | 699 | 3711 | 1615 | 1233 |
| ID3A | 199 | 385 | 2091 | 950 | 764 |
| ID10A | 255 | 767 | — | — | — |
| ID11A | 270 | 885 | — | — | — |
| ID12A | 2.62 | 6.53 | 1.94 | 6.18 | 2.75 |
| ID13A | 33 | 73 | — | — | — |
| ID15A | 139 | 266 | — | — | — |
| ID17A | 2.32 | 5.76 | 2.06 | 4.92 | 2.51 |

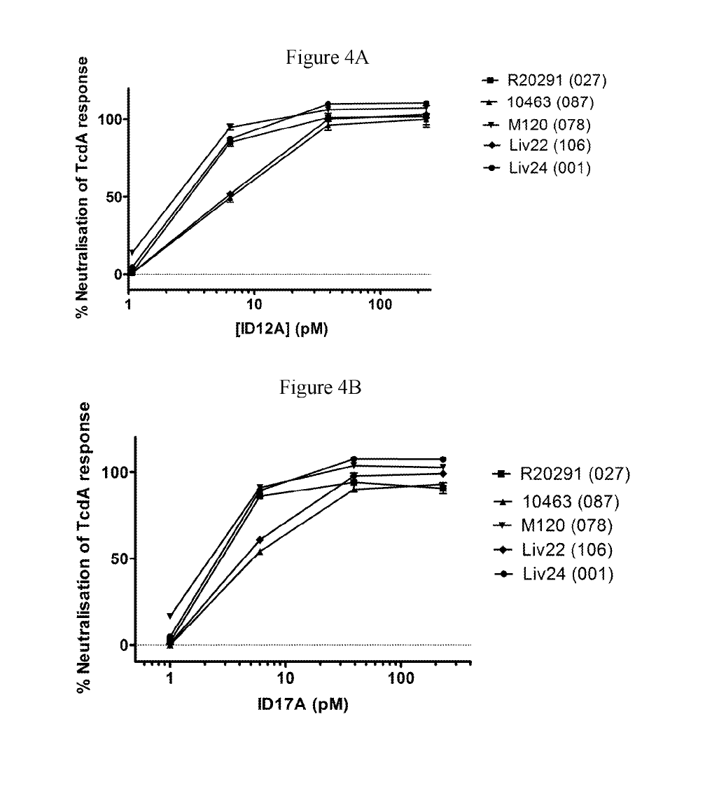

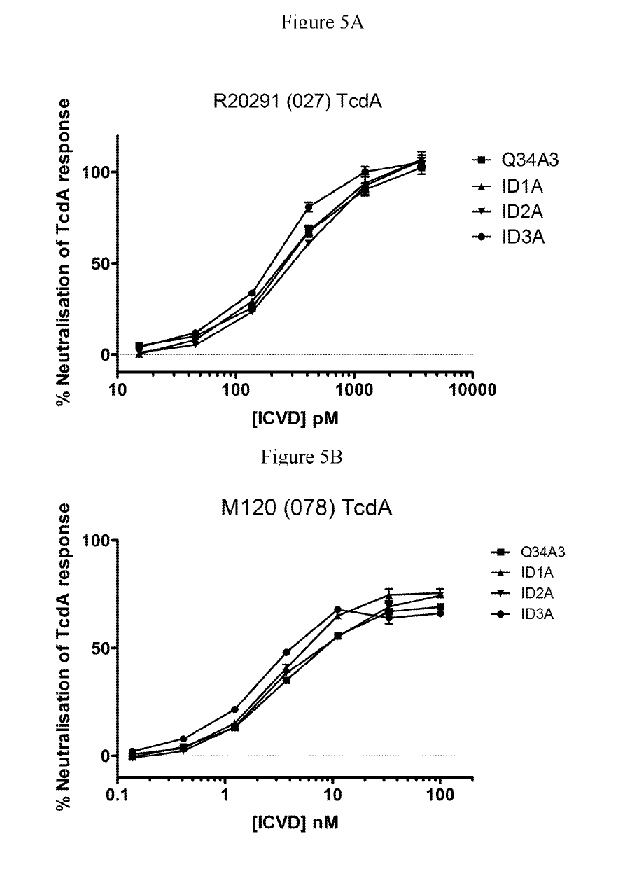

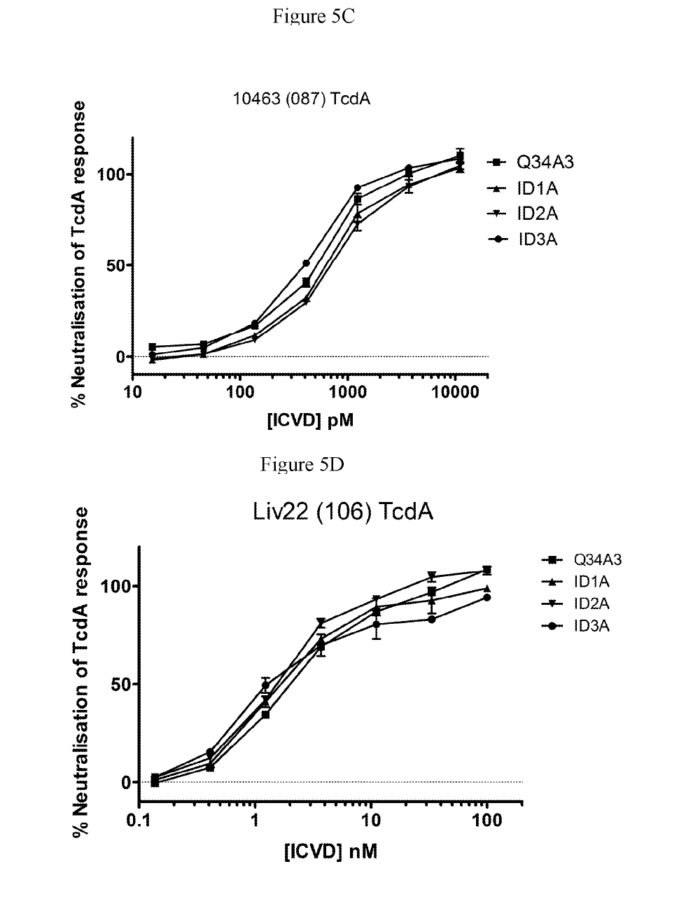

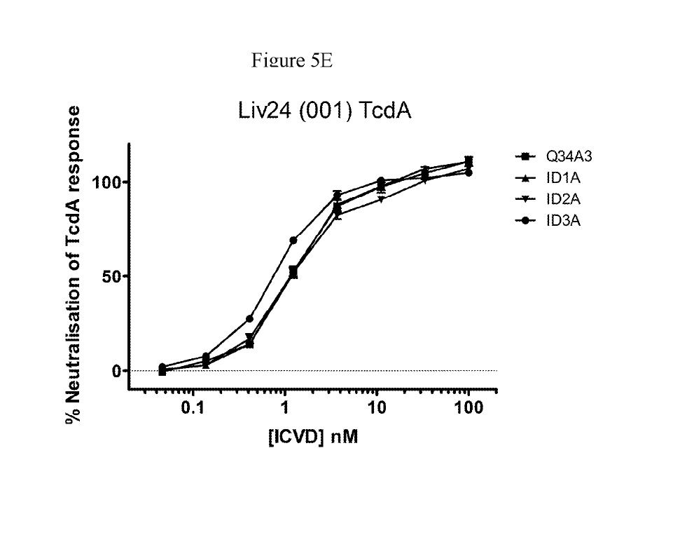

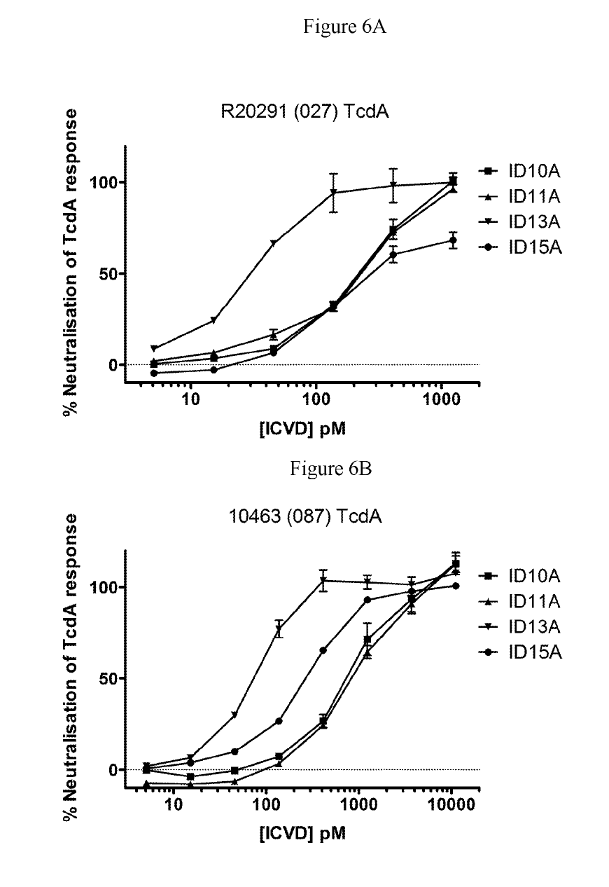

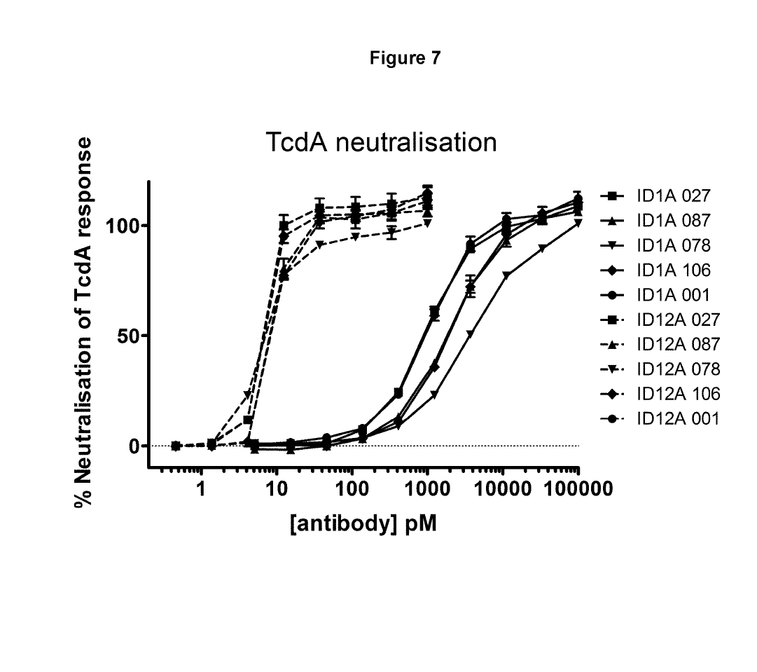

A hyphen denotes an assay which was not performed. It can be seen that most of the identified, unmodified ICVDs achieved high potency consistently against all TcdA ribotypes tested. The potency of Q30A5 against TcdA ribotype 087 was not quite as high as shown against other ribotypes (Table 2 and FIG. 3). The potencies of ID1A, ID2A and ID3A are substantially the same as Q34A3, indicating that none of the modifications resulted in a loss of potency (Table 2 and FIG. 5). The homobihead structure of ID3A resulted in no potency increase, suggesting no gain in avidity. ID12A was particularly potent against all TcdA ribotypes and ID17A remained highly potent against all TcdA ribotypes by comparison to ID12A (Table 2 and FIG. 4). ID10A-ID15A showed similar neutralisation profiles against both 027 and 087 TcdA (Table 2 and FIG. 6). Combining B14G12 with Q34A3 in bihead ID11A and combining Q30A5 with Q34A3 in heterobihead ID15A did nothing to improve neutralisation activity compared to Q34A3 alone. In contrast, heterobiheads ID12A and ID13A showed a marked increase in potency by comparison to ID1A (Table 2 and FIG. 7).

### Example 6: Comparison of Neutralising Capabilities of ID1A, ID12A and Monoclonal Antibody (mAb) 3D8

A standard Vero cell assay was conducted to assess the relative potencies of ID1A, ID12A and monoclonal antibody 3D8 (PCT Publication WO 2006/121422, in the name of University of Massachusetts and Medarex, Inc) against TcdA produced by multiple C. difficile ribotypes.

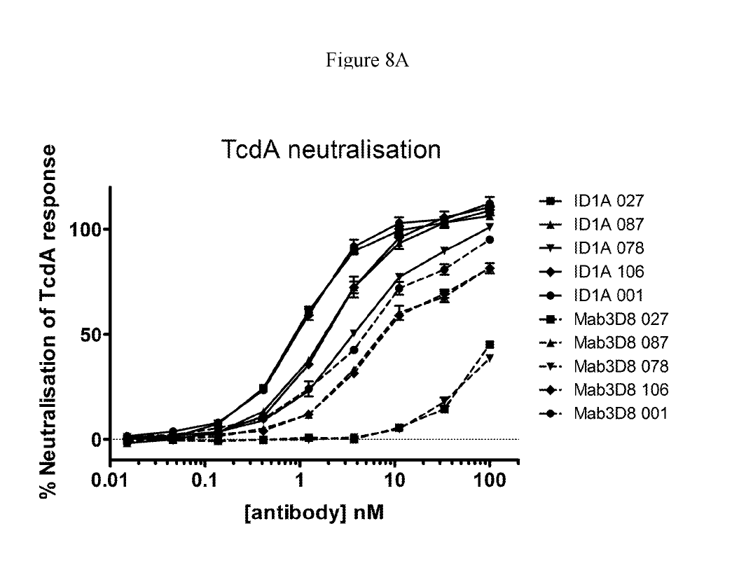

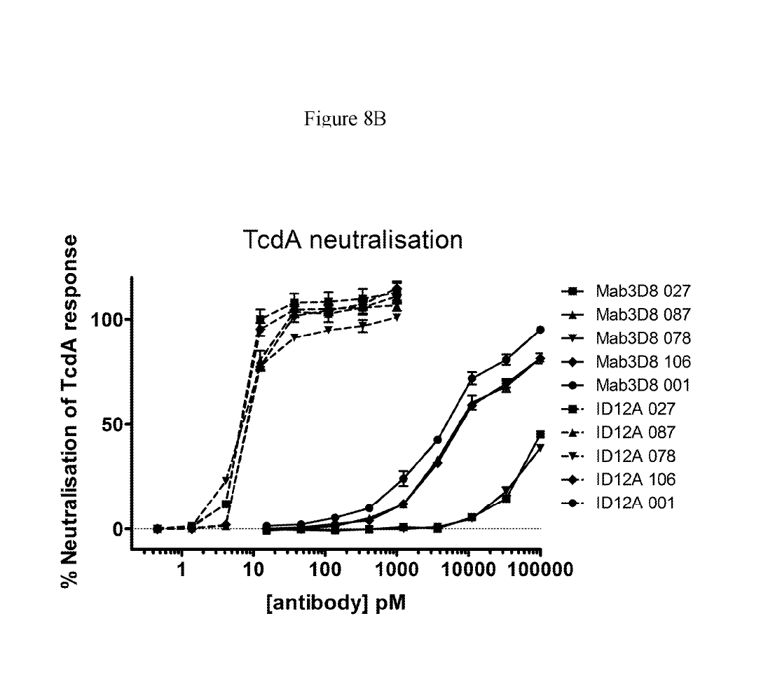

The results show that ID1A and ID12A are more potent than mAb 3D8 against all ribotypes of TcdA tested here using the standard Vero cell assay. The difference in potency between ID1A and ID12A with respect to mAb 3D8 was particularly marked when both were tested against TcdA from hypervirulent strain R20291 (027) (FIG. 8).

### Example 7: Further Modification of Constructs and Impact on Potency and Protease Stability

Genes encoding bihead constructs were designed based on ID17A in which R to H mutations (and a Y to H mutation) were introduced in CDR3 of the B4F10 arm of the bihead. These biheads were as follows:

| | |
|---|---|
| ID29A | ID17A with R in position 11 of CDR3 substituted with H. |
| ID30A | ID17A with R in position 12 of CDR3 substituted with H |
| ID31A | ID17A with R in positions 11 and 12 of CDR3 substituted with |
| | H |
| ID32A | ID17A with R in position 2 of CDR3 substituted with H |
| ID33A | ID17A with R in positions 2, 11 and 12 of CDR3 substituted |
| | with H |
| ID34A | ID17A with Y in position 8 of CDR3 substituted with H |

### 7.1—Impact on Potency

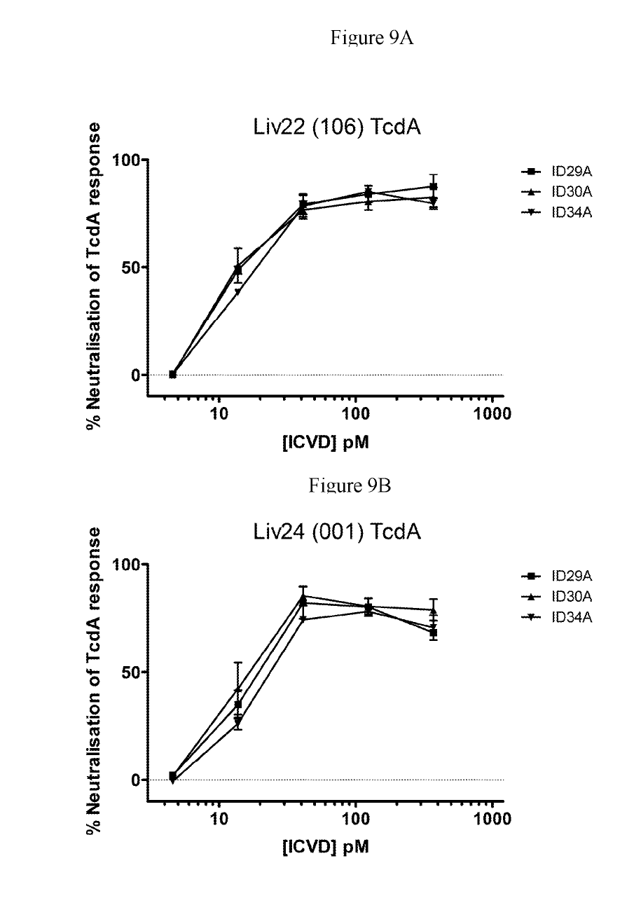

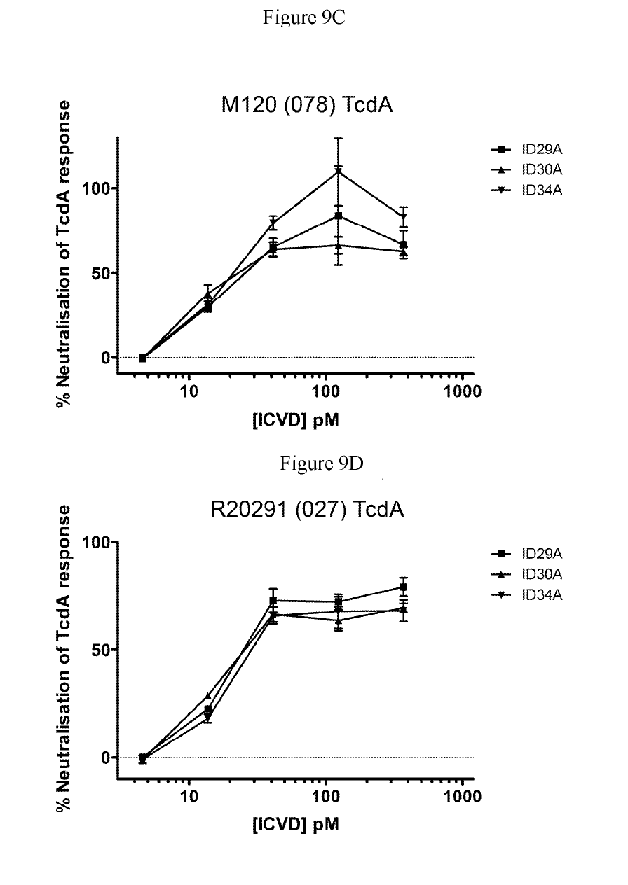

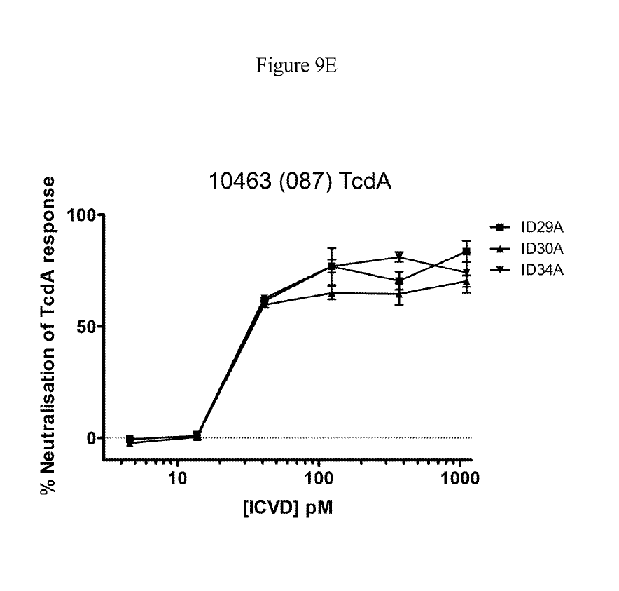

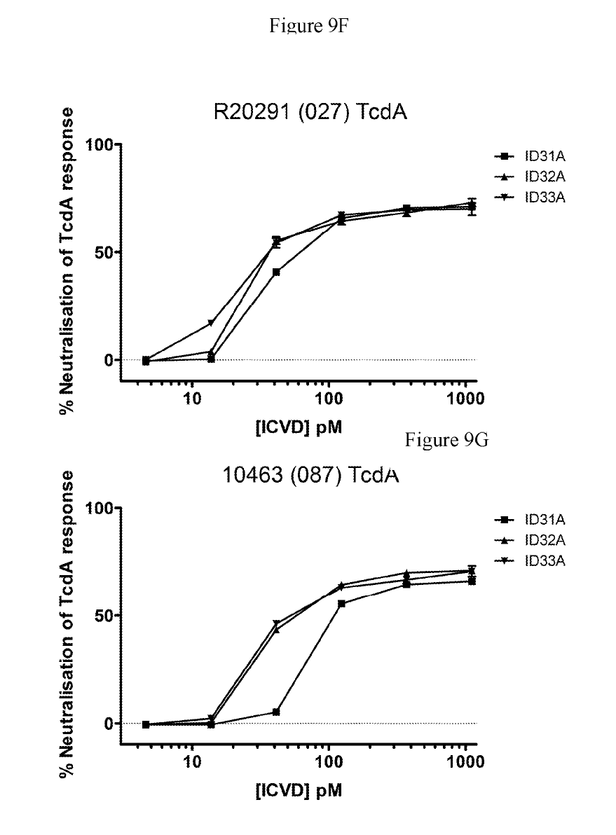

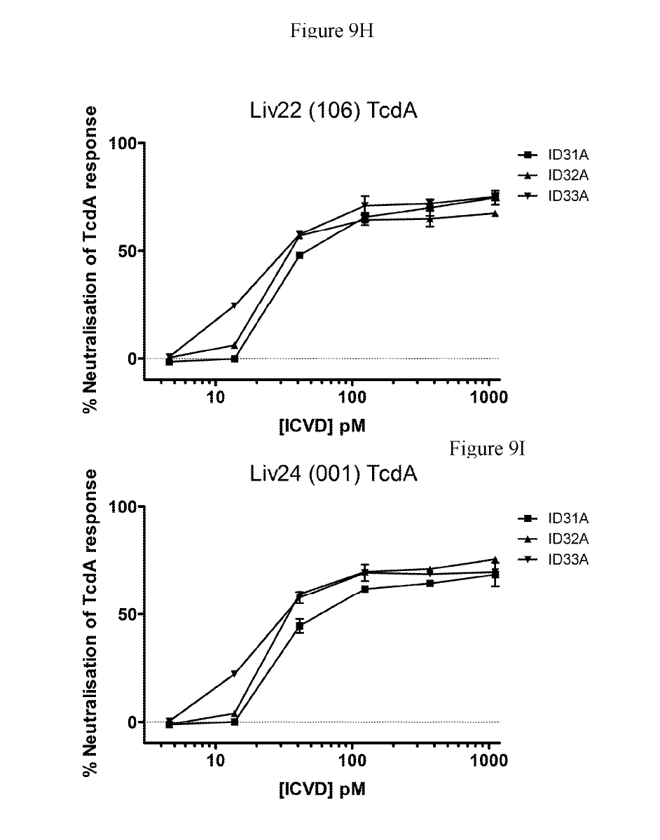

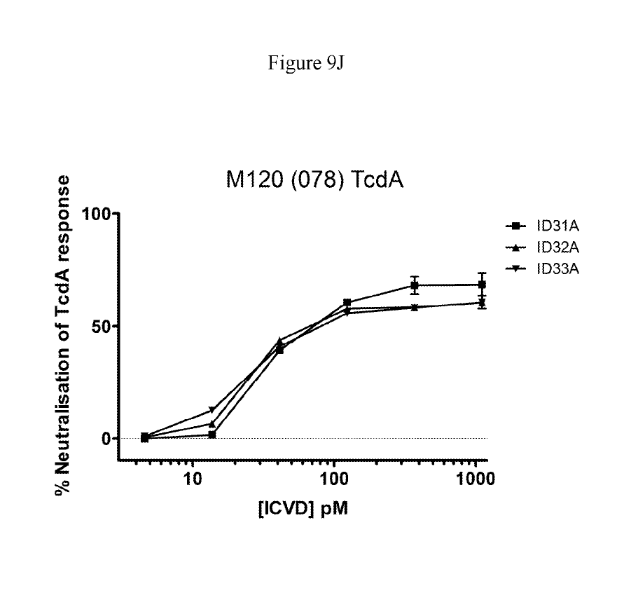

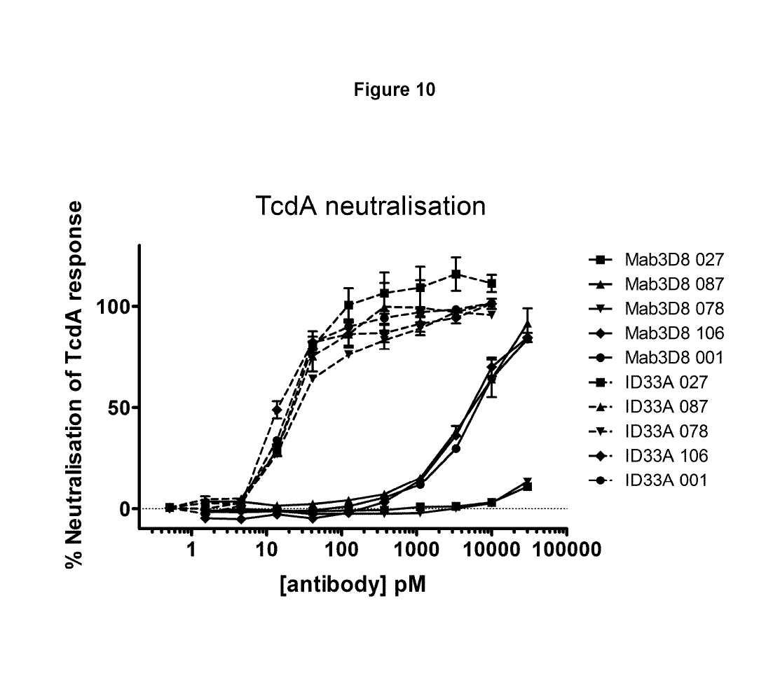

Neutralising activities of these biheads were measured using standard Vero cell assays (FIG. 9 and Table 3). These data show that ID29A-ID34A have similar potencies against all TcdA ribotypes tested, but ID31A is not as potent as the other biheads against TcdA ribotype 087. ID29A-ID34A potencies against the different TcdAs are approximately 10 fold less than ID17A. However, ID33A (and therefore presumably the other biheads) remain significantly more potent than mAb 3D8 according to these data (see, for example, FIG. 10).

TABLE 3

| | % TcdA Neutralisation IC50 (pM) | | | | |
|---|---|---|---|---|---|
| | R20291 | 10463 | M120 | | |
| | (027) | (087) | (078) | Liv22 (106) | Liv24 (001) |
| ID29A | 20.7 | 35.2 | 22 | 14.1 | 14.6 |
| ID30A | 16.8 | 33.1 | 17.1 | 13.5 | 13.9 |
| ID31A | 36.7 | 77.4 | 36.2 | 32.9 | 33.2 |
| ID32A | 27.7 | 34.8 | 28.6 | 25.2 | 27.2 |
| ID33A | 22.2 | 32.1 | 26.3 | 18.1 | 18.5 |
| ID34A | 22.6 | 34.7 | 19.6 | 16.6 | 17 |

### 7.2—Impact on Protease Stability—Exposure to Trypsin and Chymotrypsin

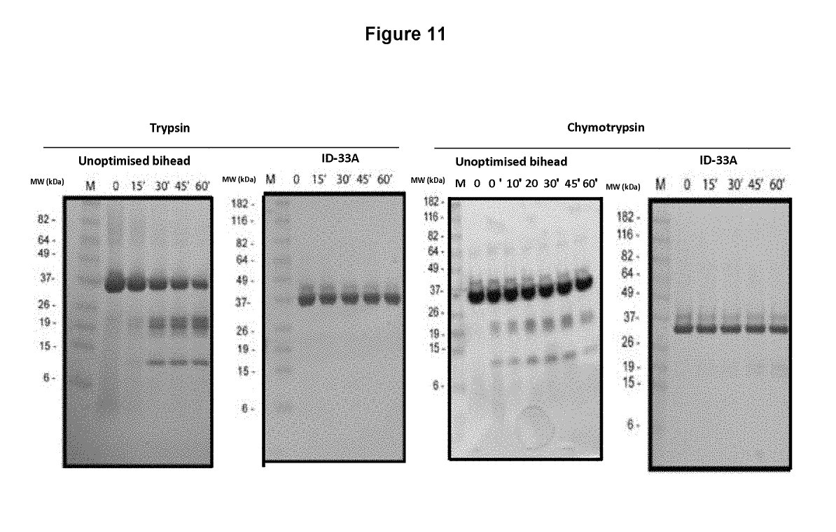

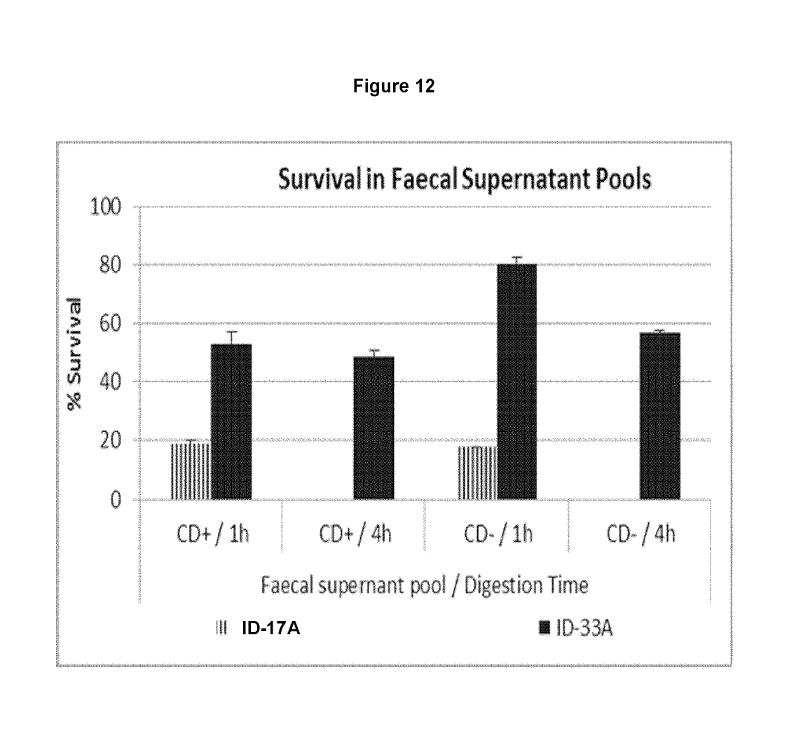

ID33A and ID17A were incubated with trypsin or chymotrypsin beads for intervals of 0 (untreated control), 15, 30, 45 and 60 minutes. These data demonstrate that ID17A bihead was digested by trypsin after between 15 and 30 minutes and cleaved by chymotrypsin before 10 minutes. ID33A however remained undigested (FIG. 11). Engineered bi-head construct ID33A therefore has substantial stability against trypsin and chymotrypsin.

7.3—Impact on Protease Stability—Exposure to Faecal Supernatant

ID33A and ID17A constructs were incubated in faecal supernatants. The faecal supernatant pools were each produced from 5 faecal samples from either C. difficile positive patients (CD+) or C. difficile negative patients (CD−). The constructs were digested in the supernatants for 1 hour or as a separate experiment for 4 hours and then measured using an ELISA. ID33A showed much greater % survival than ID17A over both time periods. Engineered bi-head construct ID33A therefore has substantial stability in faecal supernatant. The % survival was calculated by dividing the average variable domain concentration for a single time point by the average construct concentration in the 0-time point wells.

### Example 8: Construction and Potency Analysis of Quadrahead Constructs

Quadrahead constructs comprising two different anti-TcdA ICVDs (based on Q34A3 and B4F10) and two different anti-TcdB ICVDs (based on Q31B1 and B10F1) were produced in yeast using the methodology detailed in the Preparative Methods section above. Each ICVD in each quadrahead was connected by a [Gly₄Ser]₄ linker. The format of these quadraheads is summarised in Table 4 below:

TABLE 4

| | | | | | | | Position of CDR3 R to H | | | |
|---|---|---|---|---|---|---|---|---|---|---|
| | | | ICVD monomer order | | | | substitutions in each ICVD monomer | | | |
| | | | (N to C terminal) | | | | from N to C terminal | | | |
| Name | Format | Production (mg/ml) | 1st | 2nd | 3rd | 4th | 1st | 2nd | 3rd | 4th |
| ID-1C | AA′BB′ | 0.93 | B4F10 | Q34A3 | Q31B1 | B10F1 | | | | |
| ID-3C | BAA′B′ | 2.26 | Q31B1 | B4F10 | Q34A3 | B10F1 | | | | |
| ID-5C | BB′AA′ | 1.27 | Q31B1 | B10F1 | B4F10 | Q34A3 | 107 | 105 | | |
| ID-6C | ABB′A′ | 1.08 | B4F10 | Q31B1 | B10F1 | Q34A3 | | 107 | 105 | |
| ID-7C | A′BB′A | 1.22 | Q34A3 | Q31B1 | B10F1 | B4F10 | | 107 | 105 | |
| ID-8C | BB′AA′ | 0.33* (batch 1) | Q31B1 | B10F1 | B4F10 | Q34A3 | 107 | 105, | 100, 109, | |
| | | 0.72* (batch 2) | | | | | | 108 | 110 | |
| ID-11C | A′BB′A | 0.59* | Q34A3 | Q31B1 | B10F1 | B4F10 | | 107 | 105, 108 | 100, 109, |
| | | | | | | | | | | 110 |

*These quadraheads had a lower production level than ID-1C to ID-7C. However, it is expected this is due to batch to batch variation and that these quadraheads would normally be produced at a similar level to ID-1C to ID-7C.

It can be seen from Table 4 above that the order of ICVDs in a quadrahead influences production level, with ID-3C being produced at the highest level in yeast. ID-8C is ID-5C with additional CDR3 R to H substitutions. ID-11C is ID-7C with additional CDR3 R to H substitutions.

The potency of these quadraheads against TcdA and TcdB from various C. difficile ribotypes was analysed using the cytotoxicity assay described in Example 5 above.

### 8.1—Potency of ID-1 C (AA′BB') and ID-3C (BAA′B′)

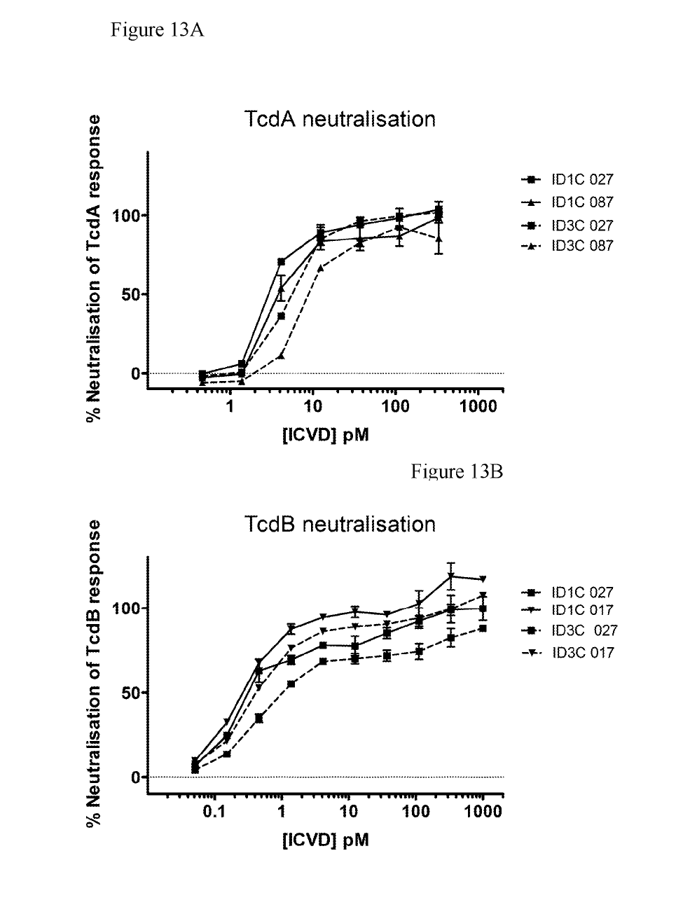

ID-1C and ID-3C were found to potently neutralise TcdA from ribotypes 027 and 087 (FIG. 13, Graph I) and TcdB from ribotypes 027 and 017 (FIG. 13, Graph II). The most significant difference between these two quadraheads is the separation between the two anti-TcdB ICVDs. In ID-1C, the separation is the [Gly₄Ser]₄ linker alone, whilst in ID-3C the two anti-TcdB ICVDs are separated by [Gly₄Ser]₄, anti-TcdA ICVD, [Gly₄Ser]₄, anti-TcdA ICVD, [Gly₄Ser]₄. This may be the reason for ID-1C having a slightly greater potency than ID-3C against TcdA and TcdB from these ribotypes.

### 8.2—Potency of ID-5C and ID-8C (BB′AA′)

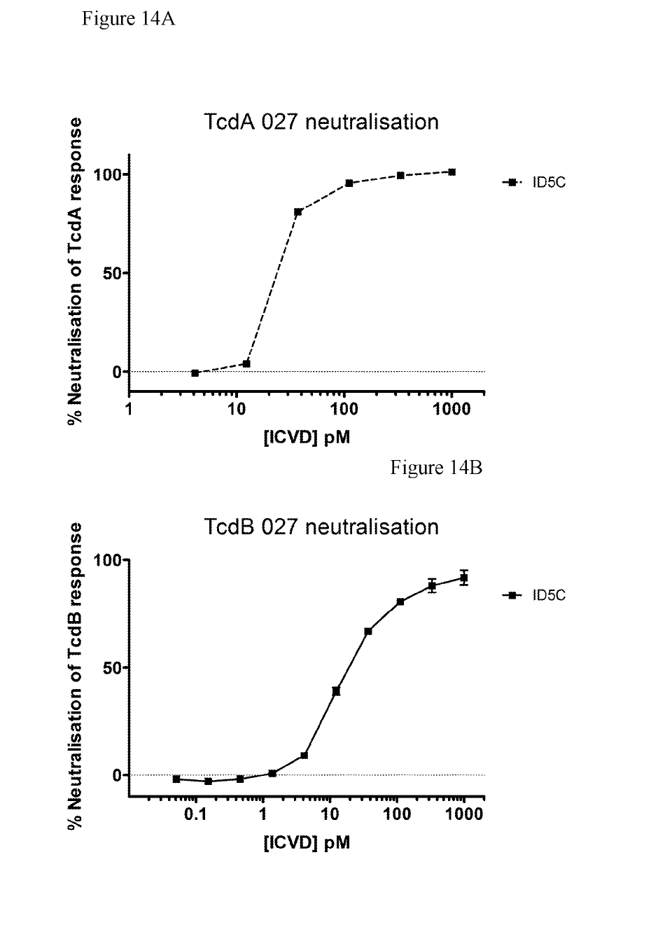

ID-5C was found to potently neutralise 027 TcdA (FIG. 14, Graph I) and 027 TcdB (FIG. 14, Graph II).

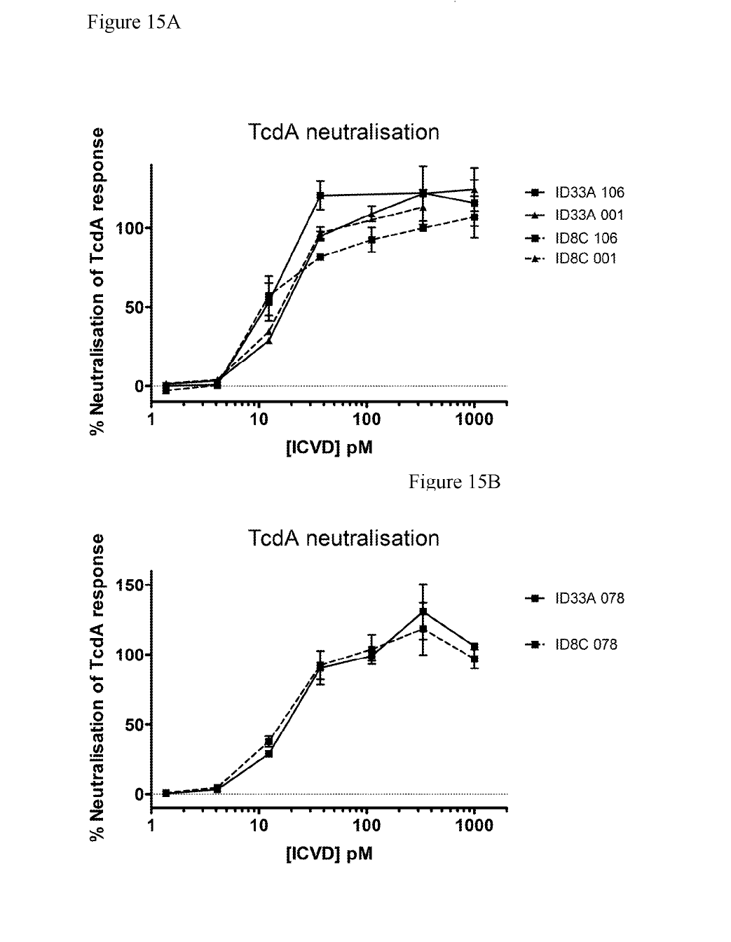

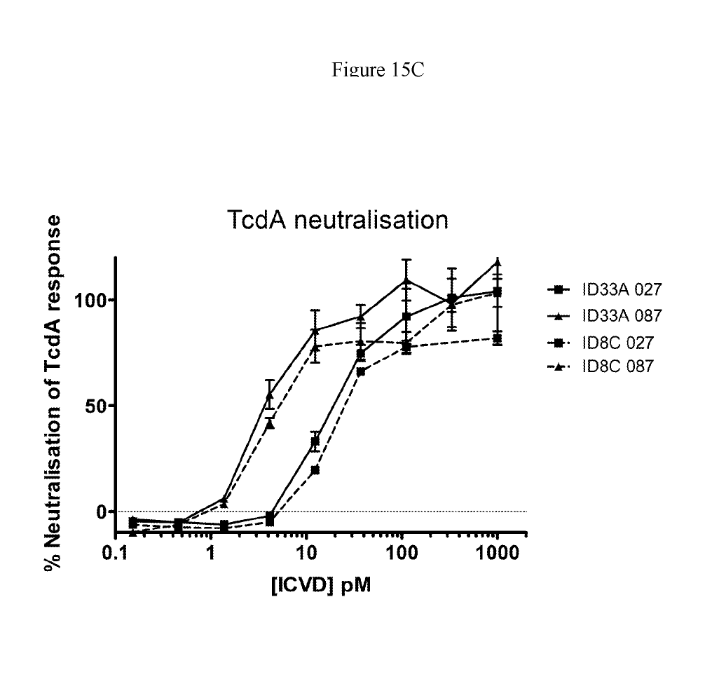

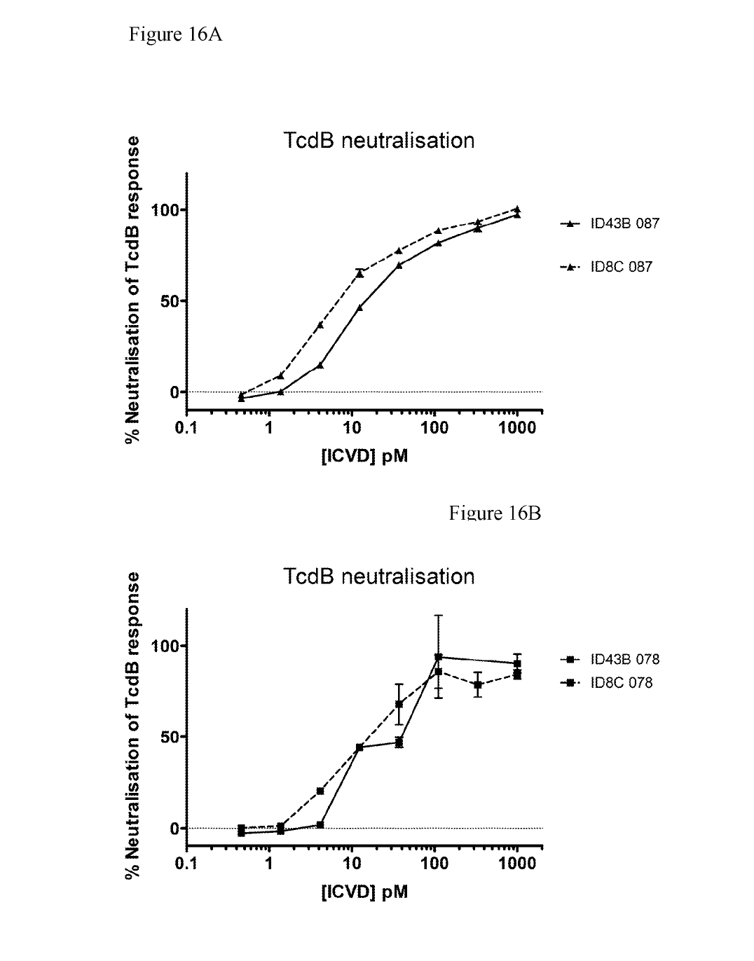

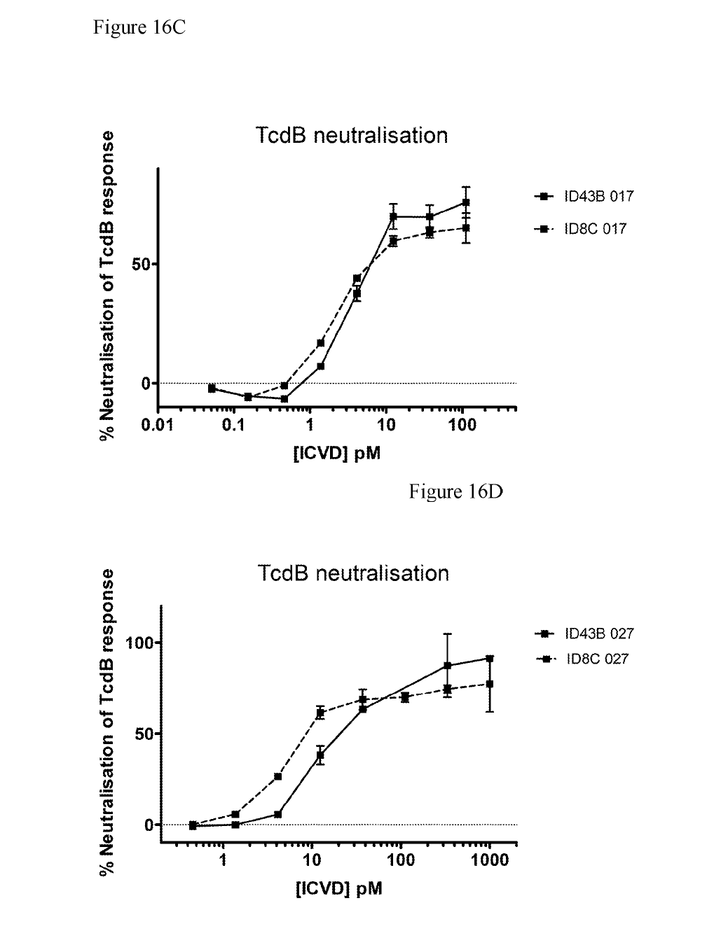

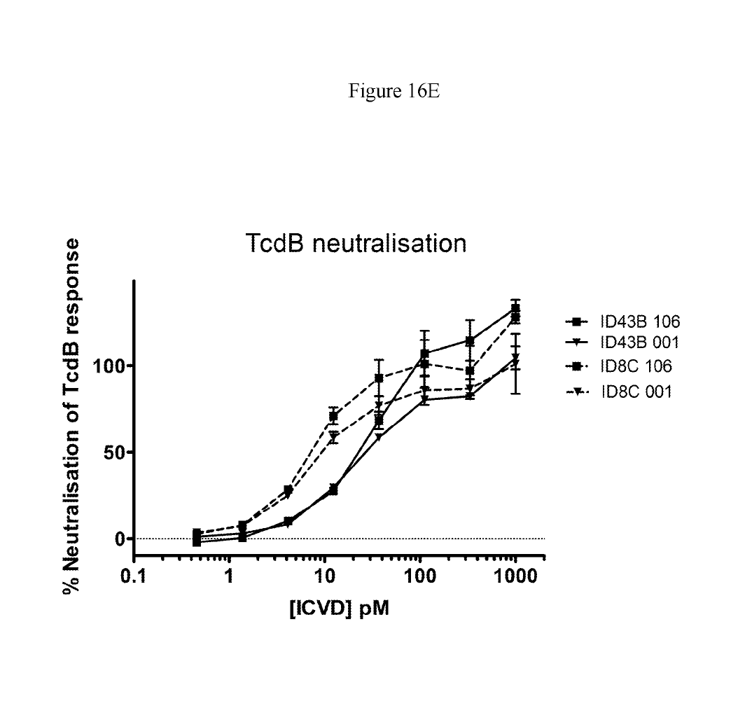

ID-8C is effectively a combination of anti-TcdA bihead ID-33A and anti-TcdB bihead ID43B. The neutralising potency of ID-8C was compared to that of: (a) constituent bihead ID-33A against TcdA from five ribotypes of C. difficile (FIG. 15, Graphs Ito III) and (b) constituent bihead ID43B against TcdB from six ribotypes of C. difficile (FIG. 16, Graphs Ito V). ID-8C demonstrated a similar or under certain circumstances even improved potency relative to its constituent biheads against both TcdA and TcdB from the various ribotypes tested.

### 8.3—Potency of ID-6C (ABB′A′)

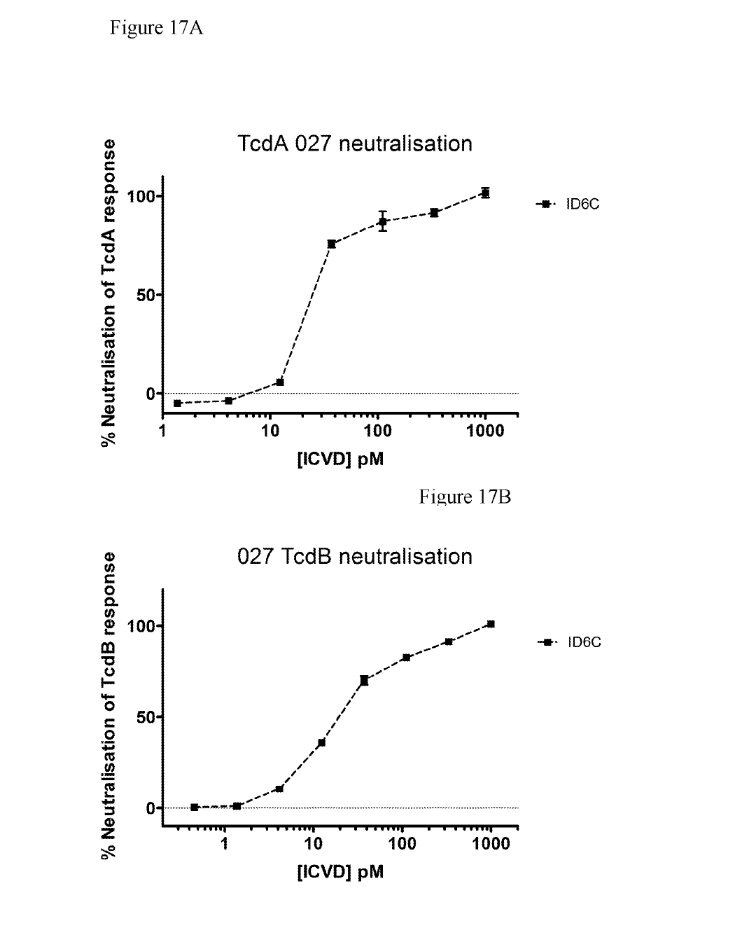

ID-6C was found to potently neutralise 027 TcdA (FIG. 17, Graph I) and 027 TcdB (FIG. 17, Graph II).

### 8.4—Potency of ID-7C and ID-11C (A′BB′A)

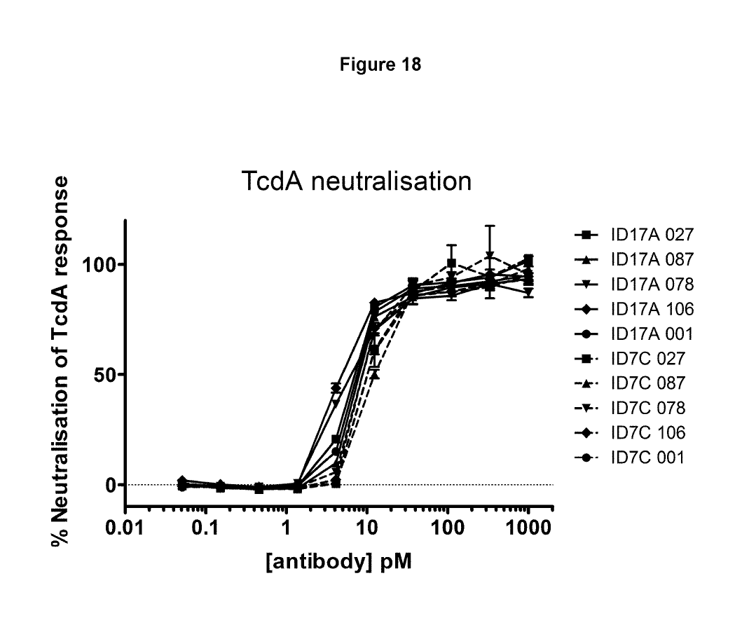

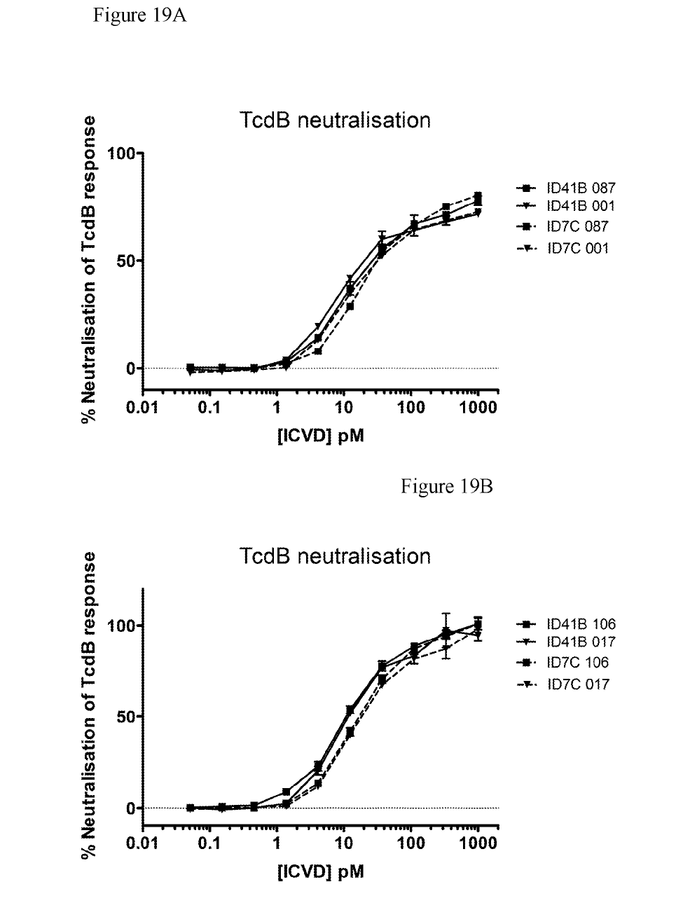

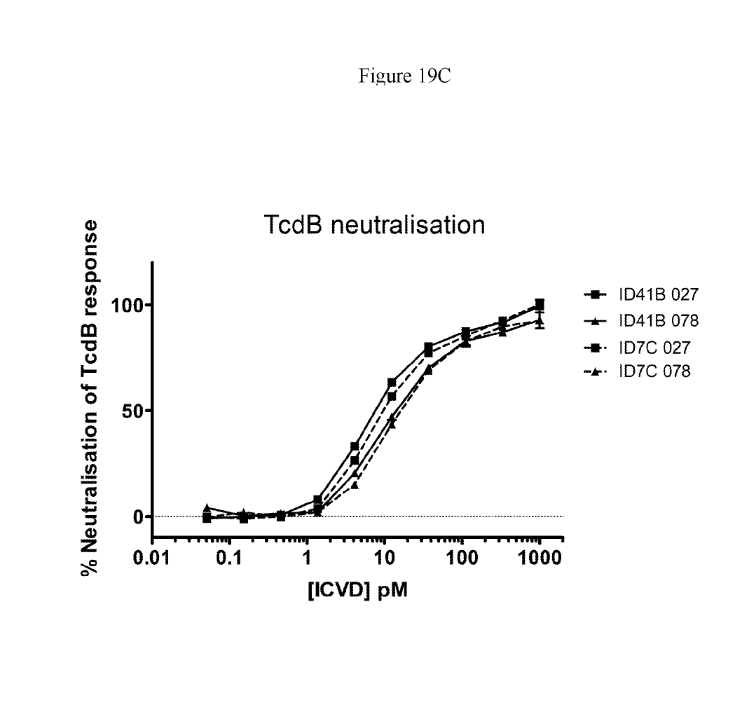

ID-7C is effectively a combination of anti-TcdA bihead ID-17A and anti-TcdB bihead ID41B. The neutralising potency of ID-8C was compared to that of: (a) constituent bihead ID-17A against TcdA from five ribotypes of C. difficile (FIG. 18) and (b) constituent bihead ID41B against TcdB from six ribotypes of C. difficile (FIG. 19, Graphs Ito III). ID-7C demonstrated a similar potency relative to its constituent biheads against both TcdA and TcdB from the various ribotypes tested.

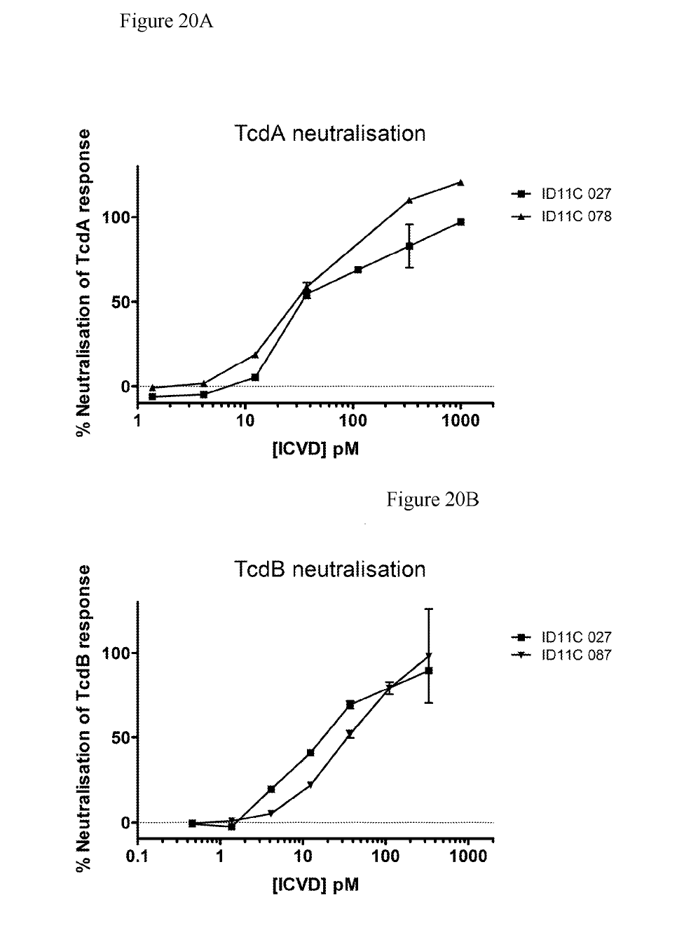

ID-11C (ID-7C with further CDR3 R to H modifications) also demonstrated potent neutralisation of 027 and 078 TcdA (FIG. 20, Graph I) and 027 and 087 TcdB (FIG. 20, Graph II).

### Example 9: Analysis of TcdA and TcdB Binding by ID-1C and ID-3C

An experiment was performed to investigate whether or not 1D-1C and ID-3C bind both TcdA and TcdB simultaneously.

Two ELISA plates were set up to investigate the binding of ID-1C and two to investigate the binding of ID-3C. Columns 1 to 3 of each plate were coated with 50 μl anti TcdA mAb PCG4.1. Columns 4 to 9 were coated with 027 TcdB or 087 TcdB. Columns 10 to 12 were left uncoated. The plates were incubated at 4° C. overnight, after which plates were washed, then all wells were blocked with 1% BSA in PBS for approximately 1.5 hours. Columns 6 to 12 were then incubated with 50 ul of (a) ID-1C, (b) ID-3C or (c) both ID-1C and ID-3C, for 1 hour. The Plates were then washed and incubated with various concentrations (0-20 ng/ml) of either 027 TcdA or 087 TcdA. After 2 hours of incubation, plates were washed, then incubated with rabbit anti-TcdA pAb or with rabbit polyclonal anti-ICVD for 1 hour. Plates were washed again and swine anti-rabbit-HRP was added to every well for 1 hour. After washing, plates were incubated with 100 μl TMB, allowed to develop, stopped with 50 μl of 0.5M H₂SO₄, then read at 450 nM in a plate reader.

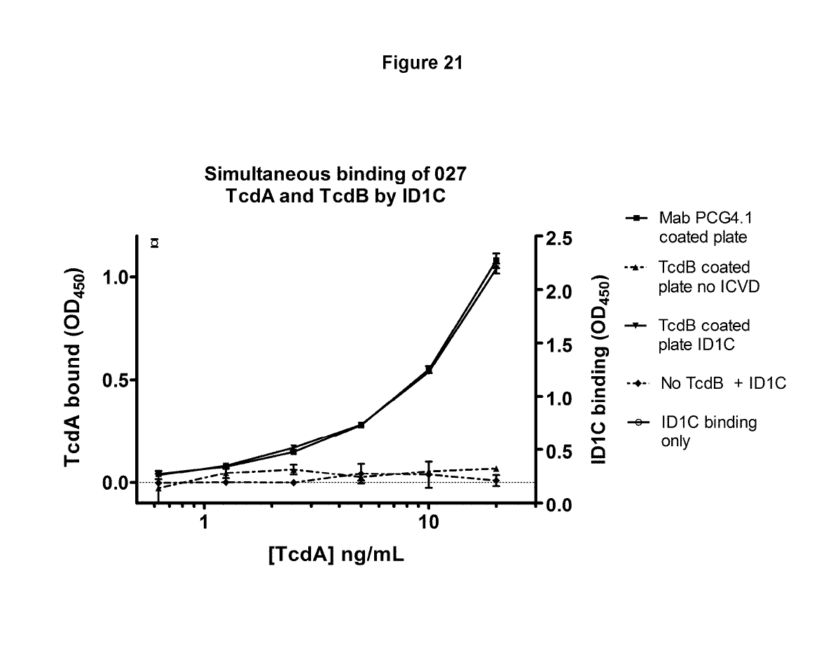

The results are shown in FIGS. 21 to 24, wherein ICVD binding in the form of quadraheads (open circle) is plotted on the right hand axis and amount of TcdA bound (closed symbols) is plotted on the left hand axis. As expected, the plates show a smooth dose response for the anti-TcdA mAb wells with increasing toxin concentration (square symbol). The other wells without any ICVD present in the form of quadrahead show no significant signal (dotted line, triangle symbol) and the wells without any TcdB show no significant signal (dotted line, diamond symbol). Clearly absence of ICVD in the form of quadraheads or anti-TcdA mAb, or absence of TcdB coating, completely negates TcdA binding) The signal from the rabbit polyclonal anti-ICVD wells demonstrated that the quadraheads had successfully bound to toxin (open circle). I

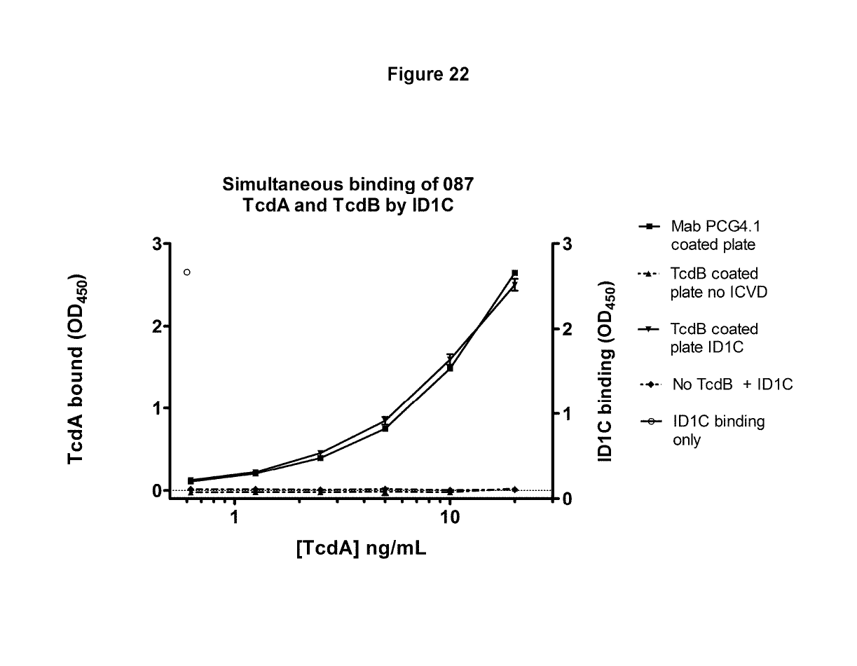

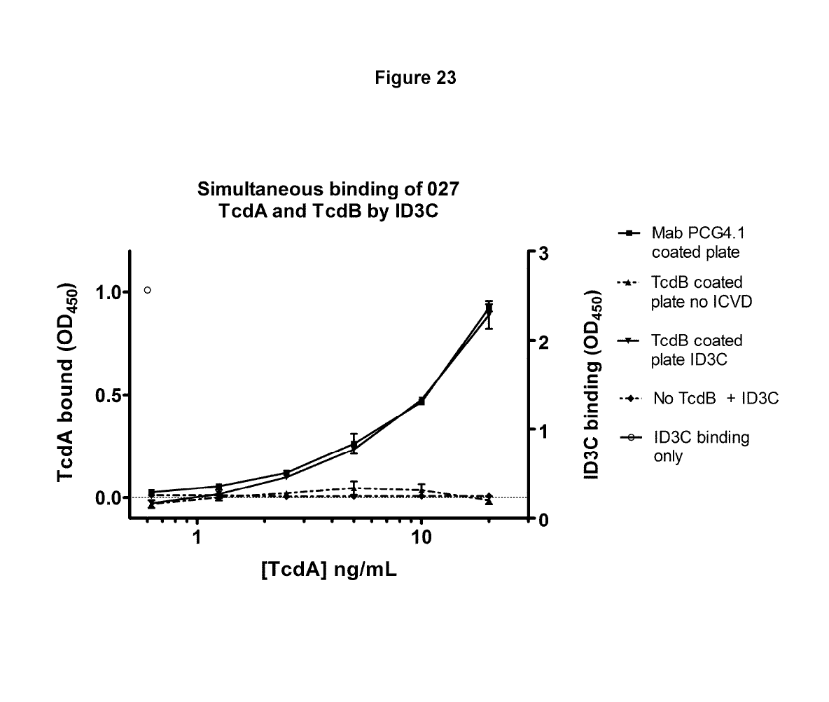

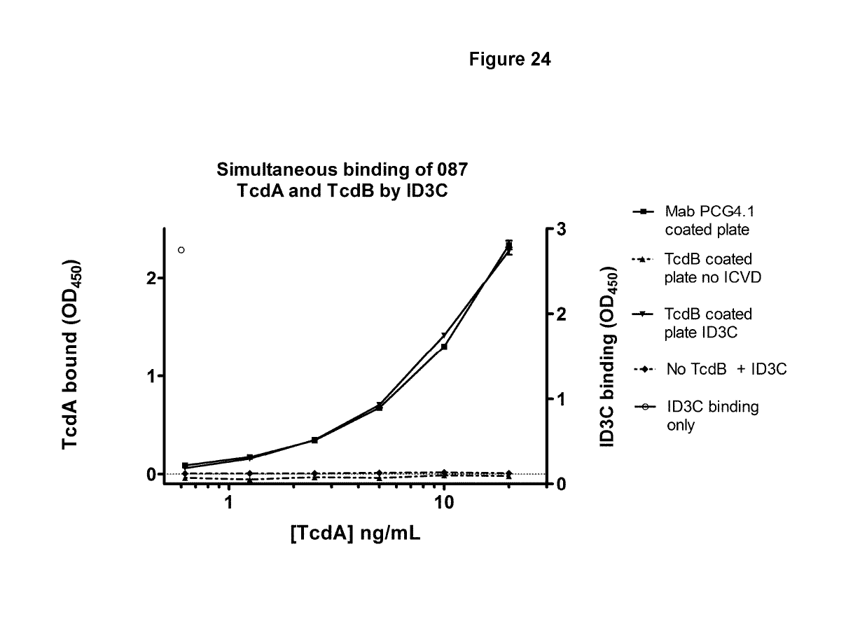

The 027 (FIGS. 21) and 087 (FIG. 22) ID-1C plates show smooth dose responses for ID-1C with increasing toxin concentration (unbroken line, triangle symbol) and the 027 (FIGS. 23) and 087 (FIG. 24) ID-3C plates show smooth dose responses for ID-3C with increasing toxin concentration (unbroken line, triangle symbol), demonstrating that both of these quadraheads successfully bind to the TcdB coatings and to the immobilised TcdA. It can be concluded therefore that quadraheads ID-1C and ID-3C simultaneously bind to TcdA and TcdB.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. All patents and patent applications mentioned throughout the specification of the present invention are herein incorporated in their entirety by reference. The invention embraces all combinations of preferred and more preferred groups and suitable and more suitable groups and embodiments of groups recited above.

1. A polypeptide comprising an immunoglobulin chain variable domain which binds to Clostridium difficile toxin A, wherein the immunoglobulin chain variable domain comprises three complementarity determining regions (CDR1-CDR3) and four framework regions (FR1-FR4), wherein CDR1 comprises a sequence sharing 40% or greater sequence identity with SEQ ID NO: 1, CDR2 comprises a sequence sharing 55% or greater sequence identity with SEQ ID NO: 2 and CDR3 comprises a sequence sharing 55% or greater sequence identity with SEQ ID NO: 3.

2. The polypeptide according to claim 1, wherein CDR1 comprises a sequence sharing 60% or greater sequence identity with SEQ ID NO: 1, CDR2 comprises a sequence sharing 60% or greater sequence identity with SEQ ID NO: 2 and CDR3 comprises a sequence sharing 60% or greater sequence identity with SEQ ID NO: 3.

3. The polypeptide according to claim 1, wherein any residues of CDR1, CDR2 or CDR3 differing from their corresponding residues in SEQ ID NO: 1, SEQ ID NO: 2 or SEQ ID NO: 3, respectively, are conservative substitutions with respect to their corresponding residues.

4. The polypeptide according to claim 1, wherein CDR1, CDR2 and/or CDR3 are devoid of K or R.

5. The polypeptide according to claim 1, wherein CDR1 comprises SEQ ID NO: 1, CDR2 comprises SEQ ID NO: 2 and CDR3 comprises SEQ ID NO: 3.

6. The polypeptide according to claim 5, wherein CDR1 consists of SEQ ID NO: 1, CDR2 consists of SEQ ID NO: 2 and CDR3 consists of SEQ ID NO: 3.

7. The polypeptide according to claim 1, wherein FR1, FR2, FR3 and FR4 each comprise a sequence sharing 40% or greater sequence identity with FR1, FR2, FR3 and FR4 of SEQ ID NO 14, respectively.

8. The polypeptide according to claim 1 which comprises or consists of SEQ ID NO: 14 or SEQ ID NO: 19.

9. The polypeptide according to claim 1, wherein the polypeptide is selected from the group consisting of an antibody and an antibody fragment such as a VHH, a VH, a VL, a V-NAR, a Fab fragment, a F(ab′)2 fragment or an scFv.

10. A construct comprising at least one polypeptide according to claim 1 and at least one further polypeptide, wherein the polypeptides are identical or different and wherein the polypeptides all bind to Clostridium difficile toxin A.

11. The construct according to claim 10, wherein the polypeptides are polypeptides according to claim 1.

12. A construct comprising at least one polypeptide according to claim 1 and at least one different polypeptide, wherein the different polypeptide binds to a target other than Clostridium difficile toxin A.

13. The construct according to claim 12, wherein the different polypeptide binds to Clostridium difficile toxin B.

14. The construct according to claim 12, wherein the construct comprises or consists of two polypeptides according to claim 1 and two polypeptides which bind to Clostridium difficile toxin B.

15. The construct according to claim 14 wherein the polypeptides are all connected by linkers.

16. The polypeptide according to claim 1, wherein the polypeptide is capable of neutralising Clostridium difficile ribotypes 087, 027, 078, 017, 106 and 001.

17. The polypeptide according to claim 1, which is substantially resistant to one or more proteases produced in the small or large intestine.

18. A pharmaceutical composition comprising the polypeptide according to claim 1 and one or more pharmaceutically acceptable diluents or carriers.

19. A method of treating Clostridium difficile infection comprising administering to a person in need thereof a therapeutically effective amount of the polypeptide according to claim 1.

20. A polypeptide which specifically binds to the epitope of Clostridium difficile toxin A bound by the polypeptide according to claim 1.


SEQUENCE LISTING (76 sequences)

SEQ ID NO: 1 - protein, 5 aa, Artificial Sequence
  CDR1 B4F10arm
VYAMG

SEQ ID NO: 2 - protein, 17 aa, Artificial Sequence
  CDR2 B4F10arm
TINRSGSDSYYADSVKG

SEQ ID NO: 3 - protein, 17 aa, Artificial Sequence
  CDR3 B4F10arm
SHSDCIGYGCHHVSQDY

SEQ ID NO: 4 - protein, 5 aa, Artificial Sequence
  CDR1 Q34A3arm
HKPAG

SEQ ID NO: 5 - protein, 16 aa, Artificial Sequence
  CDR2 Q34A3arm
SITTRASTHYADSVKG

SEQ ID NO: 6 - protein, 5 aa, Artificial Sequence
  CDR1 B14G12arm
NYNMA

SEQ ID NO: 7 - protein, 17 aa, Artificial Sequence
  CDR2 B14G12arm
VITWTGGSTYYAEDVKG

SEQ ID NO: 8 - protein, 17 aa, Artificial Sequence
  CDR3 B14G12arm
GLKGSGRSLSYPDGYDN

SEQ ID NO: 9 - protein, 5 aa, Artificial Sequence
  CDR1 B14G3arm
RNAAG

SEQ ID NO: 10 - protein, 16 aa, Artificial Sequence
  CDR2 B14G3arm
TLSSGGNTTYRDSVKG

SEQ ID NO: 11 - protein, 5 aa, Artificial Sequence
  CDR1 Q30A5
TYDMS

SEQ ID NO: 12 - protein, 17 aa, Artificial Sequence
  CDR2 Q30A5arm
AINPGGTSTYYADSVKG

SEQ ID NO: 13 - protein, 15 aa, Artificial Sequence
  CDR3 Q30A5arm
SYASGIDYYPPPRDS

SEQ ID NO: 14 - protein, 126 aa, Artificial Sequence
  B4F10arm
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASHSDCIGYGCHHVSQDYWGQGTQVTVSS

SEQ ID NO: 15 - protein, 111 aa, Artificial Sequence
  Q34A3arm
DVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 16 - protein, 126 aa, Artificial Sequence
  B14G12arm
DVQLQESGGGLVQAGGSLRLSCAASGLSISNYNMAWFRQAPGKERELVAVITWTGGSTYYAEDVKGRLTISRDNAKNTVFLQMNSLKPEDTAIYFCAAGLKGSGRSLSYPDGYDNWGQGTQVTVSS

SEQ ID NO: 17 - protein, 111 aa, Artificial Sequence
  B14G3arm
DVQLQESGGGLVQAGGSLRLSCAASGHIFSRNAAGWFRQAPGKQREAVATLSSGGNTTYRDSVKGRFTISRDDTTNTVHLQMNSLKPEDTAVYYCNAMWYWGQGTQVTVSS

SEQ ID NO: 18 - protein, 124 aa, Artificial Sequence
  Q30A5arm
DVQLQESGGGLVQPGGSLKLSCAASGFTFGTYDMSWVRQAPGKRPEWVSAINPGGTSTYYADSVKGRFTVSRDNAKDTLYLQMNSLKPEDTAVYYCTPSYASGIDYYPPPRDSWGQGTQVTVSS

SEQ ID NO: 19 - protein, 126 aa, Artificial Sequence
  B4F10
QVQLQETGGGLVQAGGSLRLSCAASGRTSDVYAMGWFRQVPGKEREFVATINRSGSDSYYADSVKGRFTISRDNAKNTVYLQMNSLKPEETAVYYCAASRSDCIGYGCRRVSQDYWGQGTQVTVSS

SEQ ID NO: 20 - protein, 126 aa, Artificial Sequence
  B1A7
QVQLQESGGGLVQAGGSLRLSCEASGRTSSIYTMGWFRQVPGKEREFVAGTSRSGTGSYYADSVKGRFTISRDNAKNTVYLQMNSLKPEETAVYYCAASRSDCLGVGCRRVPYEYWGQGTQVTVSS

SEQ ID NO: 21 - protein, 126 aa, Artificial Sequence
  B2H11
QVQLQESGGGLVQAGGSLRLSCAASGRTDDVYAMGWFRQVPGKEREFVATINRSGSDSFYADSVKGRFTISRDNAKNTVYLQMNSLKPEETAVYYCAASRSDCIGYGCRRVPQEYWGQGTQVTVSS

SEQ ID NO: 22 - protein, 126 aa, Artificial Sequence
  B3D5
QVQLQESGGGLVQAGGSLRLSCAASGQTFDVYGMGWFRQVPGKEREFVAGTSRSGTGSYYADSVKGRFTISRDNAKNTVYLQMNSLKPEETAVYYCAASRSDCLGVGCRRVPYEYWGQGTQVTVSS

SEQ ID NO: 23 - protein, 126 aa, Artificial Sequence
  B3H7
QVQLQESGGGSVQAGGSLRLSCAASGHTSDVYTMGWFRQVPGKEREFVATINRSGSDSYYADSVKGRFTISRDNAKNTVYLQMNSLKPEETAVYYCAASRSDCLGIGCRRVPHDYWGQGTQVTVSS

SEQ ID NO: 24 - protein, 126 aa, Artificial Sequence; identical to SEQ ID NO: 21
  B3H11

SEQ ID NO: 25 - protein, 126 aa, Artificial Sequence
  B4F5
QVQLQESGGGLVQAGGSLTLSCVASGRTSDVYAMGWFRQVPGKEREFVATINRSGSDAYYADSVKGRFTISRDNAKNTVYLQMNSLKPEETAVYYCAASRSDCIGYGCRRVSHNYWGQGTQVTVSS

SEQ ID NO: 26 - protein, 126 aa, Artificial Sequence
  B4F7
QVQLQESGGGLVQAGGSLRLSCAASGHTSDVYTMGWFRQVPGKEREFVATINRSGTDSYYADSVKGRFTISRDNAKNTVYLQMNSLKPEETAVYYCAASRSDCLGIGCRRVPHDYWGQGTQVTVSS

SEQ ID NO: 27 - protein, 126 aa, Artificial Sequence
  B4G7
QVQLQETGGGLVHAGGSLRLSCAASGHTSDVYTMGWFRQVPGKEREFVATINRSGSDSYYADSVKGRFTISRDNAKNTVYLQMNSLKPEETAVYYCAASRSDCLGIGCRRVPHDYWGQGTQVTVSS

SEQ ID NO: 28 - protein, 126 aa, Artificial Sequence
  B4H7
QVQLQESGGGLVQAGGSLRLSCAASGRTDDVYAMGWFRQVPGKEREFVATISRGGSDSYYADSVKGRFTISRDNAKNTVYLQMNSLKPEETAVYYCAASRSDCLGNGCRRVPQDYWGQGTQVTVSS

SEQ ID NO: 29 - protein, 111 aa, Artificial Sequence
  Q34A3
EVQLVESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 30 - protein, 126 aa, Artificial Sequence
  B14G12
QVQLQESGGGLVQAGGSLRLSCAASGLSISNYNMAWFRQAPGKERELVAVITWTGGSTYYAEDVKGRLTISRDNAKNTVFLQMNSLKPEDTAIYFCAAGLKGSGRSLSYPDGYDNWGQGTQVTVSS

SEQ ID NO: 31 - protein, 111 aa, Artificial Sequence
  B14G3
QVQLQESGGGLVQAGGSLRLSCAASGHIFSRNAAGWFRQAPGKQREAVATLSSGGNTTYRDSVKGRFTISRDDTTNTVHLQMNSLKPEDTAVYYCNAMWYWGQGTQVTVSS

SEQ ID NO: 32 - protein, 124 aa, Artificial Sequence
  Q30A5
RGAAGESGGGLVQPGGSLKLSCAASGFTFGTYDMSWVRQAPGKRPEWVSAINPGGTSTYYADSVKGRFTVSRDNAKDTLYLQMNSLKPEDTAVYYCTPSYASGIDYYPPPRDSWGQGTQVTVSS

SEQ ID NO: 33 - protein, 111 aa, Artificial Sequence
  ID1A
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 34 - protein, 111 aa, Artificial Sequence
  ID2A
DVQLQESGGGLVQAGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 35 - protein, 242 aa, Artificial Sequence
  ID3A
DVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSSGGGGSGGGGSGGGGSGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 36 - protein, 111 aa, Artificial Sequence
  ID10A
DVQLQESGGGLVQPGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 37 - protein, 257 aa, Artificial Sequence
  ID11A
DVQLQESGGGLVQAGGSLRLSCAASGLSISNYNMAWFRQAPGKERELVAVITWTGGSTYYAEDVKGRLTISRDNAKNTVFLQMNSLKPEDTAIYFCAAGLKGSGRSLSYPDGYDNWGQGTQVTVSSGGGGSGGGGSGGGGSGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 38 - protein, 257 aa, Artificial Sequence
  ID12A
DVQLQESGGGLVQAGGSLRLSCAASGRTSDVYAMGWFRQVPGKEREFVATINRSGSDSYYADSVKGRFTISRDNAKNTVYLQMNSLKPEETAVYYCAASRSDCIGYGCRRVSQDYWGQGTQVTVSSGGGGSGGGGSGGGGSGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 39 - protein, 242 aa, Artificial Sequence
  ID13A
DVQLQESGGGLVQAGGSLRLSCAASGHIFSRNAAGWFRQAPGKQREAVATLSSGGNTTYRDSVKGRFTISRDDTTNTVHLQMNSLKPEDTAVYYCNAMWYWGQGTQVTVSSGGGGSGGGGSGGGGSGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 40 - protein, 255 aa, Artificial Sequence
  ID15A
DVQLQESGGGLVQPGGSLKLSCAASGFTFGTYDMSWVRQAPGKRPEWVSAINPGGTSTYYADSVKGRFTVSRDNAKDTLYLQMNSLKPEDTAVYYCTPSYASGIDYYPPPRDSWGQGTQVTVSSGGGGSGGGGSGGGGSGGGGSDVQLQESGGGLVQPGGSLRLSCVISGMDFSHKPAGWFRQAPGKEREFVASITTRASTHYADSVKGRFTISRDNAKNTVYLEMNSLKPEDTAVYSCNSEYYWGQGTQVTVSS

SEQ ID NO: 41 - protein, 257 aa, Artificial Sequence
  ID17A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCRRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 42 - protein, 257 aa, Artificial Sequence
  ID29A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCHRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 43 - protein, 257 aa, Artificial Sequence
  ID30A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCRHVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 44 - protein, 257 aa, Artificial Sequence
  ID31A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCHHVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 45 - protein, 257 aa, Artificial Sequence
  ID32A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASHSDCIGYGCRRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 46 - protein, 257 aa, Artificial Sequence
  ID33A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASHSDCIGYGCHHVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 47 - protein, 257 aa, Artificial Sequence
  ID34A
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGHGCHRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 48 - protein, 257 aa, Artificial Sequence
  ID35A
DVQLVESGGGLVQAGGSLRLSCAASGATSDVYAMGWFRQVPGKEREFVATINRSGSDSYYADSVKGRFTISRDNAKNTVYLQMNSLKPEETAVYYCAASHSDCIGYGCHHVSQDYWGQGTQVTVSSGGGGSGGGGSGGGGSGGGGSDVQLVESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 49 - DNA, 28 bp, Artificial Sequence
  3′ primer
tcttaactagtgaggagacggtgacctg

SEQ ID NO: 50 - DNA, 17 bp, Artificial Sequence
  M13.rev
caggaaacagctatgac

SEQ ID NO: 51 - DNA, 16 bp, Artificial Sequence
  M13.fw
gtaaaacgacggccag

SEQ ID NO: 52 - DNA, 339 bp, Artificial Sequence
  Polynucleotide sequence encoding ID1A
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 53 - DNA, 339 bp, Artificial Sequence
  Polynucleotide sequence encoding ID2A
gatgtgcagctgcaggagtctgggggaggcttggtgcaggctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 54 - DNA, 339 bp, Artificial Sequence
  Polynucleotide sequence encoding ID10A
gatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattattgtaactccgaatactactggggccaggggacccaggtcaccgtctcctcataatga

SEQ ID NO: 55 - DNA, 777 bp, Artificial Sequence
  Polynucleotide sequence encoding ID11A
gatgtgcagctgcaggagtccgggggaggattggtgcaggctgggggctctctgagactctcctgtgcagcctctggactcagcatcagtaactataacatggcctggttccgccaggctccagggaaggagcgtgagttggtagcagtgattacctggactggcggtagcacatactatgcagaggacgtgaagggccgactcaccatctccagagacaacgccaagaacacggtgtttctccagatgaacagcctgaaacctgaggacacggccatttacttctgtgcagcaggattaaagggtagtggccggtccctaagttatccggatggttatgacaactggggccaggggacccaggtcaccgtctcctcaggtggaggcggttcaggcggaggtggctctggcggtggcggaagtggtggaggcggttcagatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 56 - DNA, 777 bp, Artificial Sequence
  Polynucleotide sequence encoding ID12A
gatgtgcagctgcaggagtctgggggaggattggtgcaggctgggggctctctgagactctcctgtgcagcctctggacgcaccagcgatgtctatgccatgggctggttccgccaggttccaggaaaggagcgcgaatttgtagcgactattaaccggagtggtagtgactcatactatgcggactccgtgaagggccggttcaccatctccagagacaacgccaagaacacggtgtatctgcaaatgaacagcctgaaacctgaggaaacggccgtttattactgtgcggcctctaggagcgactgtataggttatgggtgtcgtcgcgtctcgcaagactactggggccaggggacccaggtcaccgtctcctcaggtggaggcggttcaggcggaggtggctctggcggtggcggaagtggtggaggcggttcagatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 57 - DNA, 732 bp, Artificial Sequence
  Polynucleotide sequence encoding ID13A
gatgtgcagctgcaggagtccgggggaggcttggtgcaggctggggggtctctgagactctcctgtgcagcctcgggacacatcttcagtcgcaatgccgcgggctggttccgccaggctccagggaagcagcgcgaggcggtcgcgacactaagtagtggtggaaatacaacctatcgagactccgtgaagggccgattcaccatctccagagacgacaccacgaacacggtgcatctgcaaatgaacagcctgaaacctgaggacacggccgtctattactgtaatgctatgtggtactggggccaggggacccaggtcaccgtctcctcaggtggaggcggttcaggcggaggtggctctggcggtggcggaagtggtggaggcggttcagatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 58 - DNA, 771 bp, Artificial Sequence
  Polynucleotide sequence encoding ID15A
gatgtgcagctgcaggagtctgggggaggcttggtgcagcctgggggttctctgaaactctcctgtgcagcctccggattcaccttcgggacgtatgacatgagctgggtccgccaggctccaggaaagaggcccgagtgggtctcagcgattaatccgggtggtactagcacatactatgcagactccgtgaagggccgattcaccgtctccagagacaacgccaaggacacgctgtatctgcaaatgaacagcctgaaacctgaggacacggccgtgtattactgtactccgtcatacgctagtggtattgactactacccccccccgcgagattcctggggccaggggacccaggtcaccgtctcctcaggtggaggcggttcaggcggaggtggctctggcggtggcggaagtggtggaggcggttcagatgtgcagctgcaggagtctgggggaggcttggtgcagcctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattcttgtaactccgaatactactggggccaggggacccaggtcaccgtctcctcataatga

SEQ ID NO: 59 - DNA, 777 bp, Artificial Sequence
  Polynucleotide sequence encoding ID17A
gatgtgcagctgcaggagtctgggggaggattggtgcaggctgggggctctctgagactctcctgtgcagcctctggagctaccagcgatgtctatgccatgggctggttccgccaggttccaggaaaggagcgcgaatttgtagcgactattaaccggagtggtagtgactcatactatgcggactccgtgaagggccggttcaccatctccagagacaacgccaagaacacggtgtatctgcaaatgaacagcctgaaacctgaggaaacggccgtttattactgtgcggcctctaggagcgactgtataggttatgggtgtcgtcgcgtctcgcaagactactggggccaggggacccaggtcaccgtctcctcaggtggaggcggttcaggcggaggtggctctggcggtggcggaagtggtggaggcggttcagatgtgcagctgcaggagtctgggggaggcttggtgcaggctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattattgtaactccgaatactactggggccaggggacccaggtcaccgtctcctcataatga

SEQ ID NO: 60 - DNA, 777 bp, Artificial Sequence
  Polynucleotide sequence encoding ID33A
gatgtgcagctgcaggagtctgggggaggattggtgcaggctgggggctctctgagactctcctgtgcagcctctggagctaccagcgatgtctatgccatgggctggttccgccaggttccaggaaaggagcgcgaatttgtagcgactattaaccggagtggtagtgactcatactatgcggactccgtgaagggccggttcaccatctccagagacaacgccaagaacacggtgtatctgcaaatgaacagcctgaaacctgaggaaacggccgtttattactgtgcggcctctcatagcgactgtataggttatgggtgtcatcacgtctcgcaagactactggggccaggggacccaggtcaccgtctcctcaggtggaggcggttcaggcggaggtggctctggcggtggcggaagtggtggaggcggttcagatgtgcagctgcaggagtctgggggaggcttggtgcaggctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattattgtaactccgaatactactggggccaggggacccaggtcaccgtctcctcataatga

SEQ ID NO: 61 - DNA, 339 bp, Artificial Sequence
  Polynucleotide sequence encoding Q34A3arm (in ID33A)
gatgtgcagctgcaggagtctgggggaggcttggtgcaggctggggggtctctgagactctcctgtgtaatctctggaatggacttcagtcacaaacccgcgggctggttccgccaggctccaggaaaagagcgcgagttcgtcgcttcgattacgactcgtgctagcacgcactatgcagactccgtgaagggccgattcaccatctccagagacaacgccaagaacacggtatatctagaaatgaacagcctgaaacctgaggacacggccgtctattattgtaactccgaatactactggggccaggggacccaggtcaccgtctcctcataatga

SEQ ID NO: 62 - DNA, 384 bp, Artificial Sequence
  Polynucleotide sequence encoding B4F10arm (in ID33A)
gatgtgcagctgcaggagtctgggggaggattggtgcaggctgggggctctctgagactctcctgtgcagcctctggagctaccagcgatgtctatgccatgggctggttccgccaggttccaggaaaggagcgcgaatttgtagcgactattaaccggagtggtagtgactcatactatgcggactccgtgaagggccggttcaccatctccagagacaacgccaagaacacggtgtatctgcaaatgaacagcctgaaacctgaggaaacggccgtttattactgtgcggcctctcatagcgactgtataggttatgggtgtcatcacgtctcgcaagactactggggccaggggacccaggtcaccgtctcctcataatga

SEQ ID NO: 63 - protein, 543 aa, Artificial Sequence
  ID1C
DVQLQESGGGLVQAGGSLRLSCAASGATSDVYAIGWFRQVPGKEREFVATINRSGSDSYYADSVKGRFTISRDNAKNTVYLQMNSLKPEETAVYYCAASRSDCIGYGCRRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSSGGGGSGGGGSGGGGSGGGGSDVQLQESGGGLVQAGDSLRLSCAASGATLSSYTIGWFRQAPEKEREFVAGSSRDGRTNYYANSVKGRFTISRDNAKNTVYLQMNSLKPEDTAVYYCAAHTTSGVPVRERSYAYWGQGTQVTVSSGGGGSGGGGSGGGGSGGGGSDVQLQESGGGLVQAGGSLRLSCAASGATFSSYYIGWFRQAPGKEREFVAAINGSGGNRISADSVKGRFTISRDNAKNTVYLQLNSLKPEDTAVYYCAASLTYYGRSARYDYWGQGTQVTVSS

SEQ ID NO: 64 - protein, 543 aa, Artificial Sequence
  ID3C
DVQLQESGGGLVQAGDSLRLSCAASGATLSSYTIGWFRQAPEKEREFVAGSSRDGRTNYYANSVKGRFTISRDNAKNTVYLQMNSLKPEDTAVYYCAAHTTSGVPVRERSYAYWGQGTQVTVSSGGGGSGGGGSGGGGSGGGGSDVQLQESGGGLVQAGGSLRLSCAASGATSDVYAIGWFRQVPGKEREFVATINRSGSDSYYADSVKGRFTISRDNAKNTVYLQMNSLKPEETAVYYCAASRSDCIGYGCRRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSSGGGGSGGGGSGGGGSGGGGSDVQLQESGGGLVQAGGSLRLSCAASGATFSSYYIGWFRQAPGKEREFVAAINGSGGNRISADSVKGRFTISRDNAKNTVYLQLNSLKPEDTAVYYCAASLTYYGRSARYDYWGQGTQVTVSS

SEQ ID NO: 65 - protein, 543 aa, Artificial Sequence
  ID5C
DVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSARYDYWGQGTQVTVSSGGGGSGGGGSGGGGSGGGGSDVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCRRVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 66 - protein, 543 aa, Artificial Sequence
  ID6C
DVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCRRVSQDYWGQGTQVTVSSGGGGSGGGGSGGGGSGGGGSDVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSARYDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 67 - protein, 543 aa, Artificial Sequence
  ID7C
DVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSSGGGGSGGGGSGGGGSGGGGSDVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSARYDYWGQGTQVTVSSGGGGSGGGGSGGGGSGGGGSDVQLQESGGGLVQAGGSLRLSCAASGATSDVYAMGWFRQVPGKEREFVATINRSGSDSYYADSVKGRFTISRDNAKNTVYLQMNSLKPEETAVYYCAASRSDCIGYGCRRVSQDYWGQGTQVTVSS

SEQ ID NO: 68 - protein, 543 aa, Artificial Sequence
  ID8C
DVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSAHYDYWGQGTQVTVSSGGGGSGGGGSGGGGSGGGGSDVQLQESGGGLVQAGGSLRLSCAASGATSDVYAMGWFRQVPGKEREFVATINRSGSDSYYADSVKGRFTISRDNAKNTVYLQMNSLKPEETAVYYCAASHSDCIGYGCHHVSQDYWGQGTQVTVSSGGGGSGGGGSGGGGSGGGGSDVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSS

SEQ ID NO: 69 - protein, 543 aa, Artificial Sequence
  ID11C
DVQLQESGGGLVQAGGSLRLSCVISGMDFSHKPAGWFRQAPGKEREFVASITTRASTHYADSVKGRFTISRDNAKNTVYLEMNSLKPEDTAVYYCNSEYYWGQGTQVTVSSGGGGSGGGGSGGGGSGGGGSDVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSAHYDYWGQGTQVTVSSGGGGSGGGGSGGGGSGGGGSDVQLQESGGGLVQAGGSLRLSCAASGATSDVYAMGWFRQVPGKEREFVATINRSGSDSYYADSVKGRFTISRDNAKNTVYLQMNSLKPEETAVYYCAASHSDCIGYGCHHVSQDYWGQGTQVTVSS

SEQ ID NO: 70 - protein, 124 aa, Artificial Sequence
  Q31B1
EVQLVESGGGLVQAGDSLRLSCAASGRTLSSYTMGWFRQAPEKEREFVAGSSRDGRTNYYANSVKGRFTISRDNAKNTVYLQMNSLKPEDTAVYYCAAHTTSGVPVRERSYAYWGQGTQVTVSS

SEQ ID NO: 71 - protein, 122 aa, Artificial Sequence
  B10F1
QVQLQESGGGLVQAGGSLRLSCAASGRTFSSYYMGWFRQAPGKEREFVAAINGSGGNRISADSVKGRFTISRDNAKNTVYLQLNSLKPEDTAVYYCAASLTYYGRSARYDYWGQGTQVTVSS

SEQ ID NO: 72 - protein, 266 aa, Artificial Sequence
  ID43B
DVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSAHYDYWGQGTQVTVSS

SEQ ID NO: 73 - protein, 266 aa, Artificial Sequence
  ID41B
DVQLQESGGGLVQAGDSLRLSCAASGATLSSYTMGWFRQAPEKEREFVAGSSRDGRTNYYANSVKGRFTISRDNAKNTVYLQMNSLKPEDTAVYYCAAHTTSGVPVHERSYAYWGQGTQVTVSSGGGGSGGGGSGGGGSGGGGSDVQLQESGGGLVQAGGSLRLSCAASGATFSSYYMGWFRQAPGKEREFVAAINGSGGNRISADSVKGRFTISRDNAKNTVYLQLNSLKPEDTAVYYCAASLTYYGHSARYDYWGQGTQVTVSS

SEQ ID NO: 74 - protein, 2710 aa, Artificial Sequence
  Polypeptide sequence of TcdA from C. difficile ribotype 087
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGLQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 75 - protein, 2710 aa, Artificial Sequence
  Polypeptide sequence of TcdA from C. difficile ribotype 078
MSLISKEELIKLAYSIRPRENEYKTILTNLDEYNKLTTNNNENKYLQLKKLNESIDVFMNKYKNSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFVNYYKSQINKPTVPTIDDIIKSHLVSEYNRDETLLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNEVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKDSITIGANQYEVRINSEGRKELLAHSGKWINKEEAIMSDLSSKEYIFFDSIDNKLKAKSKNIPGLASISEDIKTLLLDTSVSPDTKFILNNLKLNIESSVGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDLLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKEYGPLKTEDDKILVPIDDLVISEIDFNNNAIKLGTCNILAMEGGSGHTVTGNIDHFFSSPSISSHIPSLSIYSAIGIKTENLDFSKKIMMLPNAPSRVFWWETGAVPGLRSLENNGTKLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNGSTLEFNSKDFIAEDINVFMKDDINTITGKYYVDNNTDKSIDFSISLVSKNQAKVNGLYLNESVYSSYLDFVKNADGHHNTSNFMNLFLDNISFWKLFGFENINFVVDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLVYIMSNFKSFNSENELDRDHLGFKIIDNKTYYYDEASKLVKGLININNSLFYFDPIESNLVTGWQTINGKKYYFDINTGAALTSYKIINGKHFYFNNNGVMQLGVFKGPDRFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSGSNGFEYFAPANTYNNNIEGQAIVYQSKFLTLNGKKYYFDNNSKAVTGWQTIDSKKYYFNTNTAEAATGWQTIDGKKYYFNTNTAEVATGWQTIDGKKYYFNTNTSIASTGYTIINGKHFYFNTDGIMQIGVFKGPDGFEYFAPANTDANNIEGQAILYQNKFLTLNGKKYYFASDSKAVTGWRIINNKKYYFNPNNAIAATHLYTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAVAVTGWQTIDGKKYYFNTNTAEAATGWQTIDGKKYYFNTNTSIASTGYTIINGKHFYFNTDGIMQIGVFKGPDGFEYFAPANTDANNIEGQAILYQNKFLTLNGKKYYFASDSKAVTGLRTIDGKKYYFNTNTAEAATGWQTIDGKKYYFNTNTSIASTGYTIINDKHFYFNTDGIMQIGVFKGPDGFEYFAPANTDANNIEGQAIRYQNRFLYLHDNIYYFGNNSKAATGWVTIDGRRYYFEPNTAIGANGYKIIDNKNFYFRNGLPQIGVFKGPNGFEYFAPANTDANNIDGQAIRYQNRFLHLLGNIYYFGNNSKAVTGWQTINGNMYYFMPDTAMAAAGGLFEIDGVIYFFGVDGVKAPGIYG

SEQ ID NO: 76 - protein, 2710 aa, Artificial Sequence
  Polypeptide sequence of TcdA from C. difficile ribotype 027
MSLISKEELIKLAYSIRPRENEYKTILTNLDEYNKLTTNNNENKYLQLKKLNESIDVFMNKYKNSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETLLESYRTNSLRKINSNHGIDIRANSLFTEQELLNIYSQELLNRGNLAAASDIVRLLALKNFGGVYLDVDMLPGIHSDLFKTIPRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKDSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKEYGPLKTEDDKILVPIDDLVISEIDFNNNSIKLGTCNILAMEGGSGHTVTGNIDHFFSSPYISSHIPSLSVYSAIGIKTENLDFSKKIMMLPNAPSRVFWWETGAVPGLRSLENNGTKLLDSIRDLYPGKFYWRFYAFFDYAITTLKPVYEDTNTKIKLDKDTRNFIMPTITTDEIRNKLSYSFDGAGGTYSLLLSSYPISMNINLSKDDLWIFNIDNEVREISIENGTIKKGNLIEDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNGSTLEFNSKDFIAEDINVFMKDDINTITGKYYVDNNTDKSIDFSISLVSKNQVKVNGLYLNESVYSSYLDFVKNSDGHHNTSNFMNLFLNNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIESNLVTGWQTINGKKYYFDINTGAASTSYKIINGKHFYFNNNGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSGSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTSIASTGYTIINGKYFYFNTDGIMQIGVFKVPNGFEYFAPANTHNNNIEGQAILYQNKFLTLNGKKYYFGSDSKAITGWQTIDGKKYYFNPNNAIAATHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDSKKYYFNLNTAVAVTGWQTIDGEKYYFNLNTAEAATGWQTIDGKRYYFNTNTYIASTGYTIINGKHFYFNTDGIMQIGVFKGPDGFEYFAPANTHNNNIEGQAILYQNKFLTLNGKKYYFGSDSKAVTGLRTIDGKKYYFNTNTAVAVTGWQTINGKKYYFNTNTYIASTGYTIISGKHFYFNTDGIMQIGVFKGPDGFEYFAPANTDANNIEGQAIRYQNRFLYLHDNIYYFGNDSKAATGWATIDGNRYYFEPNTAMGANGYKTIDNKNFYFRNGLPQIGVFKGPNGFEYFAPANTDANNIDGQAIRYQNRFLHLLGKIYYFGNNSKAVTGWQTINSKVYYFMPDTAMAAAGGLFEIDGVIYFFGVDGVKAPGIYG